(19) United States
(12) Patent Application Publication
(10) Pub. No.: US 2015/0179956 A1
(43) Pub. Date: Jun. 25, 2015

(54) MATERIAL FOR ORGANIC ELECTROLUMINESCENCE DEVICE AND ORGANIC ELECTROLUMINESCENCE DEVICE USING THE SAME

(71) Applicant: SAMSUNG DISPLAY CO., LTD., Yongin-City (KR)

(72) Inventors: Yasuo MIYATA, Yokohama (JP); Hiroaki ITOI, Yokohama (JP)

(21) Appl. No.: 14/577,015

(22) Filed: Dec. 19, 2014

(30) Foreign Application Priority Data
Dec. 20, 2013 (JP) 2013-264433

(51) Int. Cl. H01L 51/00; C07F 7/10; C07D 487/06

(52) U.S. Cl. CPC H01L 51/0072; C07D 487/06; H01L 51/0061; H01L 51/0073; C07F 7/10; H01L 51/0094; H01L 51/5056

(57) ABSTRACT

A material for an organic electroluminescence (EL) device and an organic EL device, the material being represented by the following Formula 1:

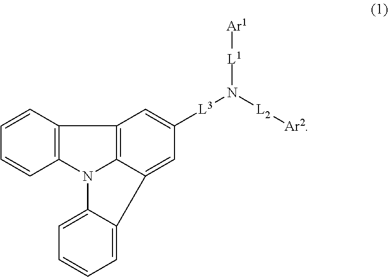

### CROSS-REFERENCE TO RELATED APPLICATION

Japanese Patent Application No. 2013-264433, filed on Dec. 20, 2013, in the Japanese Patent Office, and entitled: “Material for Organic Electroluminescence Device and Organic Electroluminescence Device Using the Same,” is incorporated by reference herein in its entirety.

### BACKGROUND

1. Field

Embodiments relate to a material for an organic electroluminescence device and an organic electroluminescence device using the same.

2. Description of the Related Art

In recent years, organic electroluminescence (EL) displays that are one type of image displays have been actively developed. Unlike a liquid crystal display and the like, the organic EL display is a self-luminescent display that recombines holes and electrons injected from a anode and a cathode in an emission layer to thus emit light from a light-emitting material including an organic compound of the emission layer, thereby performing display.

An example of an organic electroluminescence device (hereinafter referred to as an organic EL device) includes an organic EL device that includes a anode, a hole transport layer disposed on the anode, an emission layer on the hole transport layer, an electron transport layer on the emission layer, and a cathode on the electron transport layer. Holes injected from the anode may be injected into the emission layer via the hole transport layer. Meanwhile, electrons may be injected from the cathode, and then injected into the emission layer via the electron transport layer. The holes and the electrons injected into the emission layer may be recombined to generate excitons within the emission layer. The organic EL device emits light generated by the radiation deactivation of the excitons.

### SUMMARY

Embodiments are directed to an organic electroluminescence device and an organic electroluminescence device using the same.

The embodiments may provide a material of an organic EL device driven at a low voltage and having high efficiency and long life, and an organic EL device using the same.

Embodiments provide materials for an electroluminescence (EL) device represented by the following Formula 1.

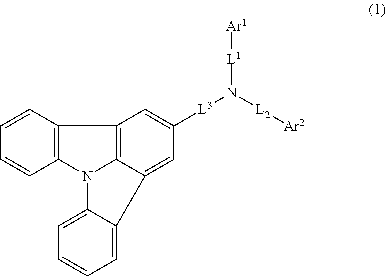

In Formula 1, Ar¹ and Ar² may each independently be a substituted or unsubstituted aryl group, or a substituted or unsubstituted heteroaryl group, L¹, L², and L³ may each independently be a single bond, a substituted or unsubstituted arylene group, or a substituted or unsubstituted heteroarylene group, and at least one of Ar¹, Ar², L¹, L², and L³ is a substituted or unsubstituted heteroaryl group.

The material for an organic EL device may be an amine compound combined at position 2 of an indolo[3,2,1-jk] carbazolyl group with electron tolerance greater than that of a carbazolyl group, and an organic EL device driven at a low voltage and having high efficiency and long life may be manufactured. In addition, at least one of Ar¹, Ar², L¹, L², and L³ may be a substituted or unsubstituted heteroaryl or heteroarylene group, and the material has high hole transporting properties.

In some embodiments, in the above Formula 1, at least one of Ar¹ and Ar² may be a substituted or unsubstituted heteroaryl group, and L¹, L², and L³ may be a single bond or a substituted or unsubstituted arylene group having 6 to 18 ring carbon atoms.

At least one of Ar¹ and Ar² in the above Formula 1 may be the substituted or unsubstituted heteroaryl group in the material for an organic EL device according to an embodiment, may be the amine compound combined at position 2 of the indolo[3,2,1-jk] carbazolyl group via the single bond or the substituted or unsubstituted aryl group having 6 to 18 ring carbon atoms, and the driving at a low voltage, the high efficiency and the long life of the organic EL device may be realized.

In other embodiments, Ar¹ and Ar² in the above Formula 1 may be independently a substituted or unsubstituted aryl group having 6 to 24 ring carbon atoms, a substituted or unsubstituted carbazolyl group, a substituted or unsubstituted dibenzofuryl group, or a substituted or unsubstituted dibenzothienyl group.

At least one of Ar¹, Ar², L¹, L², and L³ may be a substituted or unsubstituted heteroaryl or heteroarylene group, and the material for an organic EL device according to an embodiment may have high hole transporting properties.

In other embodiments, a hole transport material includes the material for an organic EL device described above.

The hole transport material according to an embodiment may be an amine compound combined at position 2 of an indolo[3,2,1-jk] carbazolyl group with electron tolerance greater than that of a carbazolyl group, and an organic EL device driven at a low voltage and having high efficiency and long life may be manufactured. In addition, at least one of Ar¹, Ar², L¹, L², and L³ may be a substituted or unsubstituted heteroaryl group, and the material may have high hole transporting properties.

In still other embodiments, organic EL devices may include the material for an organic

EL device described above in a layer of stacking layers disposed between a anode and an emission layer.

In the organic EL device according to an embodiment, a layer of stacking layers disposed between a anode and an emission layer may be formed by using an amine compound combined at position 2 of an indolo[3,2,1-jk] carbazolyl group with electron tolerance greater than that of a carbazolyl group, and an organic EL device driven at a low voltage and having high efficiency and long life may be manufactured. In addition, at least one of Ar¹, Ar², L¹, L², and L³ may be a substituted or unsubstituted heteroaryl group, and the material has high hole transporting properties.

According to an embodiment, a material for an organic EL device driven at a low voltage and having high efficiency and long life, and an organic EL device using the same may be provided. Particularly, an organic EL device driven at a low voltage and having high efficiency and long life may be manufactured by using the material for an organic EL device in a hole transport layer. In an embodiment, an organic EL device driven at a low voltage and having high efficiency and long life may be manufactured by using an amine compound combined at position 2 of an indolo[3,2,1-jk] carbazolyl group having greater electron tolerance when compared to that of a carbazolyl group as a hole transport material.

### BRIEF DESCRIPTION OF THE DRAWING

Features will become apparent to those of ordinary skill in the art by describing in detail exemplary embodiments with reference to the attached drawing in which:

FIG. 1 illustrates a schematic diagram of an organic EL device 100 according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

An organic EL device driven at a low voltage and having high efficiency and long life may be manufactured by using an amine compound having an indolo[3,2,1-jk] carbazolyl group with electron tolerance greater than that of a carbazolyl group as a hole transport material of an organic EL device. For example, the driving voltage of the organic EL device in a blue emission region and a green emission region may be easily restrained by combining an amine with the indolo[3,2,1-jk] carbazolyl group at the position 2.

The material for an organic EL device according to an embodiment may include an amine compound represented by the following Formula 1.

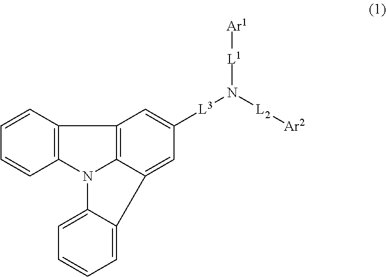

In Formula 1, Ar¹ and Ar² may each independently be a substituted or unsubstituted aryl group or a substituted or unsubstituted heteroaryl group. For example, Ar¹ and Ar² may each independently include an aryl group or a heteroaryl group. L¹, L², and L³ may each independently be a single bond, a substituted or unsubstituted arylene group, or a substituted or unsubstituted heteroarylene group. For example, L¹, L², and L³ may each independently be a single bond, or may each independently include an arylene group or a heteroarylene group. In an implementation, at least one of Ar¹, Ar², L¹, L², and L³ may be or may include a substituted or unsubstituted heteroaryl or heteroarylene group.

The material for an organic EL device according to an embodiment may be an amine compound combined at or bonded to position 2 of an indolo[3,2,1-jk] carbazolyl group with electron tolerance greater than that of a carbazolyl group, and the driving at a low voltage, the high efficiency and the long life of the organic EL device may be realized. In addition, at least one of Ar¹, Ar², L¹, L², and L³ may be a substituted or unsubstituted heteroaryl or heteroarylene group, and the material may have high hole transporting properties.

In the material for an organic EL device according to an embodiment, at least one of Ar¹, Ar², L¹, L², and L³ may preferably a substituted or unsubstituted heteroaryl or heteroarylene group. By introducing the substituted or unsubstituted heteroaryl or heteroarylene group in at least one of Ar¹, Ar², L¹, L², and L³, the material for an organic EL device may be imparted with high hole transporting properties.

In the above Formula 1, examples of the aryl group in the “substituted or unsubstituted aryl group” of Ar¹ and Ar² may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylenyl group, a biphenylenyl group, a pyrenyl group, a benzofluoranthenyl group, a chrysenyl group, etc. In an implementation, the aryl group may be an aryl group having 6 to 24 ring carbon atoms, e.g., the phenyl group, the naphthyl group, the anthracenyl group, the phenanthryl group, the biphenyl group, the terphenyl group, the quaterphenyl group, the fluorenyl group, the triphenylenyl group, the biphenylenyl group, the pyrenyl group, the benzofluoranthenyl group, and the chrysenyl group. By introducing the above-described aryl groups in or on the amine compound having the indolo[3,2,1-jk] carbazolyl group, an organic EL device may be manufactured by a deposition method.

In the above Formula 1, examples of the heteroaryl group in the “substituted or unsubstituted heteroaryl group” of Ar¹ and Ar² may include a benzothiazolyl group, a thiophenyl group, a thienothiophenyl group, a thienothienothiophenyl group, a benzothiophenyl group, a benzofuryl group, a dibenzothiophenyl group, a dibenzofuryl group, a carbazolyl group, a phenoxazyl group, a phenothiazyl group, a pyridyl group, a pyrimidyl group, a triazile group, a quinolinyl group, a quinoxalyl group, etc. In an implementation, the carbazolyl group, the dibenzofuryl group, or the dibenzothienyl group may be included. By introducing the above-described heteroaryl groups in or on the amine compound having the indolo[3,2,1-jk] carbazolyl group, the material for an organic EL device may be imparted with high hole transporting properties.

In the above Formula 1, examples of the arylene group in the “substituted or unsubstituted aryl group” and examples of the heteroarylene group in the “substituted or unsubstituted heteroaryl group” of L¹, L², and L³ may be the same as described above, e.g., may be divalent examples of the aryl and heteroaryl groups described above. In an implementation, L¹, L², and L³ may each independently be a single bond or a substituted or unsubstituted arylene group having 6 to 18 ring carbon atoms. In an implementation, the material for an organic EL device may include an amine compound combined or bound at position 2 of the indolo[3,2,1-jk] carbazolyl group via a single bond or a substituted or unsubstituted arylene group having 6 to 18 ring carbon atoms as a connecting group, and the driving at a low voltage, the high efficiency, and the long life of an organic EL device may be realized.

Examples of the arylene group having 6 to 18 ring carbon atoms of L¹, L², and L³ in Formula 1 may include a phenyl group, a naphthyl group, an anthracenyl group, a phenanthryl group, a biphenyl group, a terphenyl group, a fluorenyl group, a triphenylenyl group, a biphenylenyl group, a pyrenyl group, and a chrysenyl group.

In an implementation, a substituent for the substituted aryl(ene) group or the substituted heteroaryl(ene) group of Ar¹, Ar², L¹, L², and L³ may include, e.g., an aryl group, a heteroaryl group, an alkyl group, an alkoxy group, a triarylsilyl group, or a trialkylsilyl group. As the aryl group and the heteroaryl group, the same groups as described above may be used.

In the above Formula 1, the alkyl group substituent may include an alkyl group having 1 to 30 carbon atoms, e.g., a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, a n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantly group, a 2-ethyldecyl group, a 2-butyldecyl group, a 2-hexyldecyl group, a 2-octyldecyl group, an n-undecyl group, an n-dodecyl group, a 2-ethyldocecyl group, a 2-butyldodecyl group, a 2-hexyldodecyl group, a 2-octyldodecyl group, an n-tridecyl group, an n-tetradecyl group, an n-pentadecyl group, an n-hexadecyl group, a 2-ethylhexadecyl group, 2-butylhexadecyl group, a 2-hexylhexadecyl group, a 2-octylhexadecyl group, an n-heptadecyl group, an n-octadecyl group, an n-nanodecyl group, an n-icosyl group, a 2-ethylicosyl group, a 2-butylicosyl group, a 2-hexylicosyl group, a 2-octylicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc.

In the above Formula 1, the alkoxy group substituent may include an alkoxy group having 1 to 30 carbon atoms, e.g., a methoxy group, an ethoxy group, an n-propoxy group, an i-propoxy group, an n-butoxy group, an s-butoxy group, a t-butoxy group, an i-butoxy group, a 2-ethylbutoxy group, a 3,3-dimethylbutoxy group, an n-pentyloxy group, an i-pentyloxy group, a neopentyloxy group, a t-pentyloxy group, a cyclopentyloxy group, a 1-methylpentyloxy group, a 3-methylpentyloxy group, a 2-ethylpentyloxy group, a 4-methyl-2-pentyloxy group, an n-hexyloxy group, a 1-methylhexyloxy group, a 2-ethylhexyloxy group, a 2-butylhexyloxy group, a cyclohexyloxy group, a 4-methylcyclohexyloxy group, a 4-t-butylcyclohexyloxy group, an n-heptyloxy group, a 1-methylheptyloxy group, a 2,2-dimethylheptyloxy group, a 2-ethylheptyloxy group, 2-butylheptyloxy group, an n-octyloxy group, a t-octyloxy group, a 2-ethyloctyloxy group, a 2-butyloctyloxy group, a 2-hexyloctyloxy group, a 3,7-dimethyloctyloxy group, a cyclooctyloxy group, an n-nonyloxy group, an n-decyloxy group, an adamantyloxy group, a 2-ethyldecyloxy group, a 2-butyldecyloxy group, a 2-hexyldecyloxy group, a 2-octyldecyloxy group, an n-undecyloxy group, an n-dodecyloxy group, a 2-ethyldodecyloxy group, a 2-butyldodecyloxy group, a 2-hexyldodecyloxy group, a 2-octyldodecyloxy group, an n-tridecyloxy group, an n-tetradecyloxy group, an n-pentadecyloxy group, an n-hexadecyloxy group, a 2-ethylhexadecyloxy group, a 2-butylhexadecyloxy group, a 2-hexylhexadecyloxy group, a 2-octylhexadecyloxy group, an n-heptadecyloxy group, an n-octadecyloxy group, an n-nonadecyloxy group, an n-icosyloxy group, a 2-ethylicosyloxy group, a 2-butylicosyloxy group, a 2-hexylicosyloxy group, a 2-octylicosyloxy group, an n-henicosyloxy group, an n-docosyloxy group, an n-tricosyloxy group, an n-tetracosyloxy group, an n-pentacosyloxy group, an n-hexacosyloxy group, an n-heptacosyloxy group, an n-octacosyloxy group, an n-nonacosyloxy group, an n-triacontyloxy group, etc.

In the above Formula 1, the aryl group of the triarylsilyl group substituted for the aryl group or the heteroaryl group used as Ar¹, Ar², L¹, L², and L³ may include the same groups as described above and particularly may include a triphenylsilyl group, etc.

In the above Formula 1, the alkyl group of the trialkylsilyl group substituted for the aryl group or the heteroaryl group used as Ar¹, Ar², L¹, L², and L³ may include the same groups as described above and particularly may include a trimethylsilyl group, a triethylsilyl group, a triisopropylsilyl group, a t-butyldimethylsilyl group, etc.

In an implementation, in the material for an organic EL device, at least one of Ar¹ and Ar² may be a substituted or unsubstituted heteroaryl group, and L¹, L², and L³ may each independently be a single bond or a substituted or unsubstituted arylene group having 6 to 18 ring carbon atoms.

In an implementation, in the material for an organic EL device, at least one of Ar¹ and Ar² may be a substituted or unsubstituted aryl group having 6 to 24 ring carbon atoms, a substituted or unsubstituted carbazolyl group, a substituted or unsubstituted dibenzofuryl group, or a substituted or unsubstituted dibenzothienyl group.

The material for an organic EL device according to an embodiment may have the above-described structure and may have a molecular weight of, e.g., less than or equal to 1,000, for an appropriate application in a vacuum deposition process.

The material for an organic EL device according to an embodiment may use or include an amine compound combined or bound at position 2 of an indolo[3,2,1-jk] carbazolyl group with electron tolerance greater than that of a carbazolyl group as a hole transport material, and an organic EL device driven at a low voltage and having high efficiency and long life may be manufactured.

In an implementation, the material for an organic EL device may include one of the following Compounds 1 to 9.

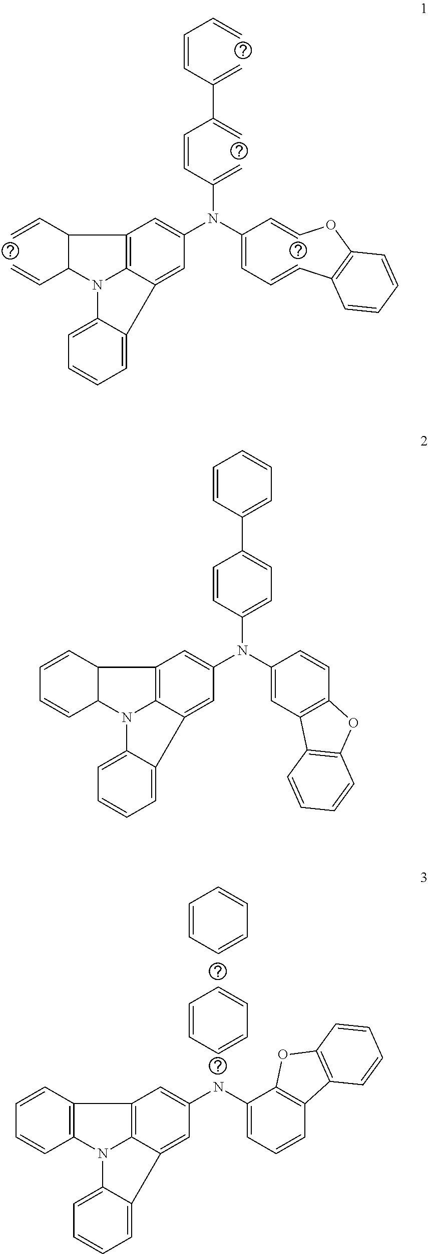

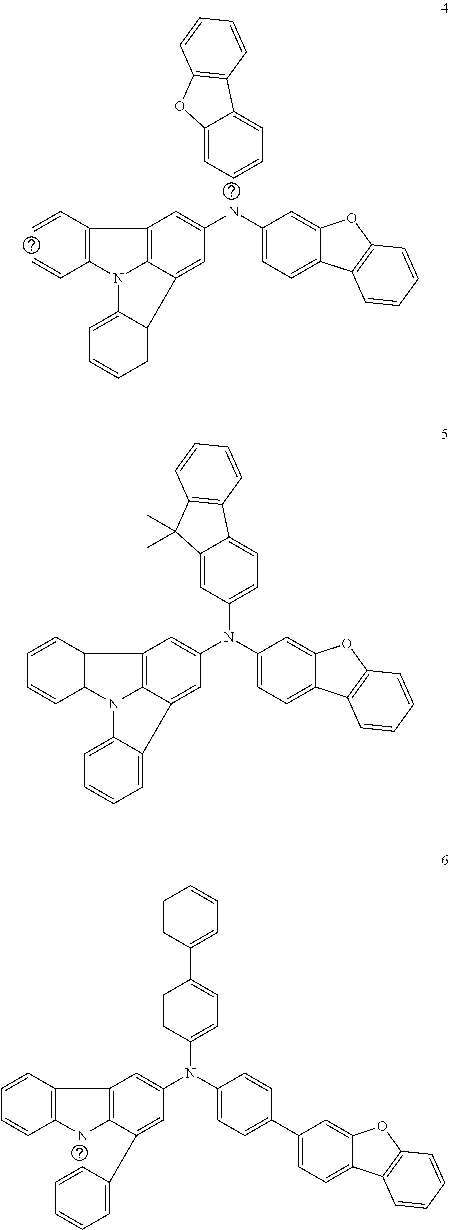

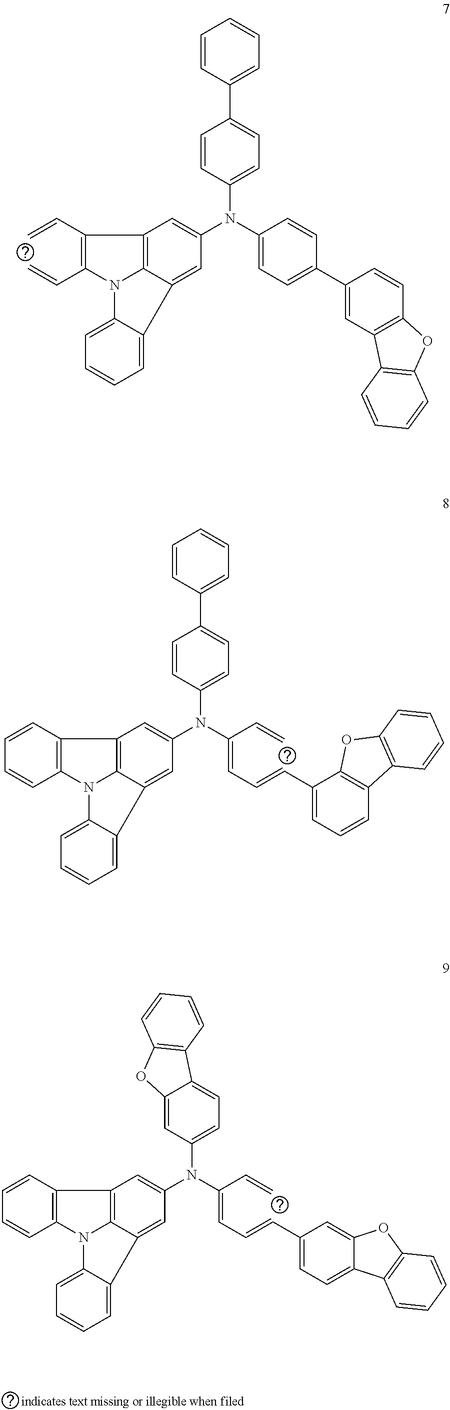 [SMILES: C=Cc1c(C=C)n2c3ccccc3c3cc(N(c4ccc(-c5ccccc5)cc4)c4ccc(-c5ccc6oc7ccccc7c6c5)cc4)cc1c32 ; C=C/C(=C\C=C\c1cccc2c1oc1ccccc12)N(c1ccc(-c2ccccc2)cc1)c1cc2c3ccccc3n3c4ccccc4c(c1)c23 ; C=C/C(=C\C=C\c1ccc2c(c1)oc1ccccc12)N(c1ccc2c(c1)oc1ccccc12)c1cc2c3ccccc3n3c4ccccc4c(c1)c23]

In an implementation, the material for an organic EL device may include one of the following Compounds 10 to 18.

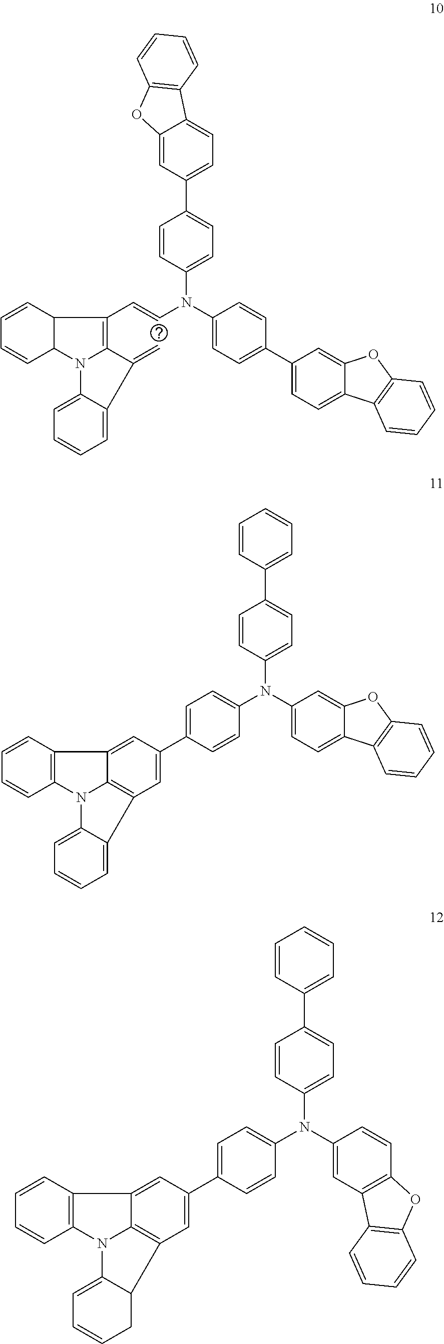 [SMILES: C=c1c2n(c3ccccc13)C1C=CC=CC1C=2/C=C/N(c1ccc(-c2ccc3c(c2)oc2ccccc23)cc1)c1ccc(-c2ccc3c(c2)oc2ccccc23)cc1 ; c1ccc(-c2ccc(N(c3ccc(-c4cc5c6ccccc6n6c7ccccc7c(c4)c56)cc3)c3ccc4c(c3)oc3ccccc34)cc2)cc1 ; C1=CCC2C(=C1)n1c3ccccc3c3cc(-c4ccc(N(c5ccc(-c6ccccc6)cc5)c5ccc6oc7ccccc7c6c5)cc4)cc2c31]

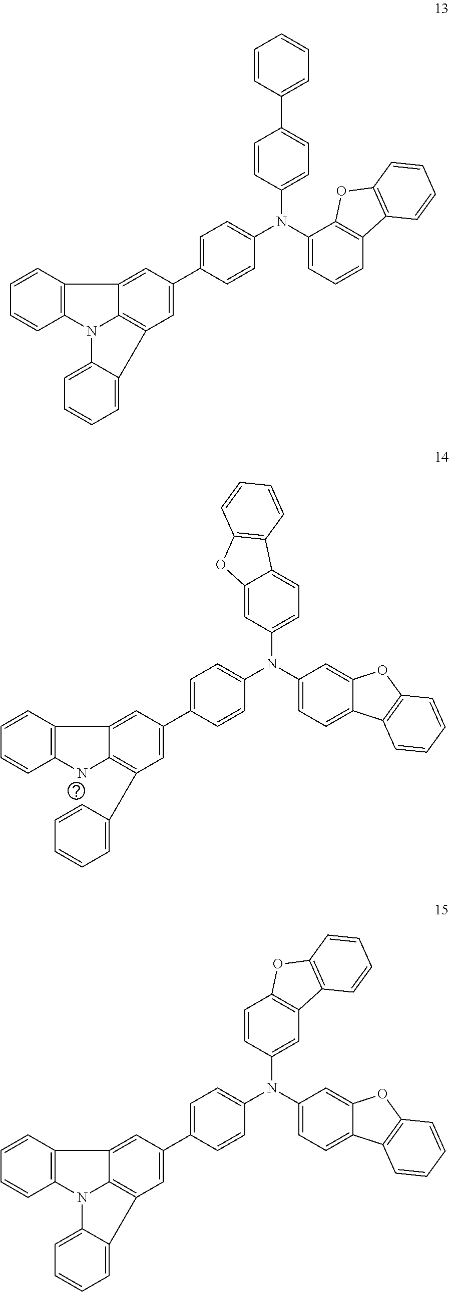 [SMILES: c1ccc(-c2ccc(N(c3ccc(-c4cc5c6ccccc6n6c7ccccc7c(c4)c56)cc3)c3cccc4c3oc3ccccc34)cc2)cc1 ; c1ccc(-c2cc(-c3ccc(N(c4ccc5c(c4)oc4ccccc45)c4ccc5c(c4)oc4ccccc45)cc3)cc3c2[nH]c2ccccc23)cc1 ; c1ccc2c(c1)oc1cc(N(c3ccc(-c4cc5c6ccccc6n6c7ccccc7c(c4)c56)cc3)c3ccc4oc5ccccc5c4c3)ccc12]

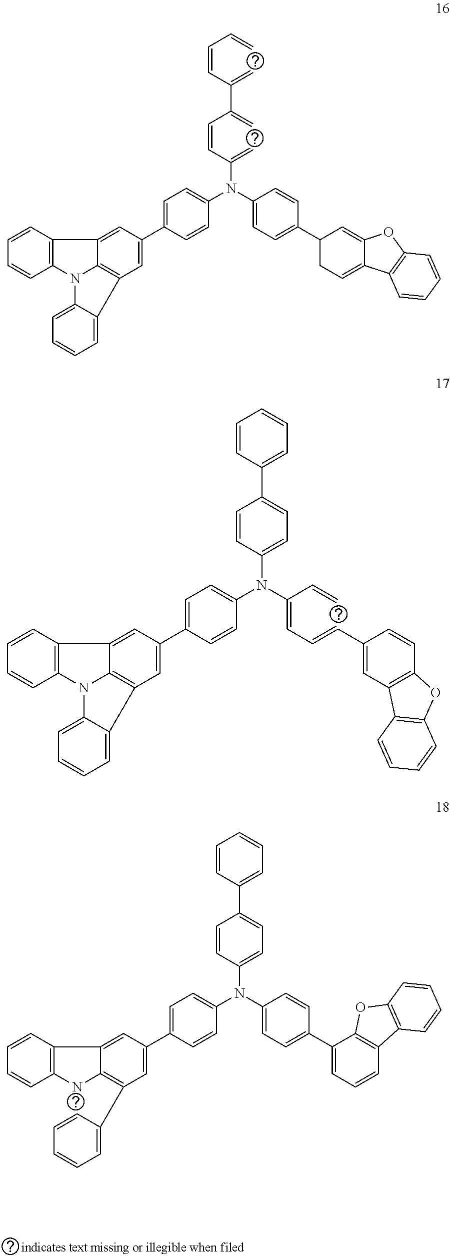 [SMILES: C=C/C=C\C(=C)C(=C)/C=C\C(=C)N(c1ccc(-c2cc3c4ccccc4n4c5ccccc5c(c2)c34)cc1)c1ccc(C2C=c3oc4ccccc4c3=CC2)cc1 ; C=C/C(=C\C=C\c1ccc2oc3ccccc3c2c1)N(c1ccc(-c2ccccc2)cc1)c1ccc(-c2cc3c4ccccc4n4c5ccccc5c(c2)c34)cc1 ; c1ccc(-c2ccc(N(c3ccc(-c4cc(-c5ccccc5)c5[nH]c6ccccc6c5c4)cc3)c3ccc(-c4cccc5c4oc4ccccc45)cc3)cc2)cc1]

In an implementation, the material for an organic EL device may include one of the following Compounds 19 to 27.

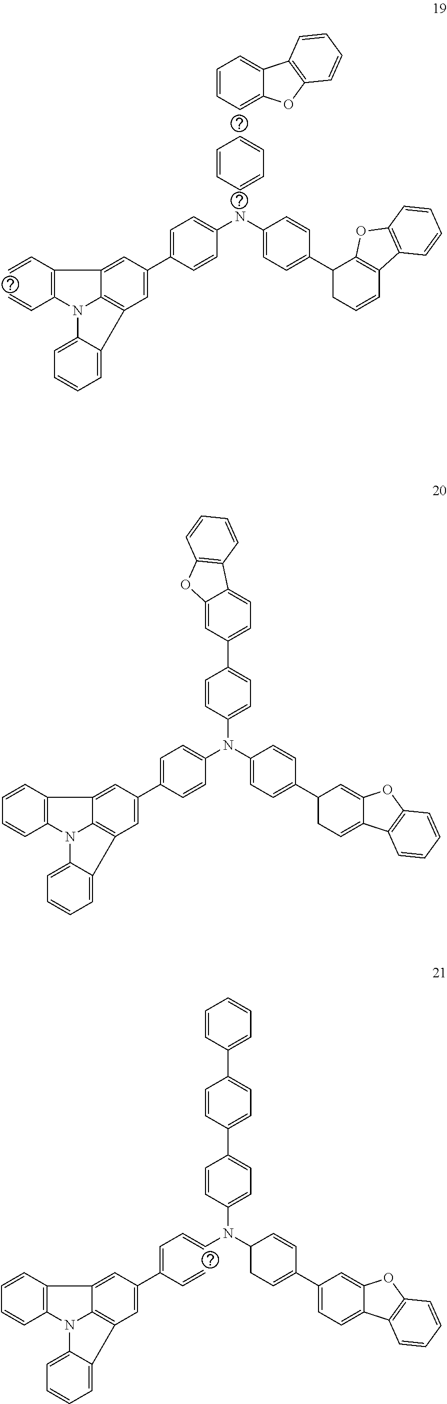

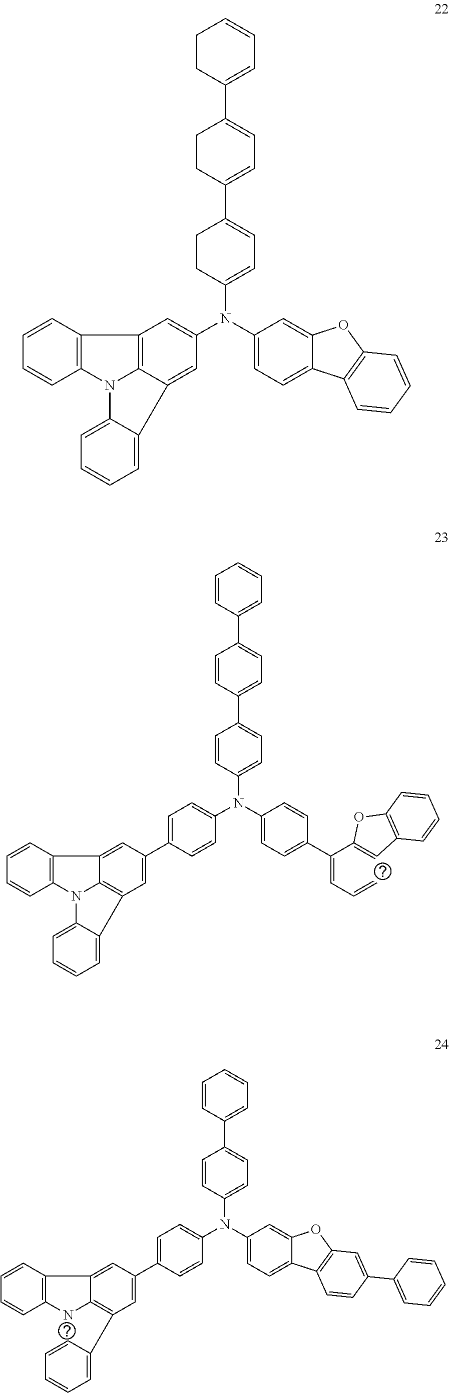 [SMILES: C1=CCCC(C2=CC=C(C3=CC=C(N(c4ccc5c(c4)oc4ccccc45)c4cc5c6ccccc6n6c7ccccc7c(c4)c56)CC3)CC2)=C1 ; C=C/C=C(/c1ccc(N(c2ccc(-c3ccc(-c4ccccc4)cc3)cc2)c2ccc(-c3cc4c5ccccc5n5c6ccccc6c(c3)c45)cc2)cc1)c1cc2ccccc2o1 ; c1ccc(-c2ccc(N(c3ccc(-c4cc(-c5ccccc5)c5[nH]c6ccccc6c5c4)cc3)c3ccc4c(c3)oc3cc(-c5ccccc5)ccc34)cc2)cc1]

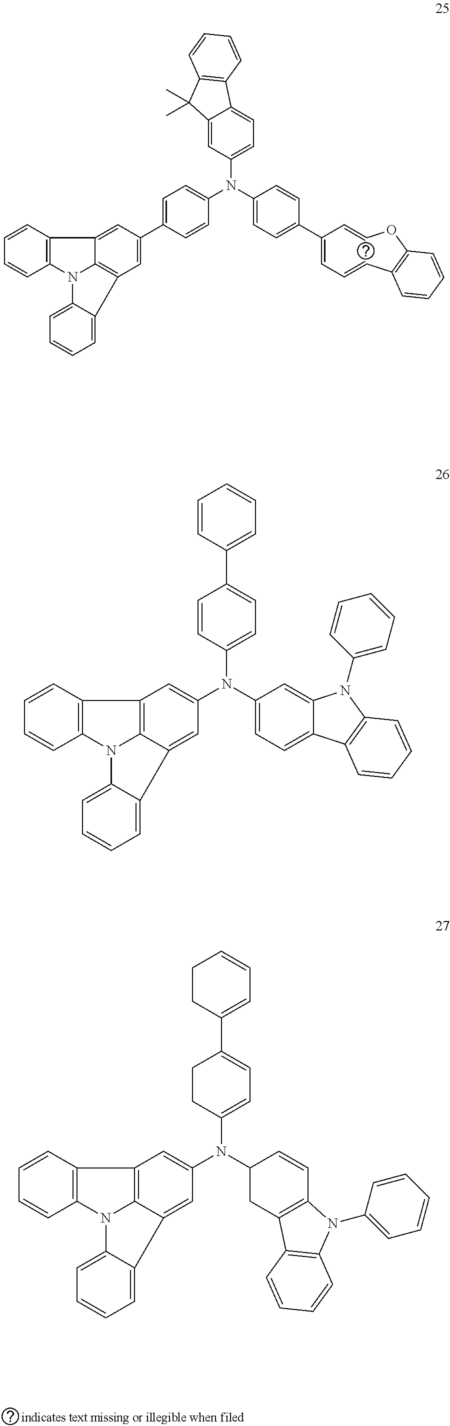 [SMILES: CC1(C)c2ccccc2-c2ccc(N(c3ccc(C4=C/C=C/c5ccccc5O\C=C\4)cc3)c3ccc(-c4cc5c6ccccc6n6c7ccccc7c(c4)c56)cc3)cc21 ; c1ccc(-c2ccc(N(c3cc4c5ccccc5n5c6ccccc6c(c3)c45)c3ccc4c5ccccc5n(-c5ccccc5)c4c3)cc2)cc1 ; C1=CCCC(C2=CC=C(N(c3cc4c5ccccc5n5c6ccccc6c(c3)c45)C3C=Cc4c(c5ccccc5n4-c4ccccc4)C3)CC2)=C1]

In an implementation, the material for an organic EL device may include one of the following Compounds 28 to 36.

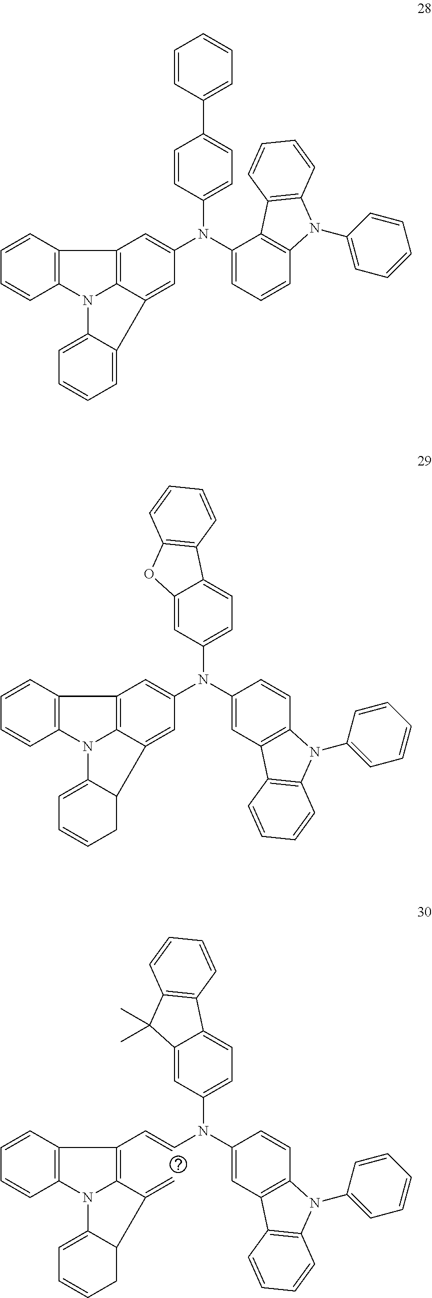 [SMILES: c1ccc(-c2ccc(N(c3cc4c5ccccc5n5c6ccccc6c(c3)c45)c3cccc4c3c3ccccc3n4-c3ccccc3)cc2)cc1 ; C1=CCC2C(=C1)n1c3ccccc3c3cc(N(c4ccc5c(c4)oc4ccccc45)c4ccc5c(c4)c4ccccc4n5-c4ccccc4)cc2c31 ; C=C1c2c(/C=C/N(c3ccc4c(c3)C(C)(C)c3ccccc3-4)c3ccc4c(c3)c3ccccc3n4-c3ccccc3)c3ccccc3n2C2=CC=CCC12]

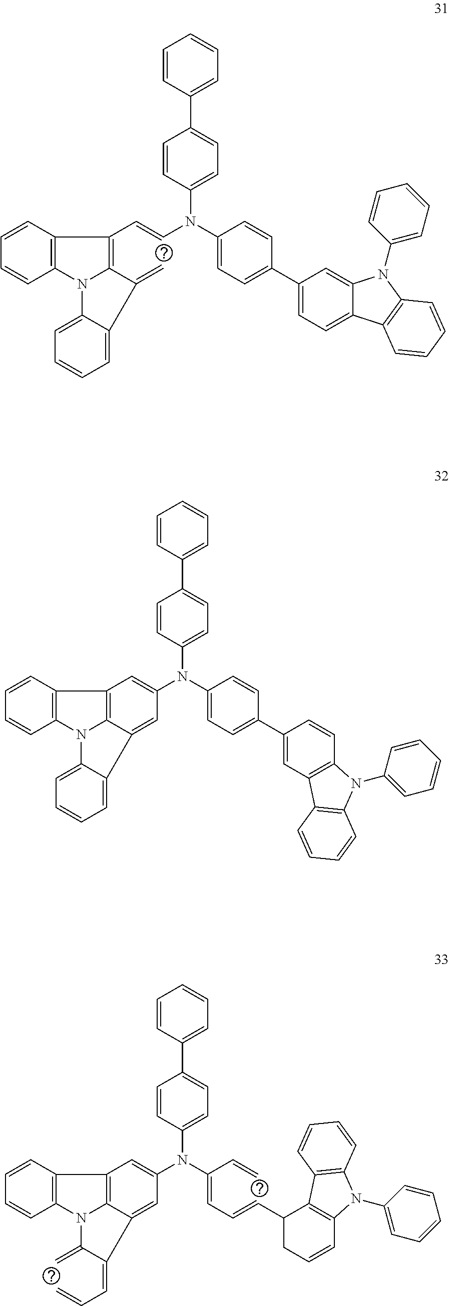 [SMILES: C=c1c2ccccc2n2c1c(/C=C/N(c1ccc(-c3ccccc3)cc1)c1ccc(-c3ccc4c5ccccc5n(-c5ccccc5)c4c3)cc1)c1ccccc12 ; c1ccc(-c2ccc(N(c3ccc(-c4ccc5c(c4)c4ccccc4n5-c4ccccc4)cc3)c3cc4c5ccccc5n5c6ccccc6c(c3)c45)cc2)cc1 ; C=C/C=c1\c(=C)n2c3ccccc3c3cc(N(/C(C=C)=C/C=C/C4CC=Cc5c4c4ccccc4n5-c4ccccc4)c4ccc(-c5ccccc5)cc4)cc1c32]

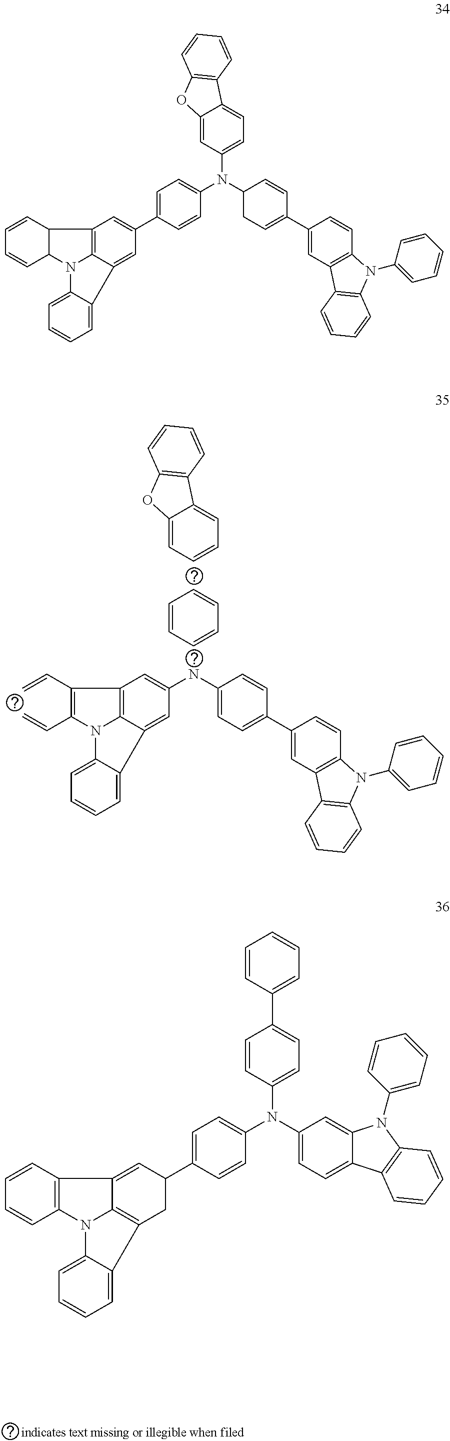

In an implementation, the material for an organic EL device may include one of the following Compounds 37 to 44.

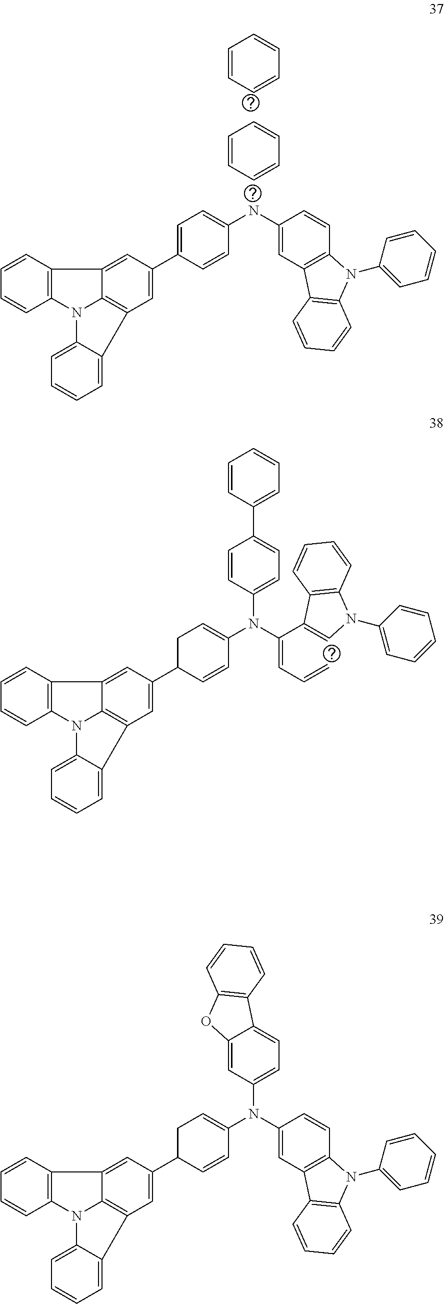

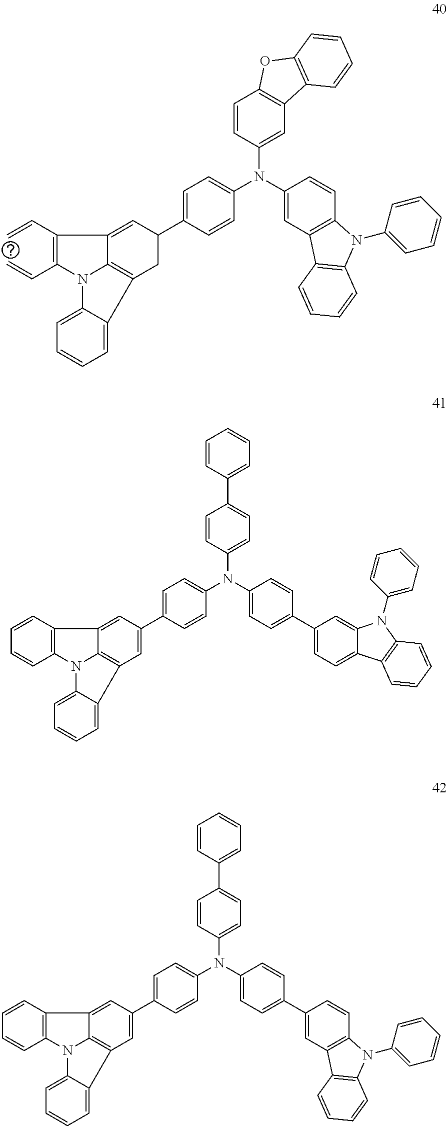 [SMILES: C=Cc1c(C=C)n2c3c(c4ccccc42)CC(c2ccc(N(c4ccc5oc6ccccc6c5c4)c4ccc5c(c4)c4ccccc4n5-c4ccccc4)cc2)C=c13 ; c1ccc(-c2ccc(N(c3ccc(-c4cc5c6ccccc6n6c7ccccc7c(c4)c56)cc3)c3ccc(-c4ccc5c6ccccc6n(-c6ccccc6)c5c4)cc3)cc2)cc1 ; c1ccc(-c2ccc(N(c3ccc(-c4ccc5c(c4)c4ccccc4n5-c4ccccc4)cc3)c3ccc(-c4cc5c6ccccc6n6c7ccccc7c(c4)c56)cc3)cc2)cc1]

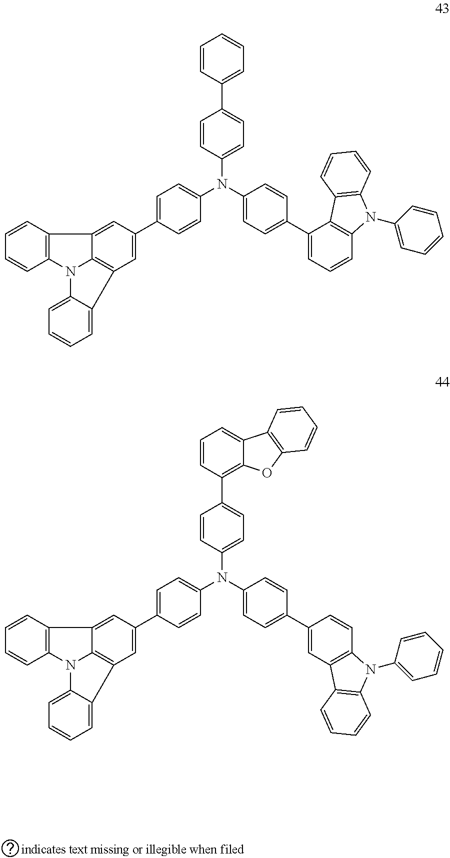 [SMILES: c1ccc(-c2ccc(N(c3ccc(-c4cc5c6ccccc6n6c7ccccc7c(c4)c56)cc3)c3ccc(-c4cccc5c4c4ccccc4n5-c4ccccc4)cc3)cc2)cc1 ; c1ccc(-n2c3ccccc3c3cc(-c4ccc(N(c5ccc(-c6cc7c8ccccc8n8c9ccccc9c(c6)c78)cc5)c5ccc(-c6cccc7c6oc6ccccc67)cc5)cc4)ccc32)cc1]

In an implementation, the material for an organic EL device may include one of the following Compounds 45 to 52.

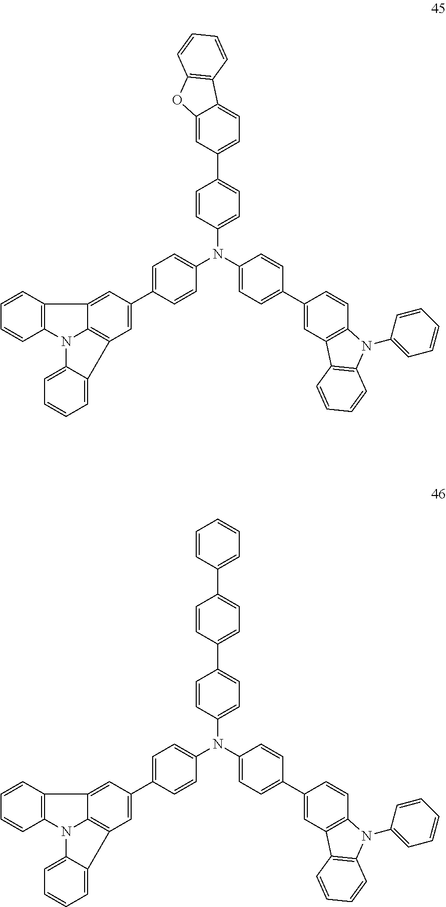 [SMILES: c1ccc(-n2c3ccccc3c3cc(-c4ccc(N(c5ccc(-c6ccc7c(c6)oc6ccccc67)cc5)c5ccc(-c6cc7c8ccccc8n8c9ccccc9c(c6)c78)cc5)cc4)ccc32)cc1 ; c1ccc(-c2ccc(-c3ccc(N(c4ccc(-c5ccc6c(c5)c5ccccc5n6-c5ccccc5)cc4)c4ccc(-c5cc6c7ccccc7n7c8ccccc8c(c5)c67)cc4)cc3)cc2)cc1]

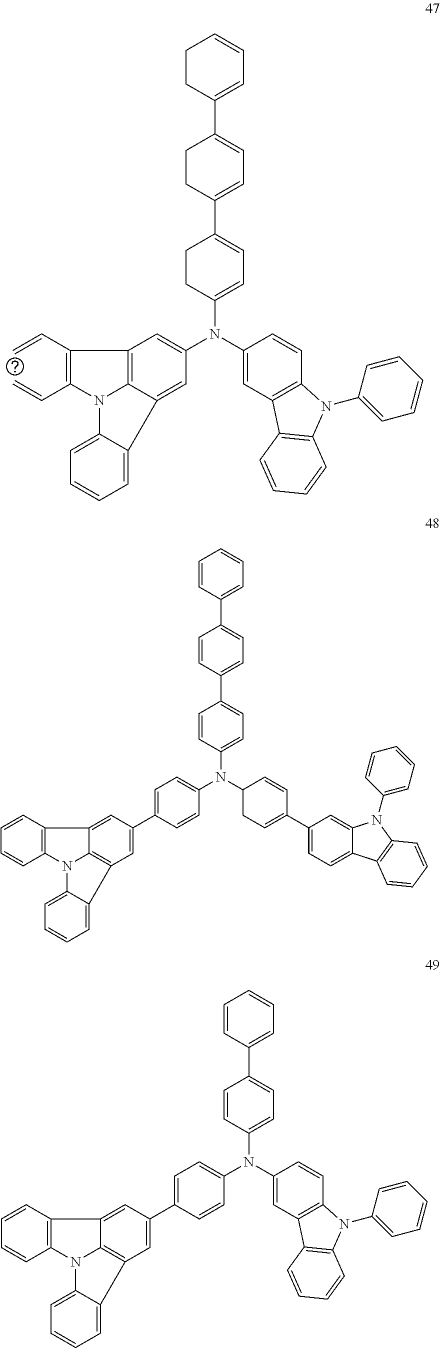 [SMILES: C=Cc1c(C=C)n2c3ccccc3c3cc(N(C4=CC=C(C5=CC=C(C6=CC=CCC6)CC5)CC4)c4ccc5c(c4)c4ccccc4n5-c4ccccc4)cc1c32 ; C1=CC(N(c2ccc(-c3ccc(-c4ccccc4)cc3)cc2)c2ccc(-c3cc4c5ccccc5n5c6ccccc6c(c3)c45)cc2)CC=C1c1ccc2c3ccccc3n(-c3ccccc3)c2c1 ; c1ccc(-c2ccc(N(c3ccc(-c4cc5c6ccccc6n6c7ccccc7c(c4)c56)cc3)c3ccc4c(c3)c3ccccc3n4-c3ccccc3)cc2)cc1]

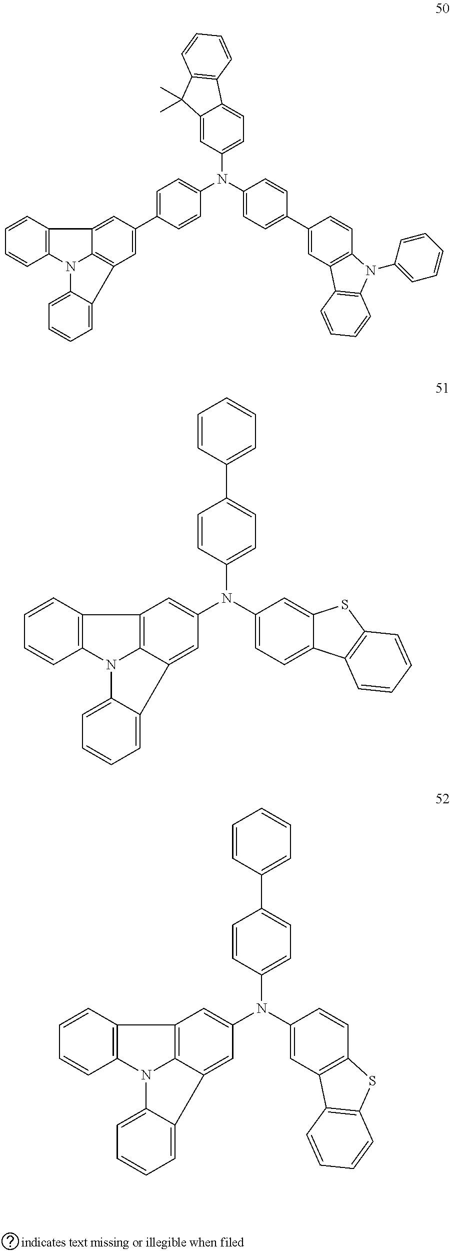 [SMILES: CC1(C)c2ccccc2-c2ccc(N(c3ccc(-c4ccc5c(c4)c4ccccc4n5-c4ccccc4)cc3)c3ccc(-c4cc5c6ccccc6n6c7ccccc7c(c4)c56)cc3)cc21 ; c1ccc(-c2ccc(N(c3ccc4c(c3)sc3ccccc34)c3cc4c5ccccc5n5c6ccccc6c(c3)c45)cc2)cc1 ; c1ccc(-c2ccc(N(c3ccc4sc5ccccc5c4c3)c3cc4c5ccccc5n5c6ccccc6c(c3)c45)cc2)cc1]

In an implementation, the material for an organic EL device may include one of the following Compounds 53 to 61.

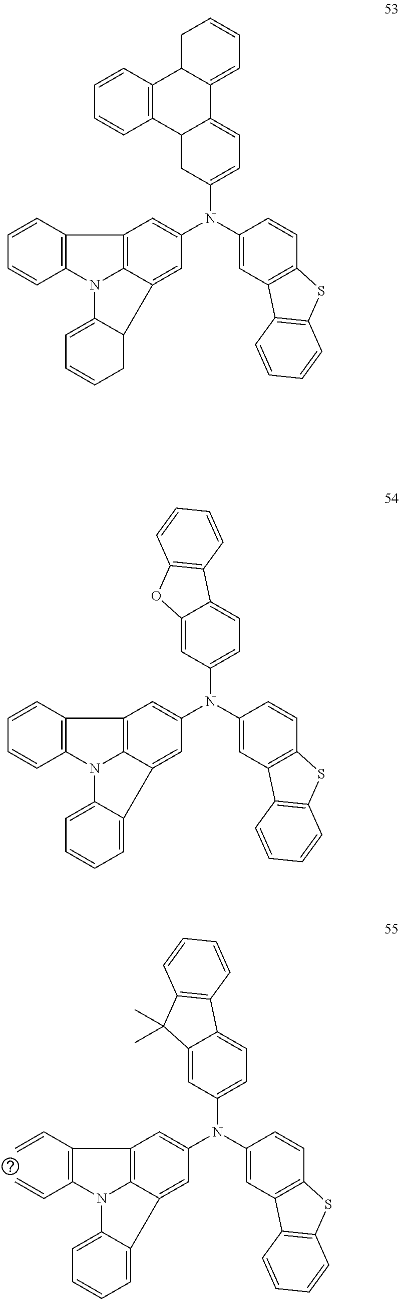 [SMILES: C1=CCC2C(=C1)C1=CC=C(N(c3ccc4sc5ccccc5c4c3)c3cc4c5c(c3)c3ccccc3n5C3=CC=CCC34)CC1c1ccccc12 ; c1ccc2c(c1)oc1cc(N(c3ccc4sc5ccccc5c4c3)c3cc4c5ccccc5n5c6ccccc6c(c3)c45)ccc12 ; C=Cc1c(C=C)n2c3ccccc3c3cc(N(c4ccc5c(c4)C(C)(C)c4ccccc4-5)c4ccc5sc6ccccc6c5c4)cc1c32]

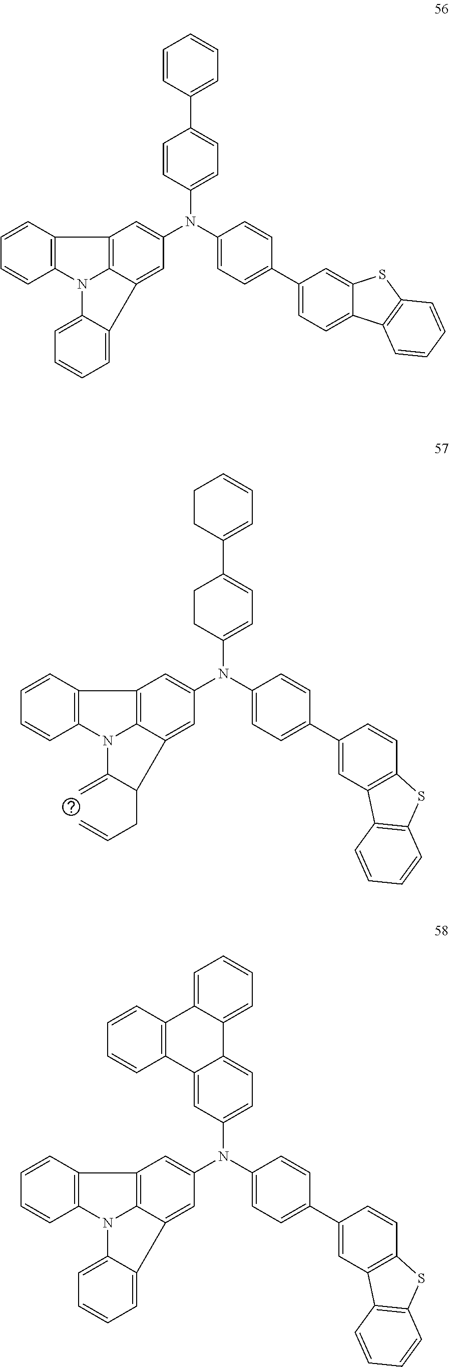 [SMILES: c1ccc(-c2ccc(N(c3ccc(-c4ccc5c(c4)sc4ccccc45)cc3)c3cc4c5ccccc5n5c6ccccc6c(c3)c45)cc2)cc1 ; C=CCC1C(=C)n2c3ccccc3c3cc(N(C4=CC=C(C5=CC=CCC5)CC4)c4ccc(-c5ccc6sc7ccccc7c6c5)cc4)cc1c32 ; c1ccc2c(c1)sc1ccc(-c3ccc(N(c4ccc5c6ccccc6c6ccccc6c5c4)c4cc5c6ccccc6n6c7ccccc7c(c4)c56)cc3)cc12]

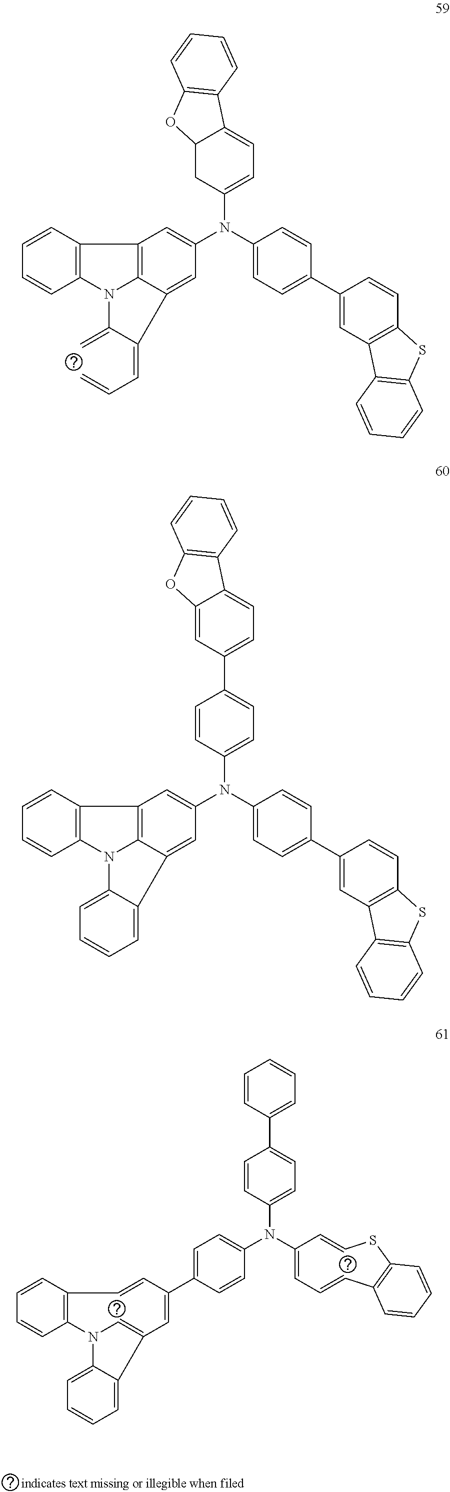 [SMILES: C=C/C=c1\c(=C)n2c3ccccc3c3cc(N(C4=CC=C5c6ccccc6OC5C4)c4ccc(-c5ccc6sc7ccccc7c6c5)cc4)cc1c32 ; c1ccc2c(c1)oc1cc(-c3ccc(N(c4ccc(-c5ccc6sc7ccccc7c6c5)cc4)c4cc5c6ccccc6n6c7ccccc7c(c4)c56)cc3)ccc12 ; C1=C\c2ccccc2S/C=C/C(N(c2ccc(-c3ccccc3)cc2)c2ccc(-c3ccc4ccccc4n4cc(c3)c3ccccc34)cc2)=C\1]

In an implementation, the material for an organic EL device may include one of the following Compounds 62 to 70.

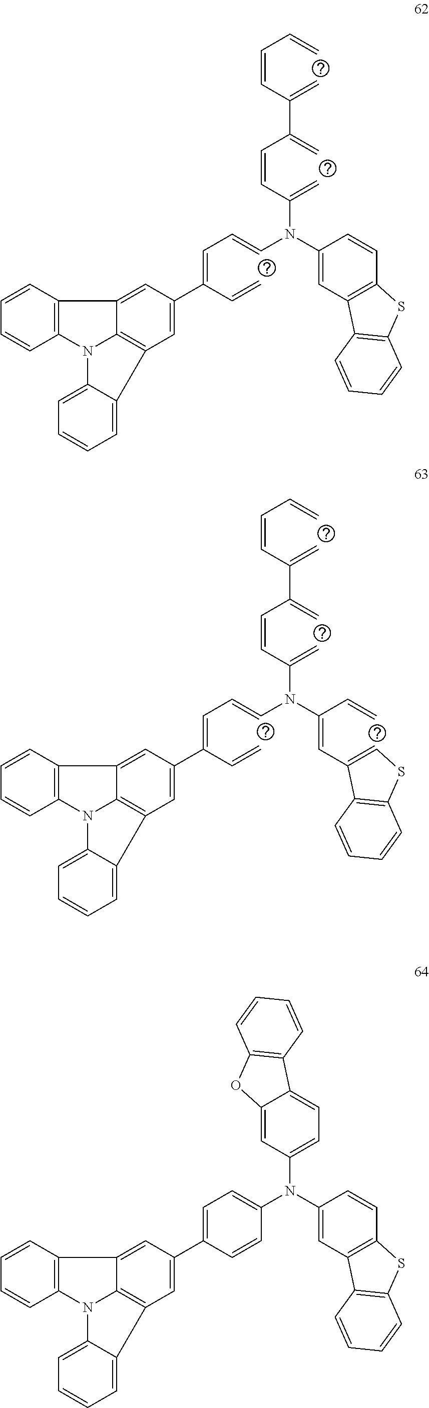 [SMILES: C=C/C=C\C(=C)C(=C)/C=C\C(=C)N(/C=C/C=C(\C=C)c1cc2c3ccccc3n3c4ccccc4c(c1)c23)c1ccc2sc3ccccc3c2c1 ; C=C/C=C\C(=C)C(=C)/C=C\C(=C)N(/C=C/C=C(\C=C)c1cc2c3ccccc3n3c4ccccc4c(c1)c23)/C(C=C)=C/c1csc2ccccc12 ; c1ccc2c(c1)oc1cc(N(c3ccc(-c4cc5c6ccccc6n6c7ccccc7c(c4)c56)cc3)c3ccc4sc5ccccc5c4c3)ccc12]

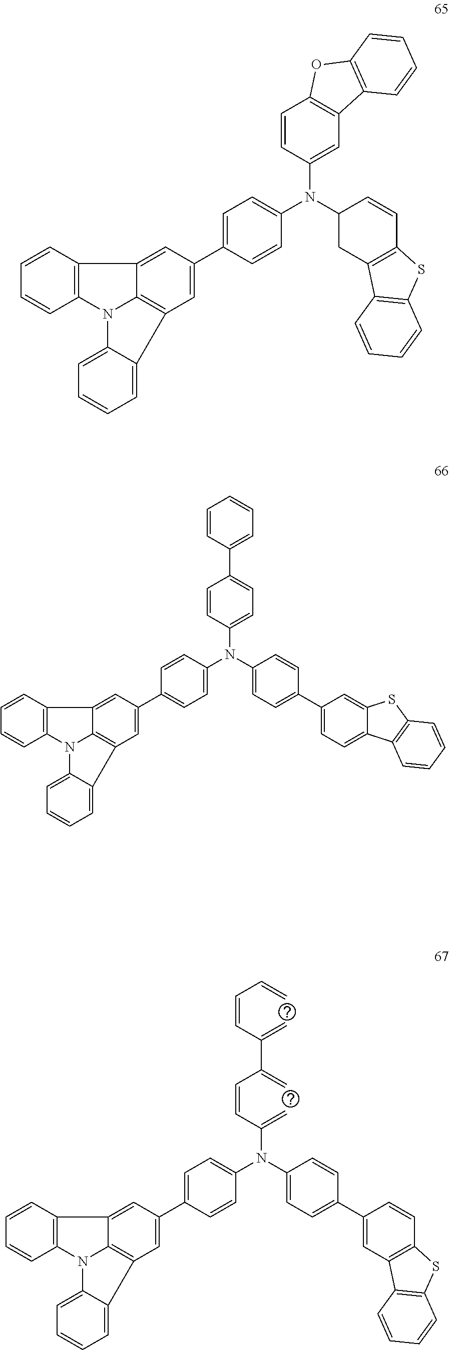 [SMILES: C1=CC(N(c2ccc(-c3cc4c5ccccc5n5c6ccccc6c(c3)c45)cc2)c2ccc3oc4ccccc4c3c2)Cc2c1sc1ccccc21 ; c1ccc(-c2ccc(N(c3ccc(-c4ccc5c(c4)sc4ccccc45)cc3)c3ccc(-c4cc5c6ccccc6n6c7ccccc7c(c4)c56)cc3)cc2)cc1 ; C=C/C=C\C(=C)C(=C)/C=C\C(=C)N(c1ccc(-c2ccc3sc4ccccc4c3c2)cc1)c1ccc(-c2cc3c4ccccc4n4c5ccccc5c(c2)c34)cc1]

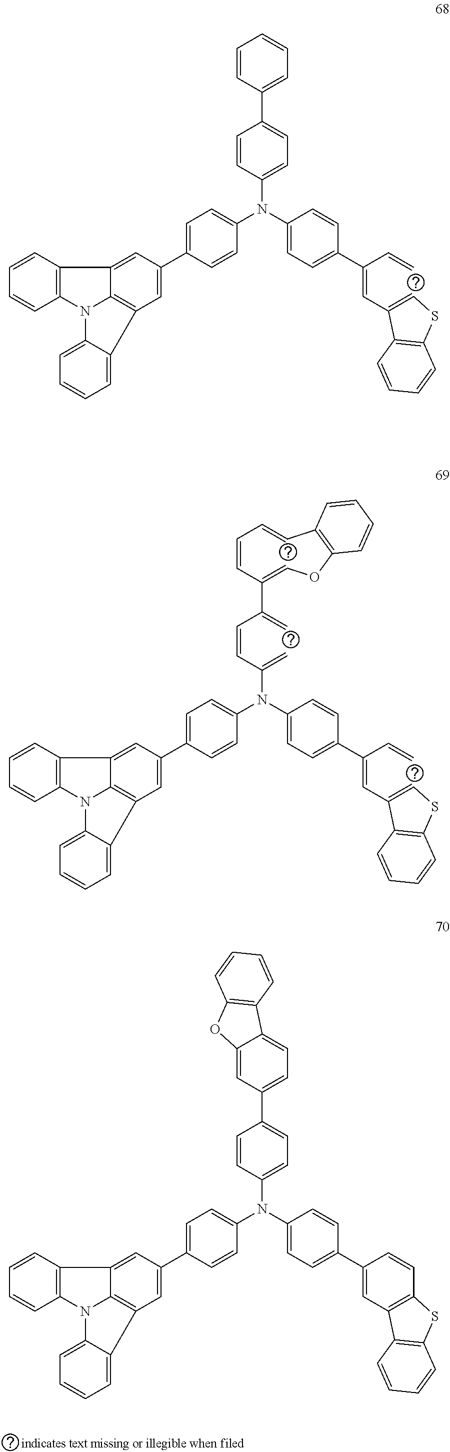 [SMILES: C=C/C(=C\c1csc2ccccc12)c1ccc(N(c2ccc(-c3ccccc3)cc2)c2ccc(-c3cc4c5ccccc5n5c6ccccc6c(c3)c45)cc2)cc1 ; C=C/C(=C\c1csc2ccccc12)c1ccc(N(C(=C)/C=C\C(=C)C2=C\Oc3ccccc3/C=C/C=C\2)c2ccc(-c3cc4c5ccccc5n5c6ccccc6c(c3)c45)cc2)cc1 ; c1ccc2c(c1)oc1cc(-c3ccc(N(c4ccc(-c5ccc6sc7ccccc7c6c5)cc4)c4ccc(-c5cc6c7ccccc7n7c8ccccc8c(c5)c67)cc4)cc3)ccc12]

In an implementation, the material for an organic EL device may include one of the following Compounds 71 to 79.

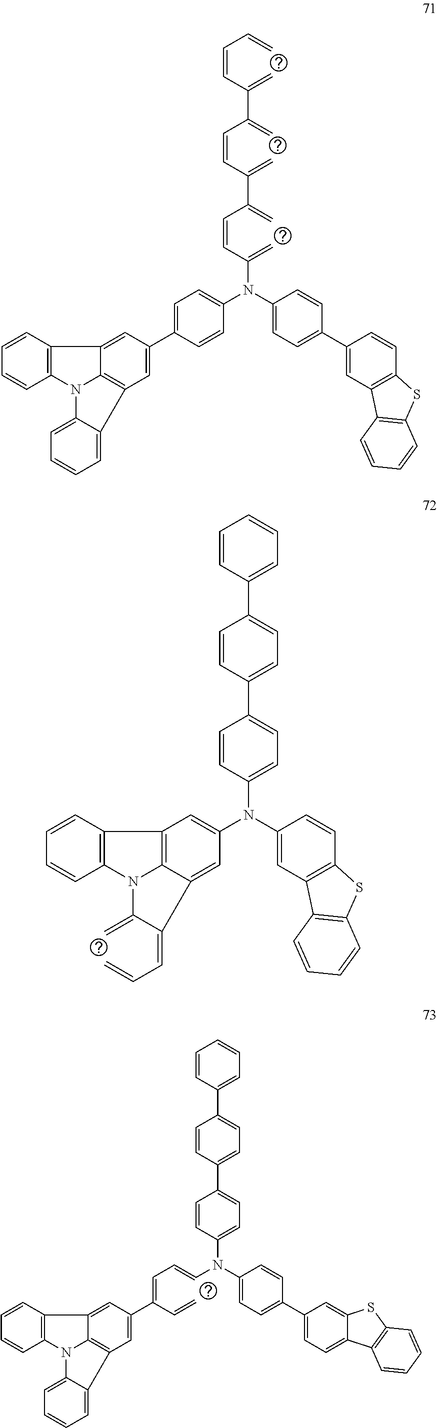 [SMILES: C=C/C=C\C(=C)C(=C)/C=C\C(=C)C(=C)/C=C\C(=C)N(c1ccc(-c2ccc3sc4ccccc4c3c2)cc1)c1ccc(-c2cc3c4ccccc4n4c5ccccc5c(c2)c34)cc1 ; C=C/C=c1\c(=C)n2c3ccccc3c3cc(N(c4ccc(-c5ccc(-c6ccccc6)cc5)cc4)c4ccc5sc6ccccc6c5c4)cc1c32 ; C=C/C(=C\C=C\N(c1ccc(-c2ccc(-c3ccccc3)cc2)cc1)c1ccc(-c2ccc3c(c2)sc2ccccc23)cc1)c1cc2c3ccccc3n3c4ccccc4c(c1)c23]

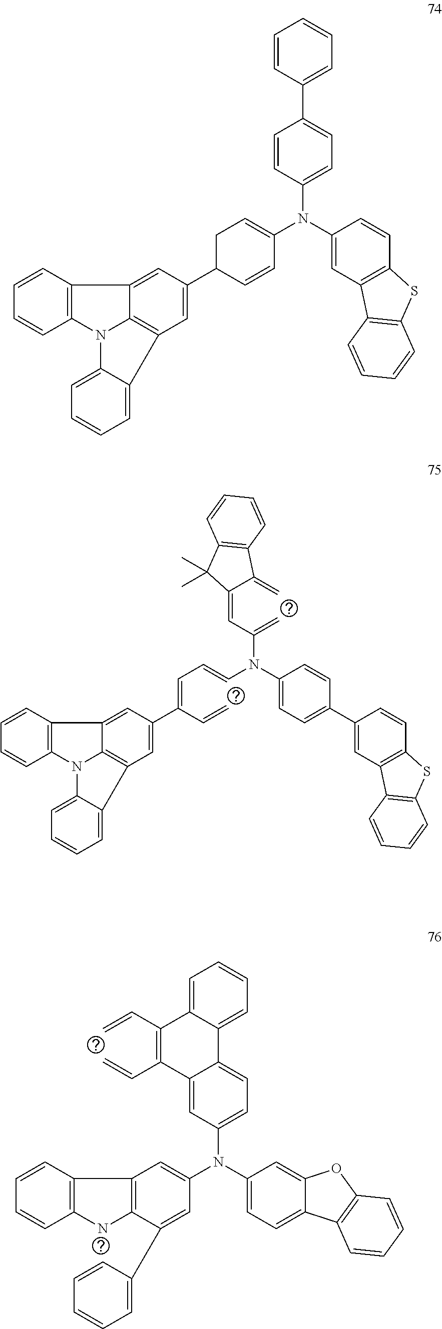 [SMILES: C1=CC(c2cc3c4ccccc4n4c5ccccc5c(c2)c34)CC=C1N(c1ccc(-c2ccccc2)cc1)c1ccc2sc3ccccc3c2c1 ; C=C/C(=C\C=C\N(C(=C)/C=C1\C(=C)c2ccccc2C1(C)C)c1ccc(-c2ccc3sc4ccccc4c3c2)cc1)c1cc2c3ccccc3n3c4ccccc4c(c1)c23 ; C=Cc1c(C=C)c2cc(N(c3ccc4c(c3)oc3ccccc34)c3cc(-c4ccccc4)c4[nH]c5ccccc5c4c3)ccc2c2ccccc12]

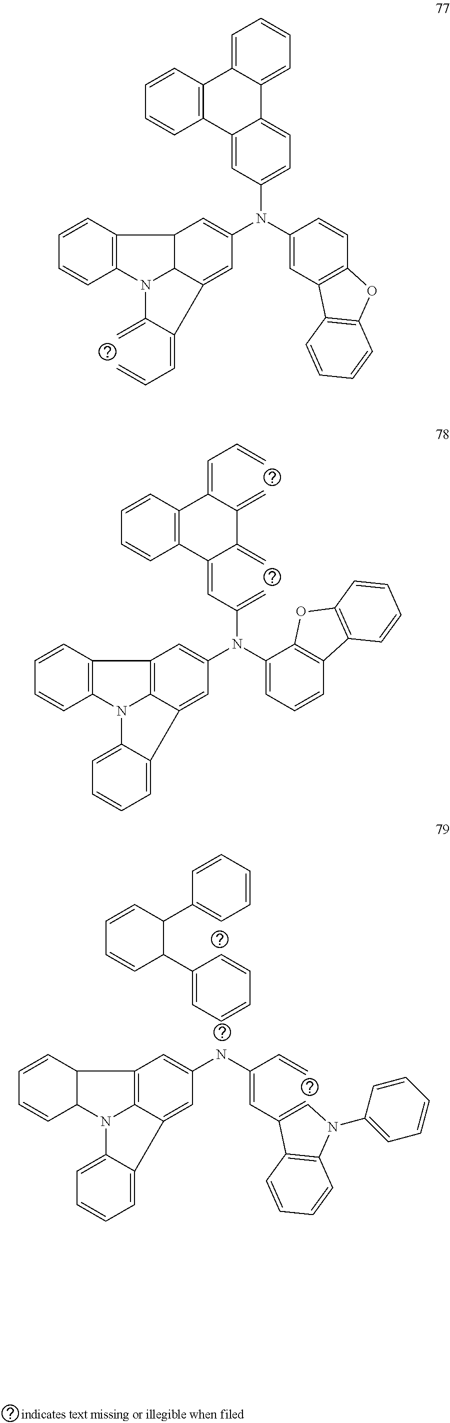

In an implementation, the material for an organic EL device may include one of the following Compounds 80 to 88.

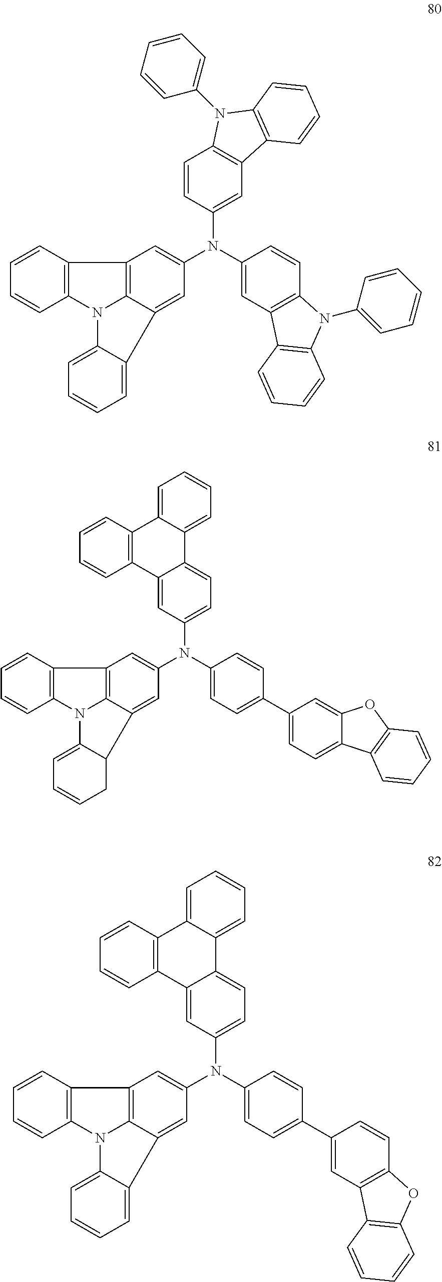 [SMILES: c1ccc(-n2c3ccccc3c3cc(N(c4ccc5c(c4)c4ccccc4n5-c4ccccc4)c4cc5c6ccccc6n6c7ccccc7c(c4)c56)ccc32)cc1 ; C1=CCC2C(=C1)n1c3ccccc3c3cc(N(c4ccc(-c5ccc6c(c5)oc5ccccc56)cc4)c4ccc5c6ccccc6c6ccccc6c5c4)cc2c31 ; c1ccc2c(c1)oc1ccc(-c3ccc(N(c4ccc5c6ccccc6c6ccccc6c5c4)c4cc5c6ccccc6n6c7ccccc7c(c4)c56)cc3)cc12]

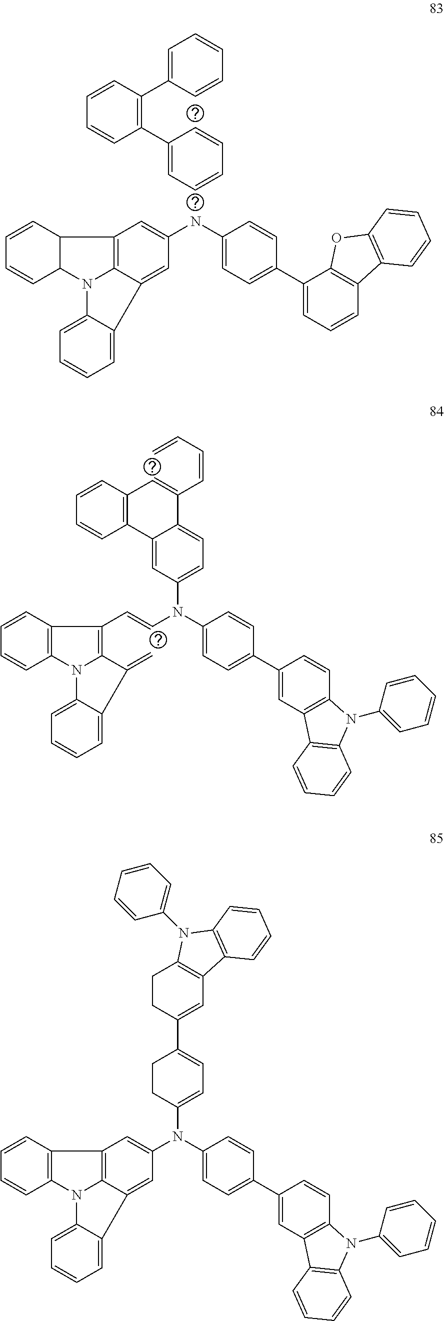

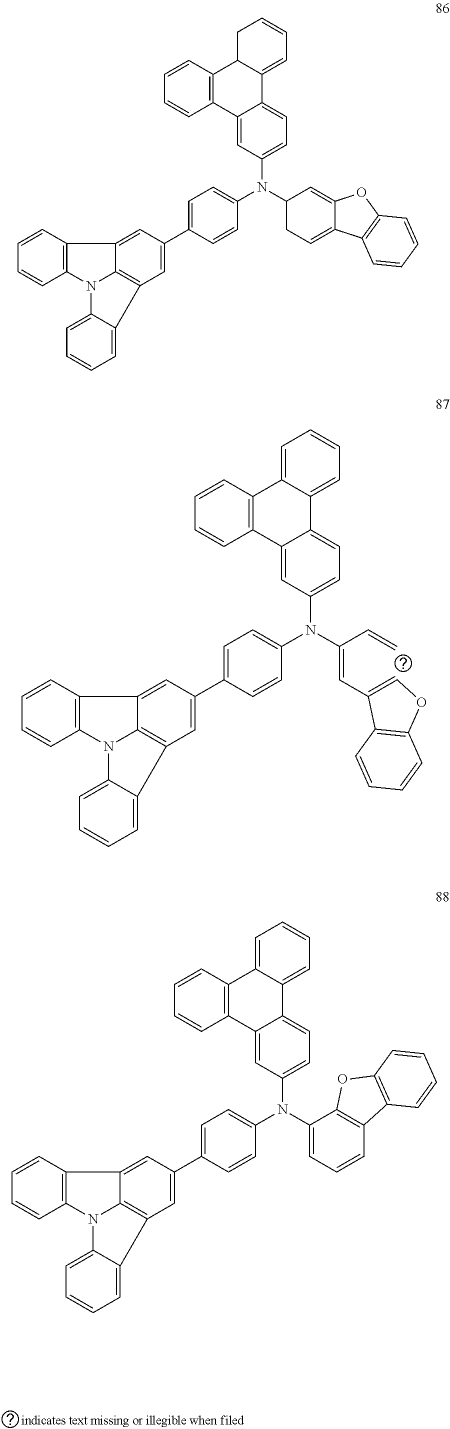 [SMILES: C1=CCC2C(=C1)c1ccc(N(c3ccc(-c4cc5c6ccccc6n6c7ccccc7c(c4)c56)cc3)C3C=c4oc5ccccc5c4=CC3)cc1-c1ccccc12 ; C=C/C(=C\c1coc2ccccc12)N(c1ccc(-c2cc3c4ccccc4n4c5ccccc5c(c2)c34)cc1)c1ccc2c3ccccc3c3ccccc3c2c1 ; c1ccc2c(c1)oc1c(N(c3ccc(-c4cc5c6ccccc6n6c7ccccc7c(c4)c56)cc3)c3ccc4c5ccccc5c5ccccc5c4c3)cccc12]

In an implementation, the material for an organic EL device may include one of the following Compounds 89 to 95.

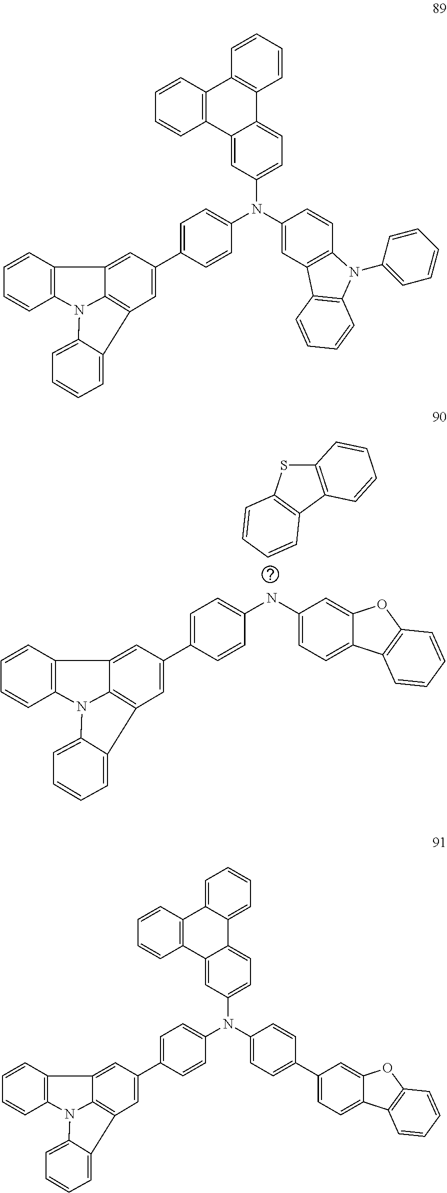 [SMILES: c1ccc(-n2c3ccccc3c3cc(N(c4ccc(-c5cc6c7ccccc7n7c8ccccc8c(c5)c67)cc4)c4ccc5c6ccccc6c6ccccc6c5c4)ccc32)cc1 ; c1ccc2c(c1)sc1ccccc12 ; c1ccc2c(c1)oc1cc(Nc3ccc(-c4cc5c6ccccc6n6c7ccccc7c(c4)c56)cc3)ccc12 ; c1ccc2c(c1)oc1cc(-c3ccc(N(c4ccc(-c5cc6c7ccccc7n7c8ccccc8c(c5)c67)cc4)c4ccc5c6ccccc6c6ccccc6c5c4)cc3)ccc12]

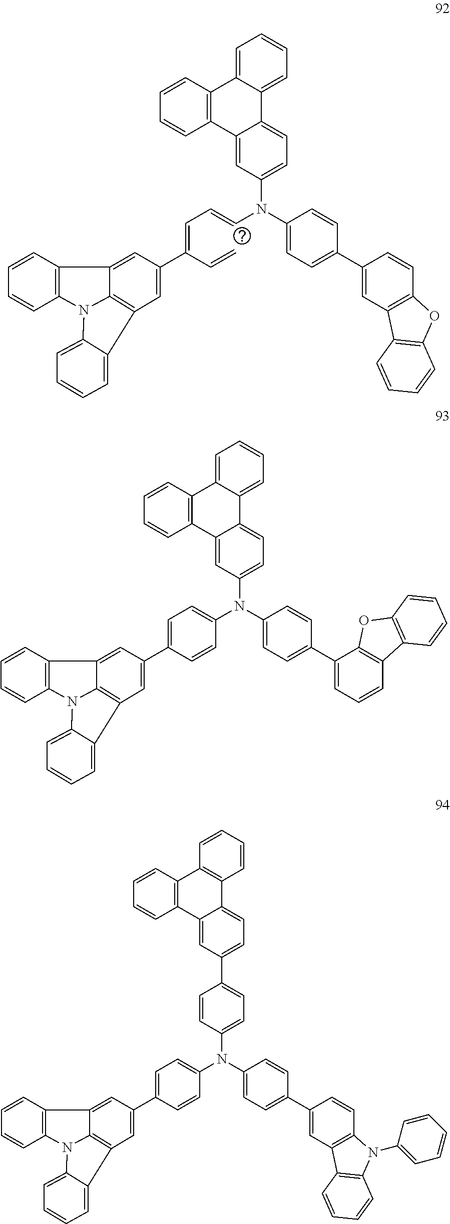 [SMILES: C=C/C(=C\C=C\N(c1ccc(-c2ccc3oc4ccccc4c3c2)cc1)c1ccc2c3ccccc3c3ccccc3c2c1)c1cc2c3ccccc3n3c4ccccc4c(c1)c23 ; c1ccc2c(c1)oc1c(-c3ccc(N(c4ccc(-c5cc6c7ccccc7n7c8ccccc8c(c5)c67)cc4)c4ccc5c6ccccc6c6ccccc6c5c4)cc3)cccc12 ; c1ccc(-n2c3ccccc3c3cc(-c4ccc(N(c5ccc(-c6ccc7c8ccccc8c8ccccc8c7c6)cc5)c5ccc(-c6cc7c8ccccc8n8c9ccccc9c(c6)c78)cc5)cc4)ccc32)cc1]

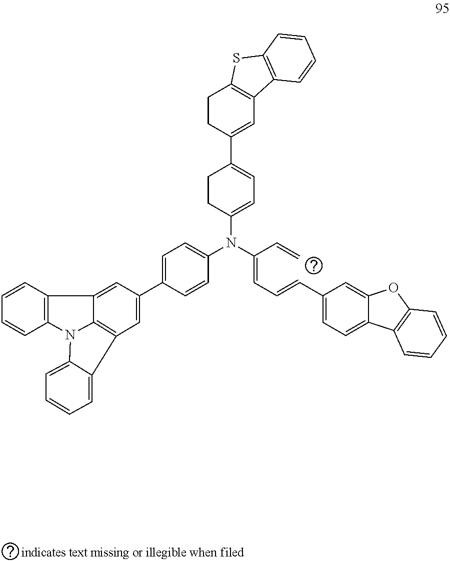 [SMILES: C=C/C(=C\C=C\c1ccc2c(c1)oc1ccccc12)N(C1=CC=C(C2=Cc3c(sc4ccccc34)CC2)CC1)c1ccc(-c2cc3c4ccccc4n4c5ccccc5c(c2)c34)cc1]

In an implementation, the material for an organic EL device may include one of the following Compounds 96 to 104.

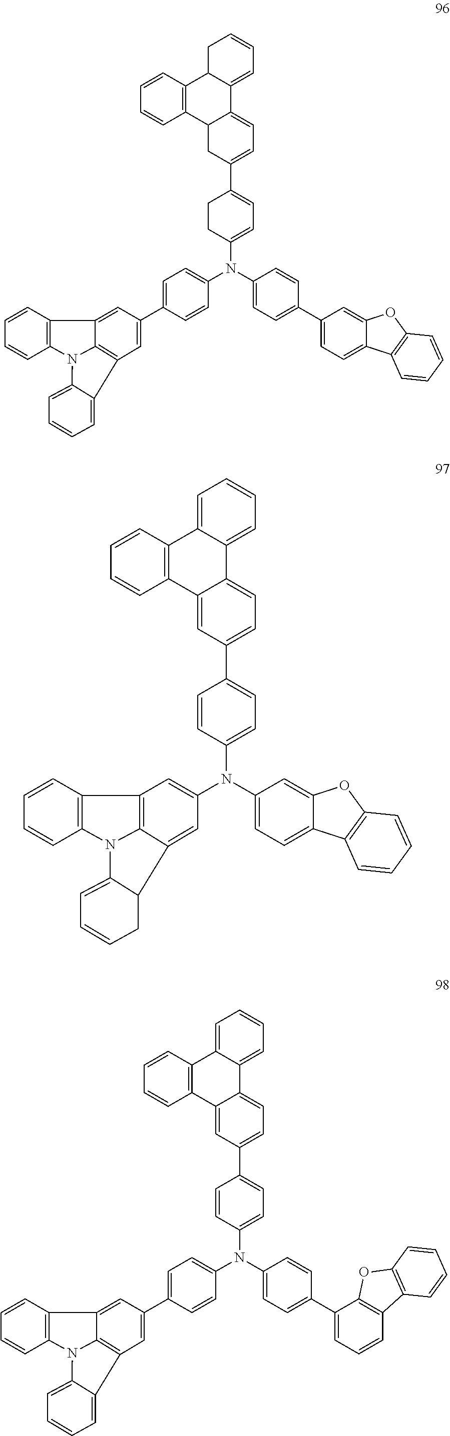 [SMILES: C1=CCC2C(=C1)C1=CC=C(C3=CC=C(N(c4ccc(-c5ccc6c(c5)oc5ccccc56)cc4)c4ccc(-c5cc6c7ccccc7n7c8ccccc8c(c5)c67)cc4)CC3)CC1c1ccccc12 ; C1=CCC2C(=C1)n1c3ccccc3c3cc(N(c4ccc(-c5ccc6c7ccccc7c7ccccc7c6c5)cc4)c4ccc5c(c4)oc4ccccc45)cc2c31 ; c1ccc2c(c1)oc1c(-c3ccc(N(c4ccc(-c5ccc6c7ccccc7c7ccccc7c6c5)cc4)c4ccc(-c5cc6c7ccccc7n7c8ccccc8c(c5)c67)cc4)cc3)cccc12]

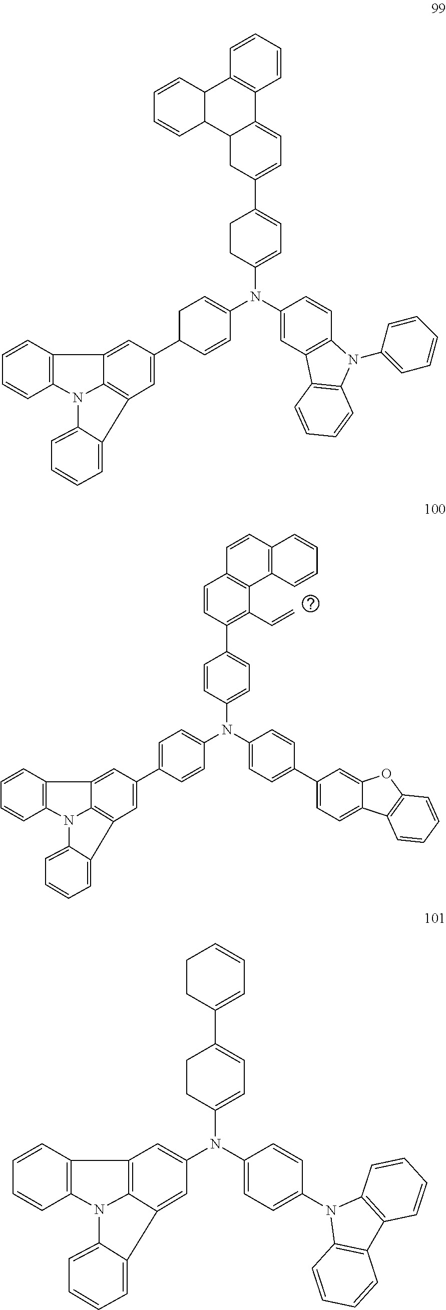 [SMILES: C1=CC2c3ccccc3C3=CC=C(C4=CC=C(N(C5=CCC(c6cc7c8ccccc8n8c9ccccc9c(c6)c78)C=C5)c5ccc6c(c5)c5ccccc5n6-c5ccccc5)CC4)CC3C2C=C1 ; C=Cc1c(-c2ccc(N(c3ccc(-c4ccc5c(c4)oc4ccccc45)cc3)c3ccc(-c4cc5c6ccccc6n6c7ccccc7c(c4)c56)cc3)cc2)ccc2ccc3ccccc3c12 ; C1=CCCC(C2=CC=C(N(c3ccc(-n4c5ccccc5c5ccccc54)cc3)c3cc4c5ccccc5n5c6ccccc6c(c3)c45)CC2)=C1]

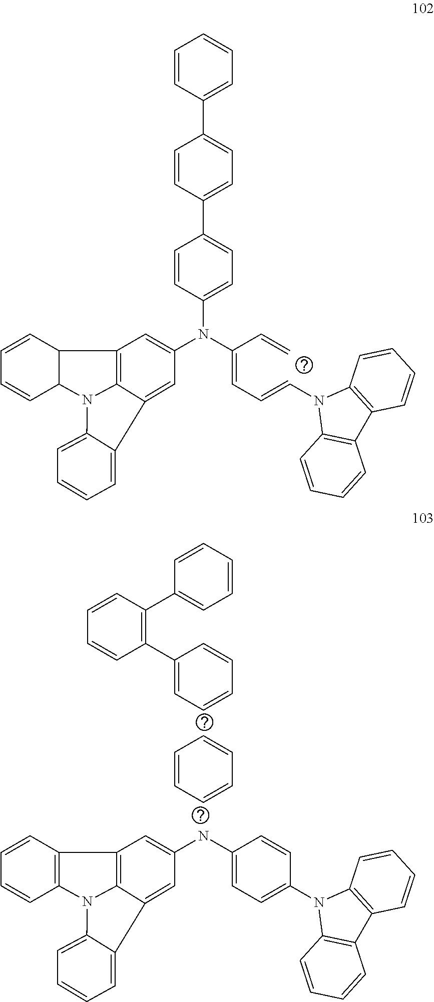

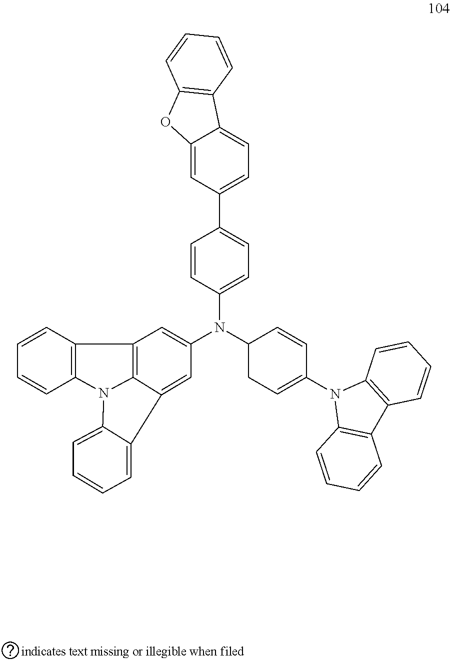 [SMILES: C1=CC(N(c2ccc(-c3ccc4c(c3)oc3ccccc34)cc2)c2cc3c4ccccc4n4c5ccccc5c(c2)c34)CC=C1n1c2ccccc2c2ccccc21]

In an implementation, the material for an organic EL device may include one of the following Compounds 105 to 113.

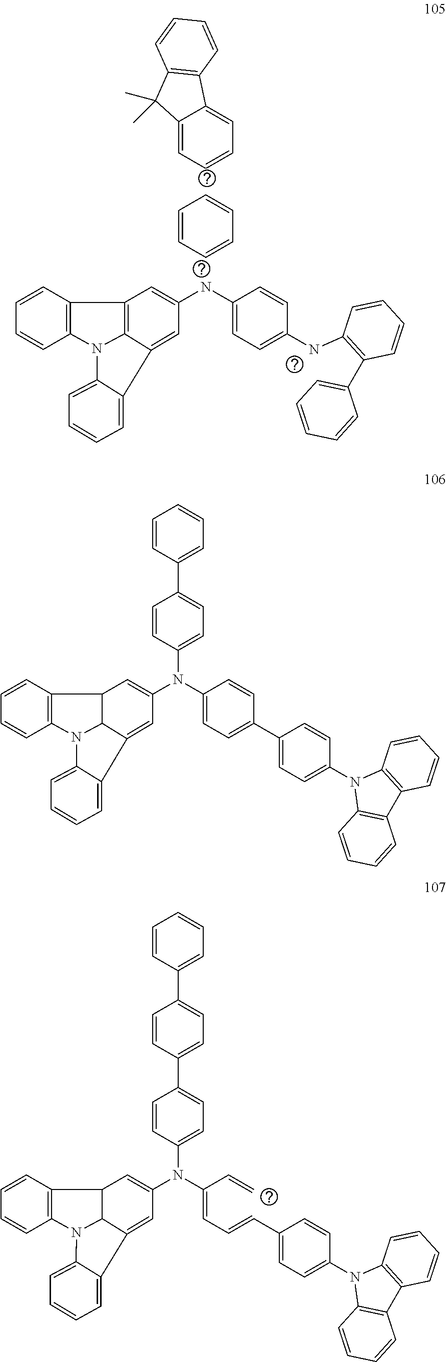

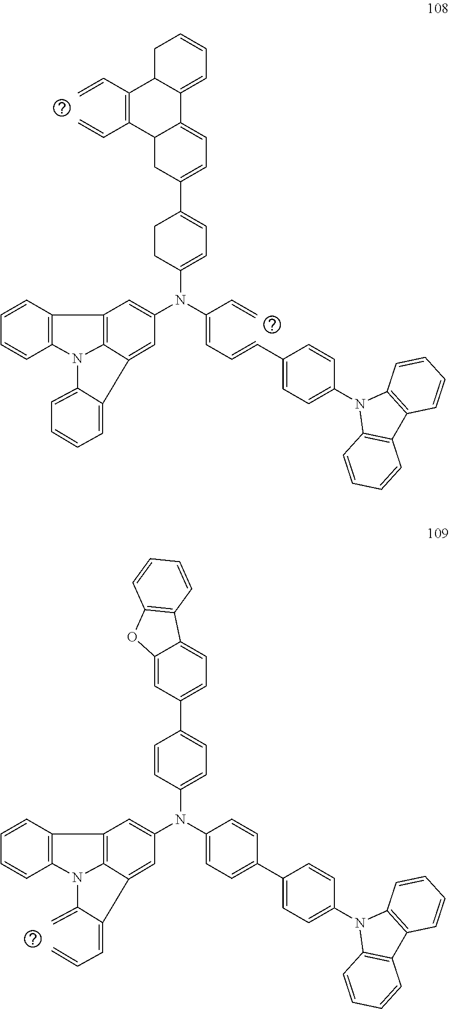 [SMILES: C=CC1=C(C=C)C2CC(C3=CC=C(N(/C(C=C)=C/C=C/c4ccc(-n5c6ccccc6c6ccccc65)cc4)c4cc5c6ccccc6n6c7ccccc7c(c4)c56)CC3)=CC=C2C2=CC=CCC21 ; C=C/C=c1\c(=C)n2c3ccccc3c3cc(N(c4ccc(-c5ccc(-n6c7ccccc7c7ccccc76)cc5)cc4)c4ccc(-c5ccc6c(c5)oc5ccccc56)cc4)cc1c32]

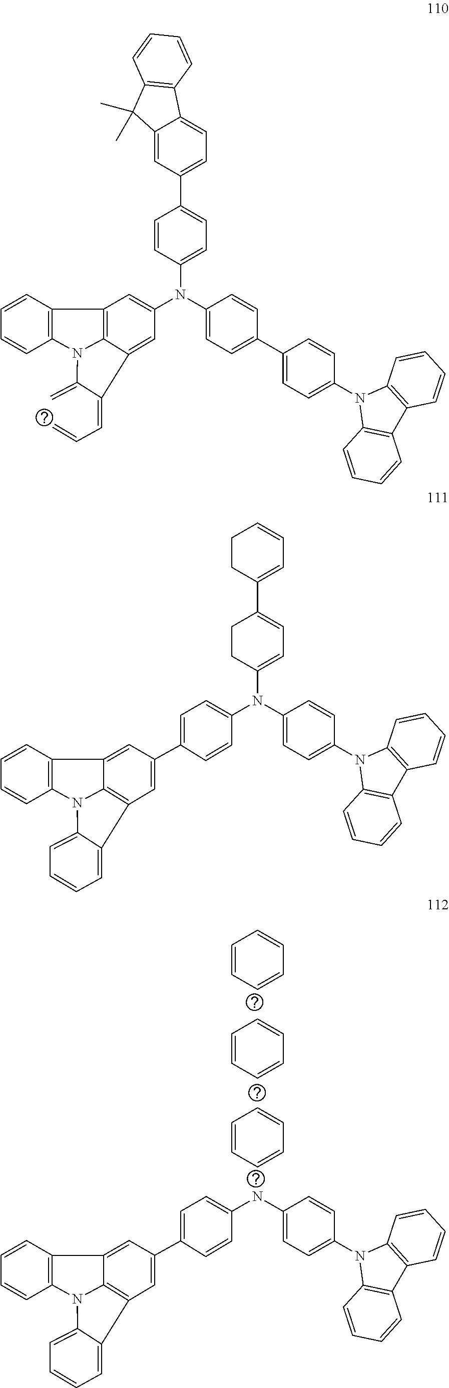

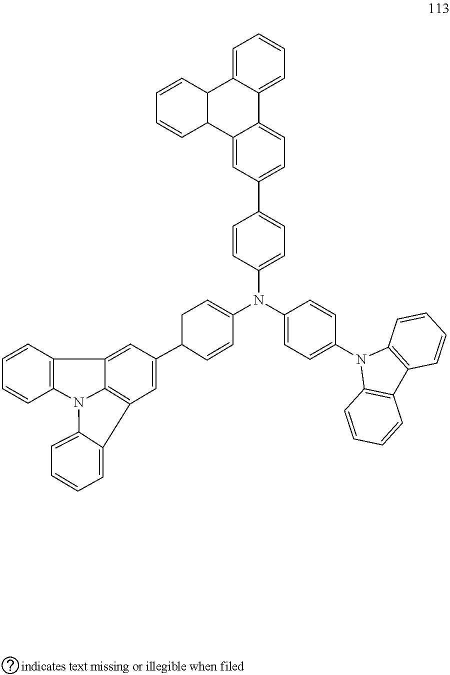 [SMILES: C1=CC2c3ccccc3-c3ccc(-c4ccc(N(C5=CCC(c6cc7c8ccccc8n8c9ccccc9c(c6)c78)C=C5)c5ccc(-n6c7ccccc7c7ccccc76)cc5)cc4)cc3C2C=C1]

In an implementation, the material for an organic EL device may include one of the following Compounds 114 to 119.

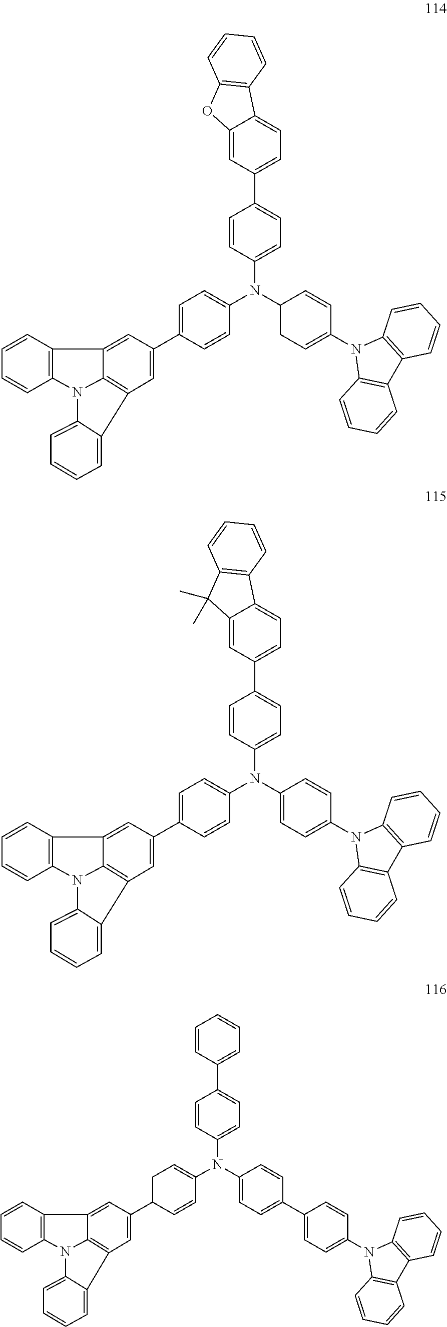 [SMILES: C1=CC(N(c2ccc(-c3ccc4c(c3)oc3ccccc34)cc2)c2ccc(-c3cc4c5ccccc5n5c6ccccc6c(c3)c45)cc2)CC=C1n1c2ccccc2c2ccccc21 ; CC1(C)c2ccccc2-c2ccc(-c3ccc(N(c4ccc(-c5cc6c7ccccc7n7c8ccccc8c(c5)c67)cc4)c4ccc(-n5c6ccccc6c6ccccc65)cc4)cc3)cc21 ; C1=CC(c2cc3c4ccccc4n4c5ccccc5c(c2)c34)CC=C1N(c1ccc(-c2ccccc2)cc1)c1ccc(-c2ccc(-n3c4ccccc4c4ccccc43)cc2)cc1]

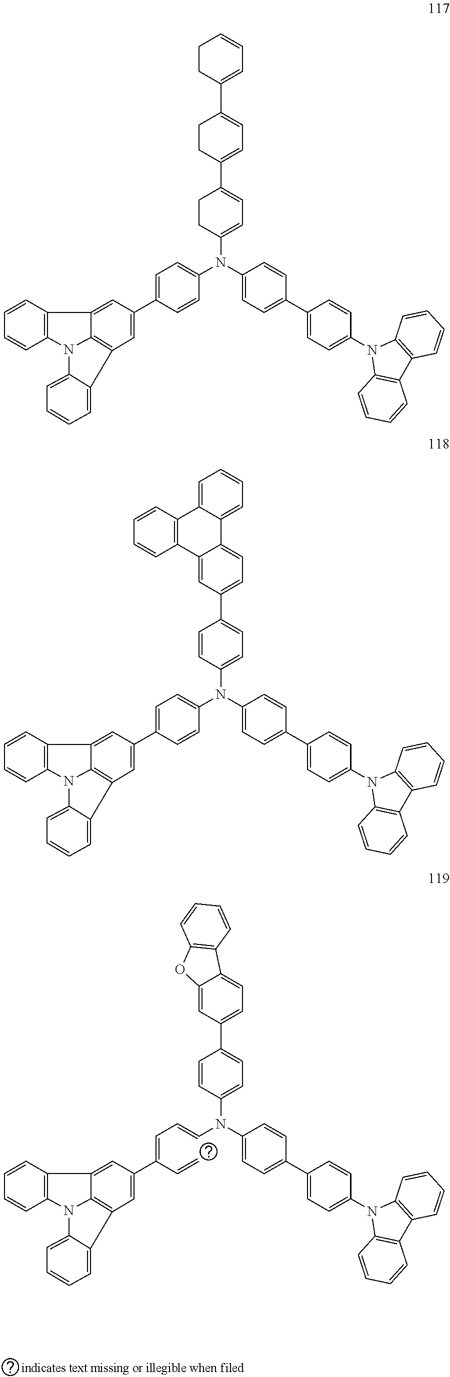 [SMILES: C1=CCCC(C2=CC=C(C3=CC=C(N(c4ccc(-c5ccc(-n6c7ccccc7c7ccccc76)cc5)cc4)c4ccc(-c5cc6c7ccccc7n7c8ccccc8c(c5)c67)cc4)CC3)CC2)=C1 ; c1ccc2c(c1)c1ccccc1c1cc(-c3ccc(N(c4ccc(-c5ccc(-n6c7ccccc7c7ccccc76)cc5)cc4)c4ccc(-c5cc6c7ccccc7n7c8ccccc8c(c5)c67)cc4)cc3)ccc21 ; C=C/C(=C\C=C\N(c1ccc(-c2ccc(-n3c4ccccc4c4ccccc43)cc2)cc1)c1ccc(-c2ccc3c(c2)oc2ccccc23)cc1)c1cc2c3ccccc3n3c4ccccc4c(c1)c23]

In an implementation, the material for an organic EL device may include one of the following Compounds 120 to 125.

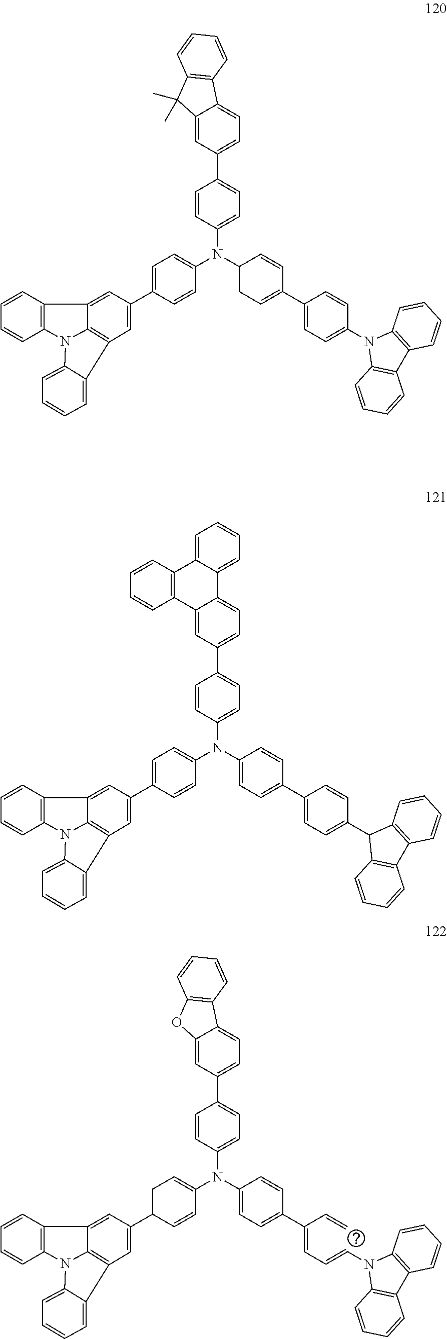 [SMILES: CC1(C)c2ccccc2-c2ccc(-c3ccc(N(c4ccc(-c5cc6c7ccccc7n7c8ccccc8c(c5)c67)cc4)C4C=CC(c5ccc(-n6c7ccccc7c7ccccc76)cc5)=CC4)cc3)cc21 ; c1ccc2c(c1)-c1ccccc1C2c1ccc(-c2ccc(N(c3ccc(-c4ccc5c6ccccc6c6ccccc6c5c4)cc3)c3ccc(-c4cc5c6ccccc6n6c7ccccc7c(c4)c56)cc3)cc2)cc1 ; C=C/C(=C\C=C\n1c2ccccc2c2ccccc21)c1ccc(N(C2=CCC(c3cc4c5ccccc5n5c6ccccc6c(c3)c45)C=C2)c2ccc(-c3ccc4c(c3)oc3ccccc34)cc2)cc1]

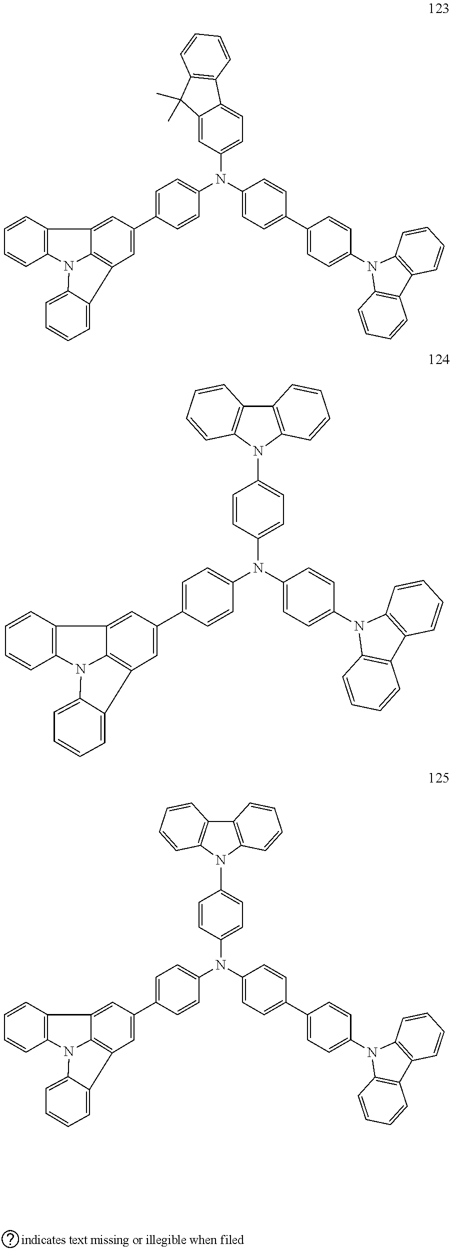 [SMILES: CC1(C)c2ccccc2-c2ccc(N(c3ccc(-c4ccc(-n5c6ccccc6c6ccccc65)cc4)cc3)c3ccc(-c4cc5c6ccccc6n6c7ccccc7c(c4)c56)cc3)cc21 ; c1ccc2c(c1)c1ccccc1n2-c1ccc(N(c2ccc(-c3cc4c5ccccc5n5c6ccccc6c(c3)c45)cc2)c2ccc(-n3c4ccccc4c4ccccc43)cc2)cc1 ; c1ccc2c(c1)c1ccccc1n2-c1ccc(-c2ccc(N(c3ccc(-c4cc5c6ccccc6n6c7ccccc7c(c4)c56)cc3)c3ccc(-n4c5ccccc5c5ccccc54)cc3)cc2)cc1]

In an implementation, the material for an organic EL device may include one of the following Compounds 126 to 134.

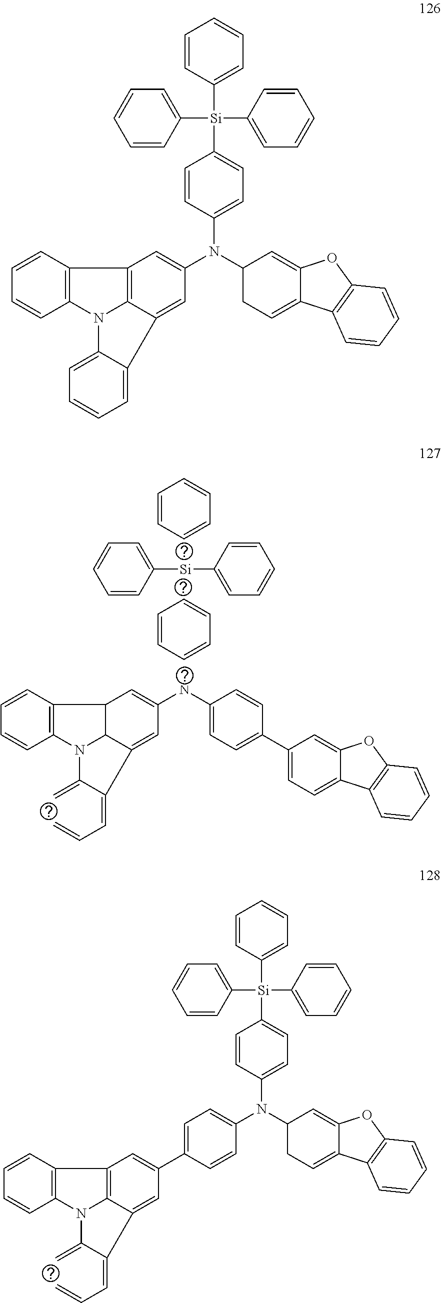

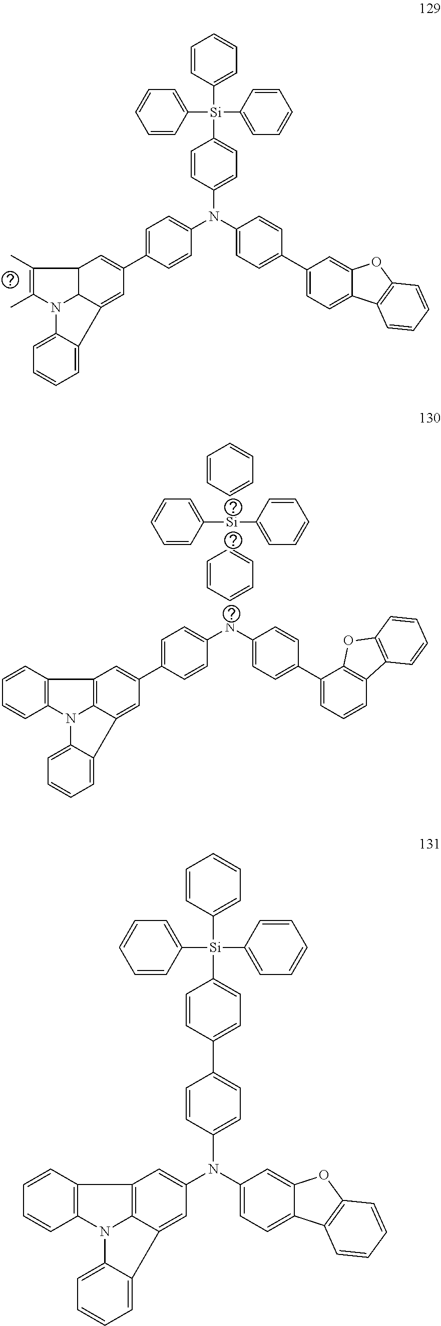

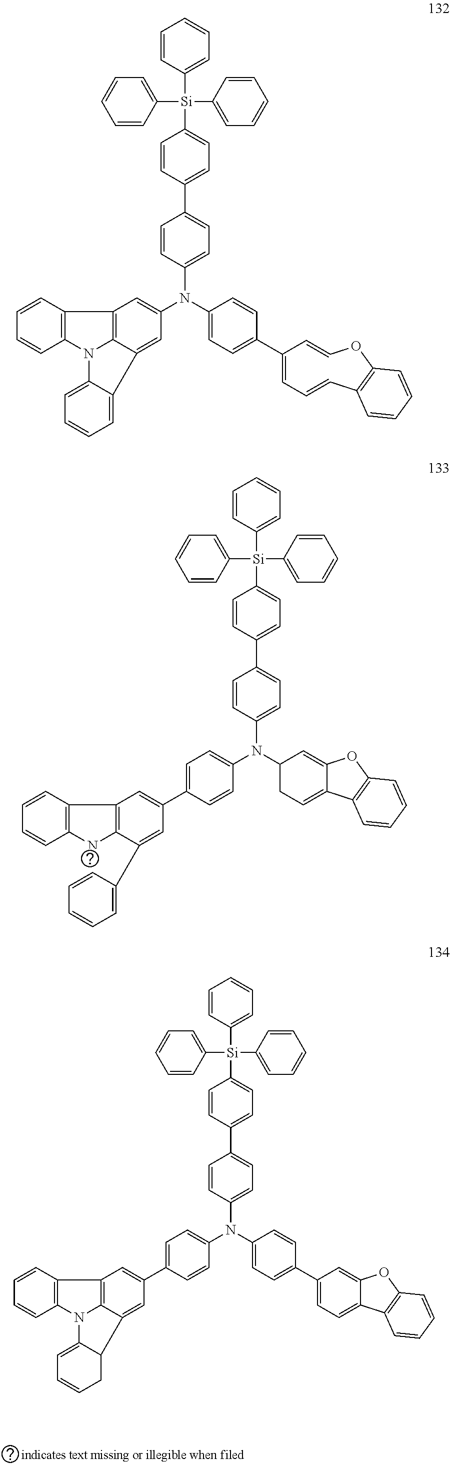 [SMILES: C1=C\c2ccccc2O/C=C/C(c2ccc(N(c3ccc(-c4ccc([Si](c5ccccc5)(c5ccccc5)c5ccccc5)cc4)cc3)c3cc4c5ccccc5n5c6ccccc6c(c3)c45)cc2)=C\1 ; C1=c2oc3ccccc3c2=CCC1N(c1ccc(-c2ccc([Si](c3ccccc3)(c3ccccc3)c3ccccc3)cc2)cc1)c1ccc(-c2cc(-c3ccccc3)c3[nH]c4ccccc4c3c2)cc1 ; C1=CCC2C(=C1)n1c3ccccc3c3cc(-c4ccc(N(c5ccc(-c6ccc([Si](c7ccccc7)(c7ccccc7)c7ccccc7)cc6)cc5)c5ccc(-c6ccc7c(c6)oc6ccccc67)cc5)cc4)cc2c31]

In an implementation, the material for an organic EL device may include one of the following Compounds 135 to 141.

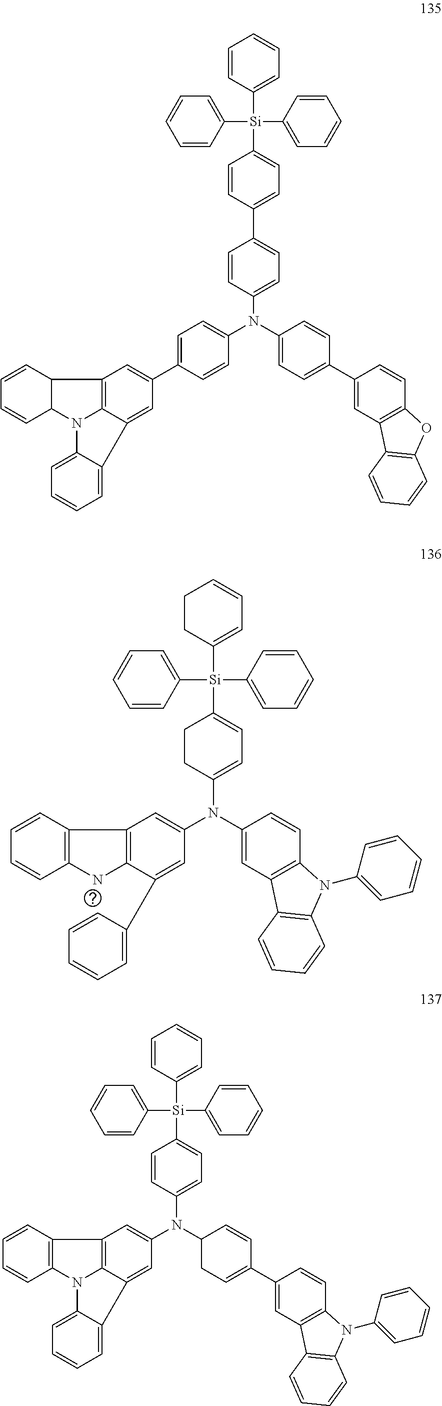 [SMILES: C1=CC2c3cc(-c4ccc(N(c5ccc(-c6ccc([Si](c7ccccc7)(c7ccccc7)c7ccccc7)cc6)cc5)c5ccc(-c6ccc7oc8ccccc8c7c6)cc5)cc4)cc4c5ccccc5n(c34)C2C=C1 ; C1=CCCC([Si](C2=CC=C(N(c3cc(-c4ccccc4)c4[nH]c5ccccc5c4c3)c3ccc4c(c3)c3ccccc3n4-c3ccccc3)CC2)(c2ccccc2)c2ccccc2)=C1 ; C1=CC(N(c2ccc([Si](c3ccccc3)(c3ccccc3)c3ccccc3)cc2)c2cc3c4ccccc4n4c5ccccc5c(c2)c34)CC=C1c1ccc2c(c1)c1ccccc1n2-c1ccccc1]

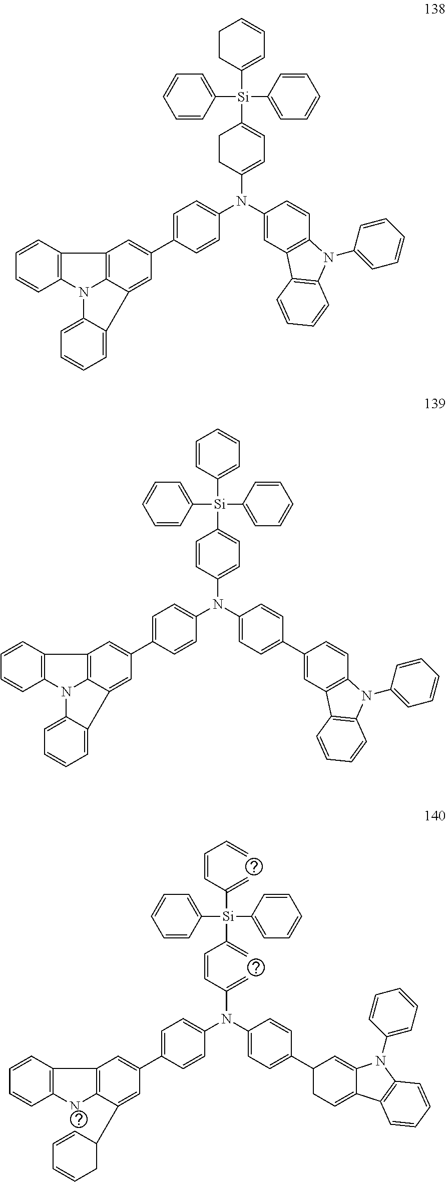 [SMILES: C1=CCCC([Si](C2=CC=C(N(c3ccc(-c4cc5c6ccccc6n6c7ccccc7c(c4)c56)cc3)c3ccc4c(c3)c3ccccc3n4-c3ccccc3)CC2)(c2ccccc2)c2ccccc2)=C1 ; c1ccc(-n2c3ccccc3c3cc(-c4ccc(N(c5ccc(-c6cc7c8ccccc8n8c9ccccc9c(c6)c78)cc5)c5ccc([Si](c6ccccc6)(c6ccccc6)c6ccccc6)cc5)cc4)ccc32)cc1 ; C=C/C=C\C(=C)[Si](C(=C)/C=C\C(=C)N(c1ccc(-c2cc(C3C=CC=CC3)c3[nH]c4ccccc4c3c2)cc1)c1ccc(C2C=c3c(c4ccccc4n3-c3ccccc3)=CC2)cc1)(c1ccccc1)c1ccccc1]

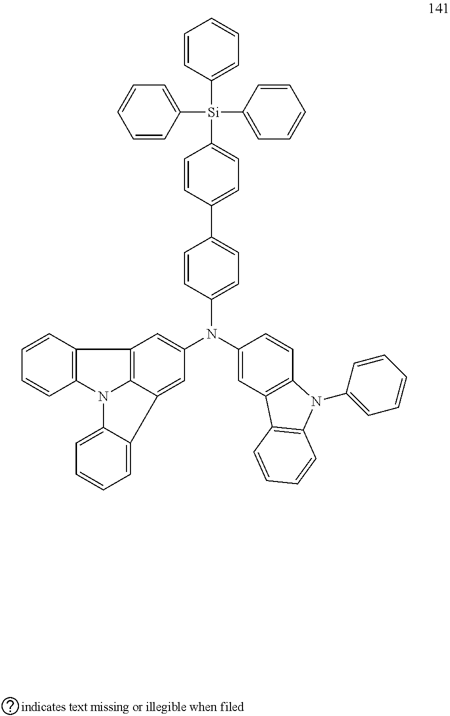 [SMILES: c1ccc(-n2c3ccccc3c3cc(N(c4ccc(-c5ccc([Si](c6ccccc6)(c6ccccc6)c6ccccc6)cc5)cc4)c4cc5c6ccccc6n6c7ccccc7c(c4)c56)ccc32)cc1]

In an implementation, the material for an organic EL device may include one of the following Compounds 142 to 148.

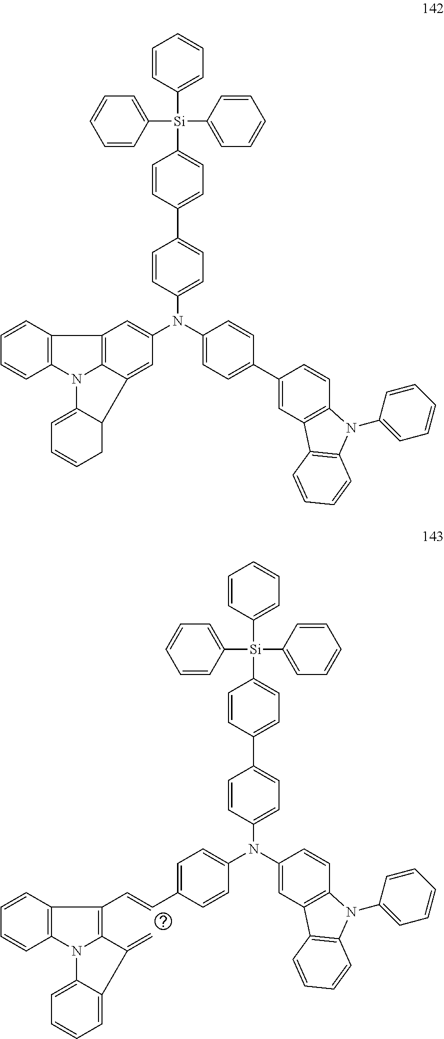 [SMILES: C1=CCC2C(=C1)n1c3ccccc3c3cc(N(c4ccc(-c5ccc([Si](c6ccccc6)(c6ccccc6)c6ccccc6)cc5)cc4)c4ccc(-c5ccc6c(c5)c5ccccc5n6-c5ccccc5)cc4)cc2c31 ; C=c1c2ccccc2n2c1c(/C=C/c1ccc(N(c3ccc(-c4ccc([Si](c5ccccc5)(c5ccccc5)c5ccccc5)cc4)cc3)c3ccc4c(c3)c3ccccc3n4-c3ccccc3)cc1)c1ccccc12]

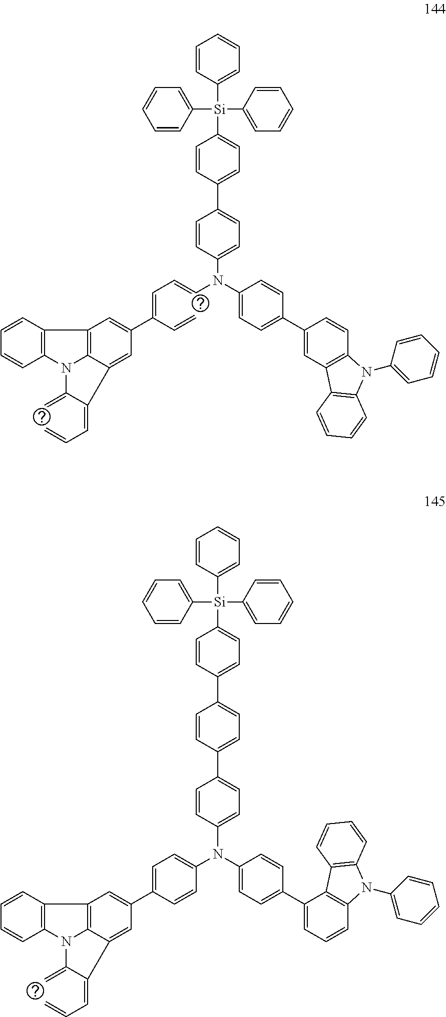 [SMILES: C=C/C=c1\c(=C)n2c3ccccc3c3cc(/C(C=C)=C/C=C/N(c4ccc(-c5ccc([Si](c6ccccc6)(c6ccccc6)c6ccccc6)cc5)cc4)c4ccc(-c5ccc6c(c5)c5ccccc5n6-c5ccccc5)cc4)cc1c32 ; C=C/C=c1\c(=C)n2c3ccccc3c3cc(-c4ccc(N(c5ccc(-c6ccc(-c7ccc([Si](c8ccccc8)(c8ccccc8)c8ccccc8)cc7)cc6)cc5)c5ccc(-c6cccc7c6c6ccccc6n7-c6ccccc6)cc5)cc4)cc1c32]

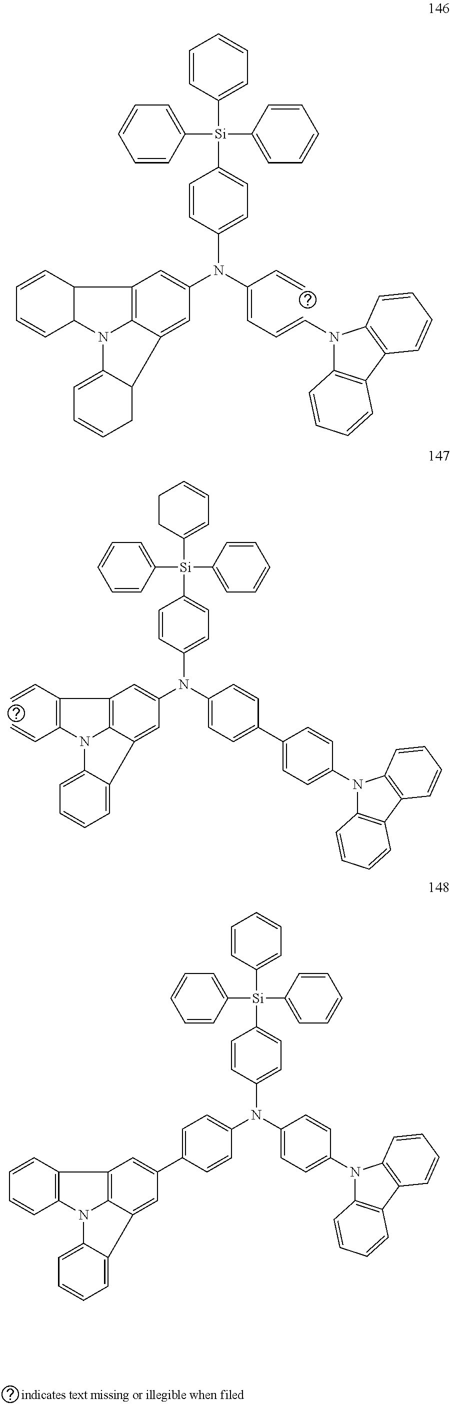 [SMILES: C=C/C(=C\C=C\n1c2ccccc2c2ccccc21)N(c1ccc([Si](c2ccccc2)(c2ccccc2)c2ccccc2)cc1)c1cc2c3c(c1)C1C=CC=CC1N3C1=CC=CCC12 ; C=Cc1c(C=C)n2c3ccccc3c3cc(N(c4ccc(-c5ccc(-n6c7ccccc7c7ccccc76)cc5)cc4)c4ccc([Si](C5=CC=CCC5)(c5ccccc5)c5ccccc5)cc4)cc1c32 ; c1ccc([Si](c2ccccc2)(c2ccccc2)c2ccc(N(c3ccc(-c4cc5c6ccccc6n6c7ccccc7c(c4)c56)cc3)c3ccc(-n4c5ccccc5c5ccccc54)cc3)cc2)cc1]

In an implementation, the material for an organic EL device may include one of the following Compounds 149 to 154.

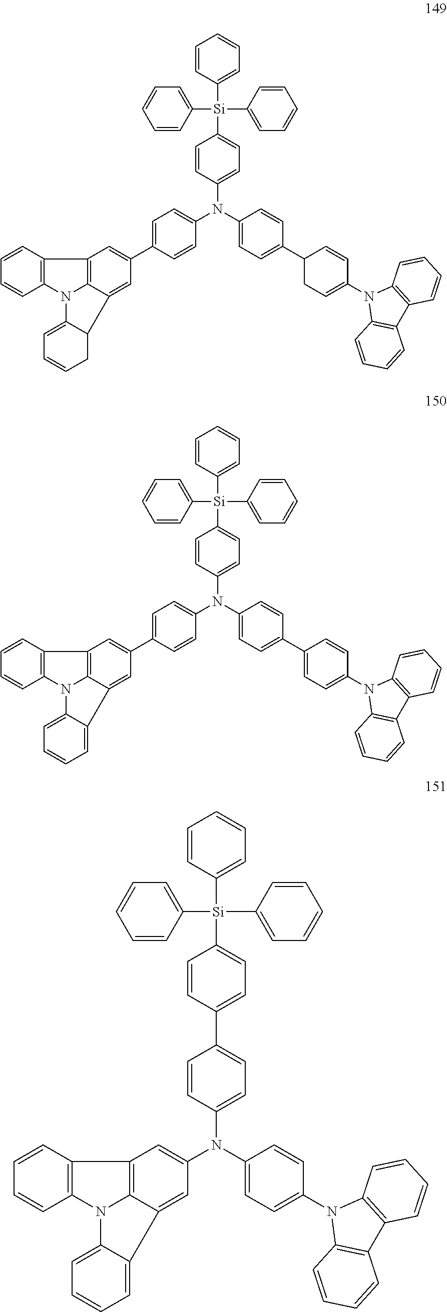 [SMILES: C1=CCC2C(=C1)n1c3ccccc3c3cc(-c4ccc(N(c5ccc(C6C=CC(n7c8ccccc8c8ccccc87)=CC6)cc5)c5ccc([Si](c6ccccc6)(c6ccccc6)c6ccccc6)cc5)cc4)cc2c31 ; c1ccc([Si](c2ccccc2)(c2ccccc2)c2ccc(N(c3ccc(-c4ccc(-n5c6ccccc6c6ccccc65)cc4)cc3)c3ccc(-c4cc5c6ccccc6n6c7ccccc7c(c4)c56)cc3)cc2)cc1 ; c1ccc([Si](c2ccccc2)(c2ccccc2)c2ccc(-c3ccc(N(c4ccc(-n5c6ccccc6c6ccccc65)cc4)c4cc5c6ccccc6n6c7ccccc7c(c4)c56)cc3)cc2)cc1]

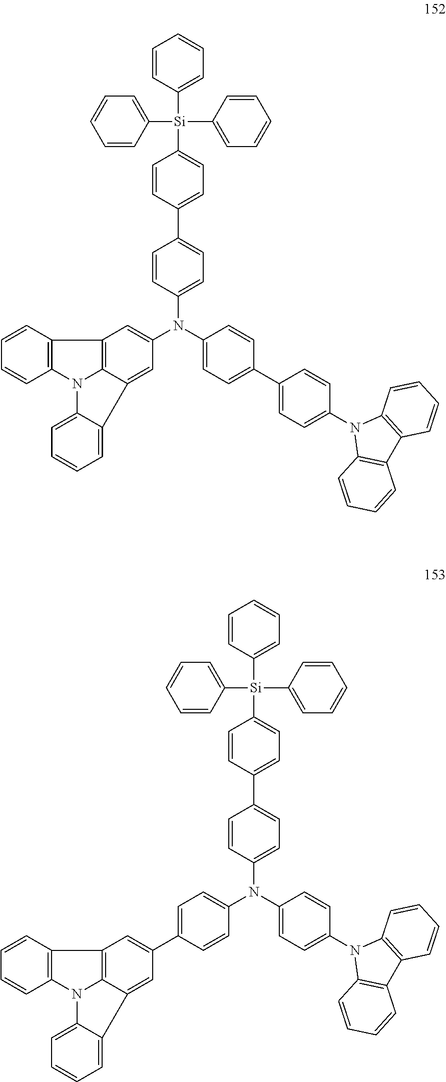 [SMILES: c1ccc([Si](c2ccccc2)(c2ccccc2)c2ccc(-c3ccc(N(c4ccc(-c5ccc(-n6c7ccccc7c7ccccc76)cc5)cc4)c4cc5c6ccccc6n6c7ccccc7c(c4)c56)cc3)cc2)cc1 ; c1ccc([Si](c2ccccc2)(c2ccccc2)c2ccc(-c3ccc(N(c4ccc(-c5cc6c7ccccc7n7c8ccccc8c(c5)c67)cc4)c4ccc(-n5c6ccccc6c6ccccc65)cc4)cc3)cc2)cc1]

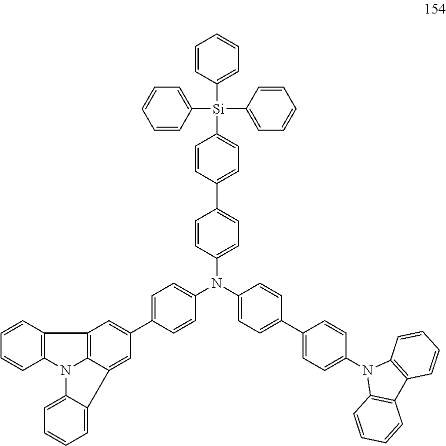 [SMILES: c1ccc([Si](c2ccccc2)(c2ccccc2)c2ccc(-c3ccc(N(c4ccc(-c5ccc(-n6c7ccccc7c7ccccc76)cc5)cc4)c4ccc(-c5cc6c7ccccc7n7c8ccccc8c(c5)c67)cc4)cc3)cc2)cc1]

In an implementation, the material for an organic EL device may include one of the following Compounds 155 to 160.

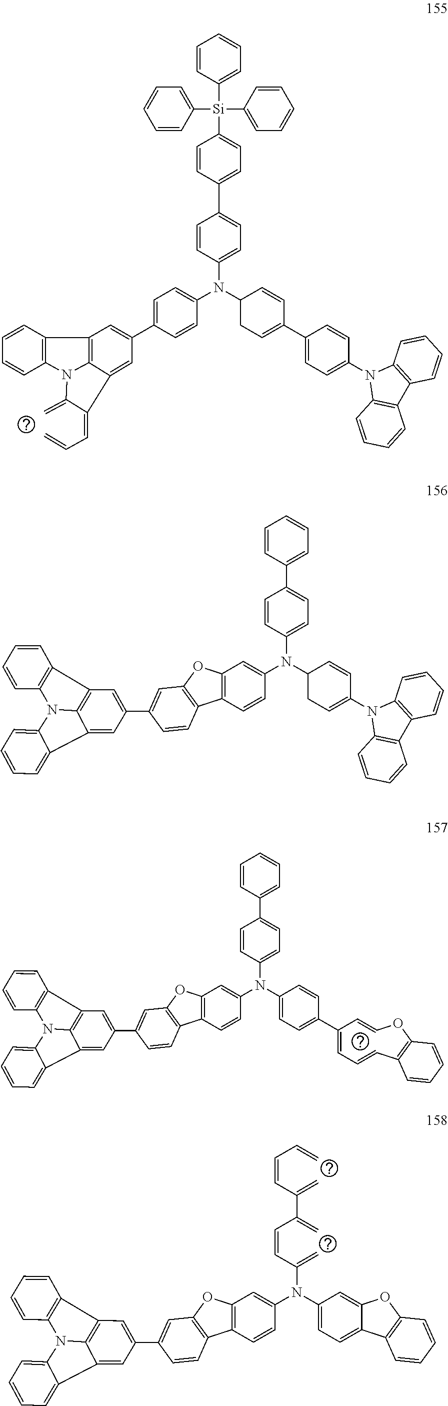 [SMILES: C=C/C=c1\c(=C)n2c3ccccc3c3cc(-c4ccc(N(c5ccc(-c6ccc([Si](c7ccccc7)(c7ccccc7)c7ccccc7)cc6)cc5)C5C=CC(c6ccc(-n7c8ccccc8c8ccccc87)cc6)=CC5)cc4)cc1c32 ; C1=CC(N(c2ccc(-c3ccccc3)cc2)c2ccc3c(c2)oc2cc(-c4cc5c6ccccc6n6c7ccccc7c(c4)c56)ccc23)CC=C1n1c2ccccc2c2ccccc21 ; C1=C\c2ccccc2O/C=C/C(c2ccc(N(c3ccc(-c4ccccc4)cc3)c3ccc4c(c3)oc3cc(-c5cc6c7ccccc7n7c8ccccc8c(c5)c67)ccc34)cc2)=C\1 ; C=C/C=C\C(=C)C(=C)/C=C\C(=C)N(c1ccc2c(c1)oc1ccccc12)c1ccc2c(c1)oc1cc(-c3cc4c5ccccc5n5c6ccccc6c(c3)c45)ccc12]

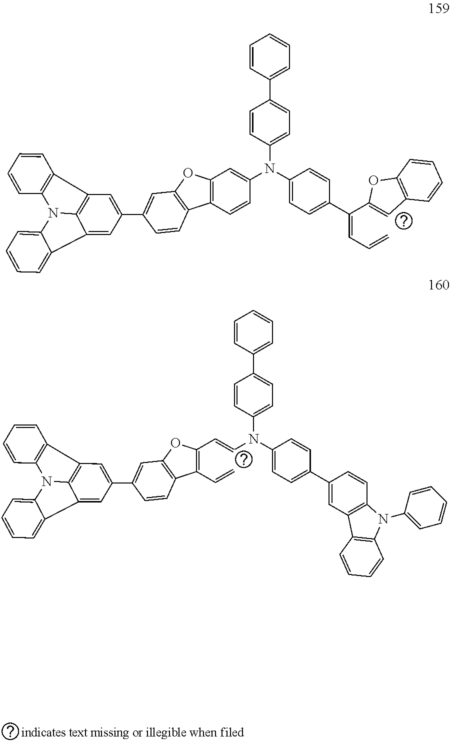 [SMILES: C=C/C=C(/c1ccc(N(c2ccc(-c3ccccc3)cc2)c2ccc3c(c2)oc2cc(-c4cc5c6ccccc6n6c7ccccc7c(c4)c56)ccc23)cc1)c1cc2ccccc2o1 ; C=Cc1c(/C=C/N(c2ccc(-c3ccccc3)cc2)c2ccc(-c3ccc4c(c3)c3ccccc3n4-c3ccccc3)cc2)oc2cc(-c3cc4c5ccccc5n5c6ccccc6c(c3)c45)ccc12]

In an implementation, the material for an organic EL device may include one of the following Compounds 161 to 167.

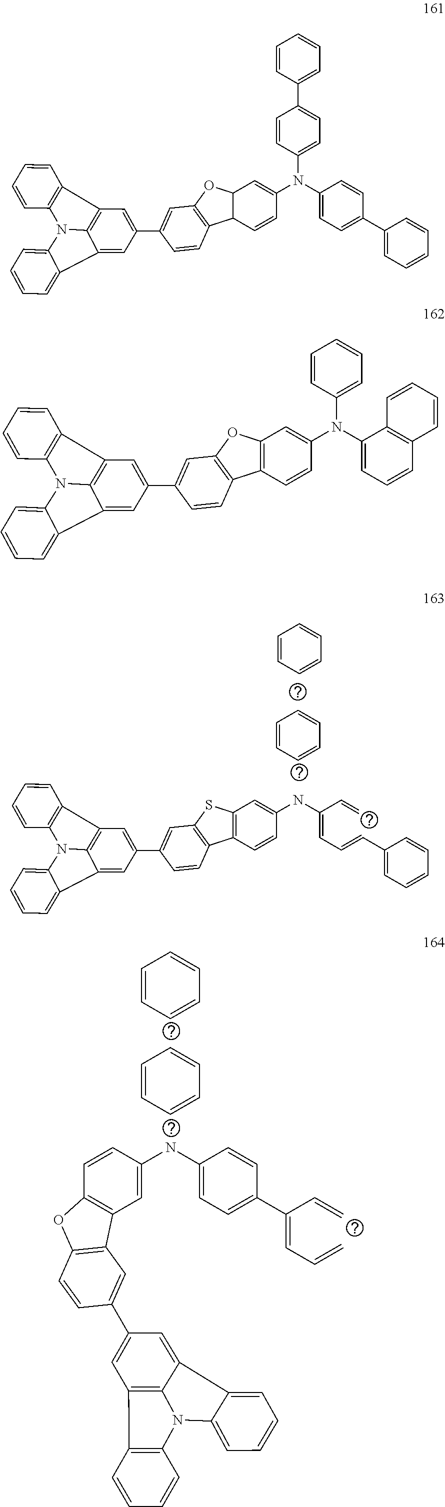

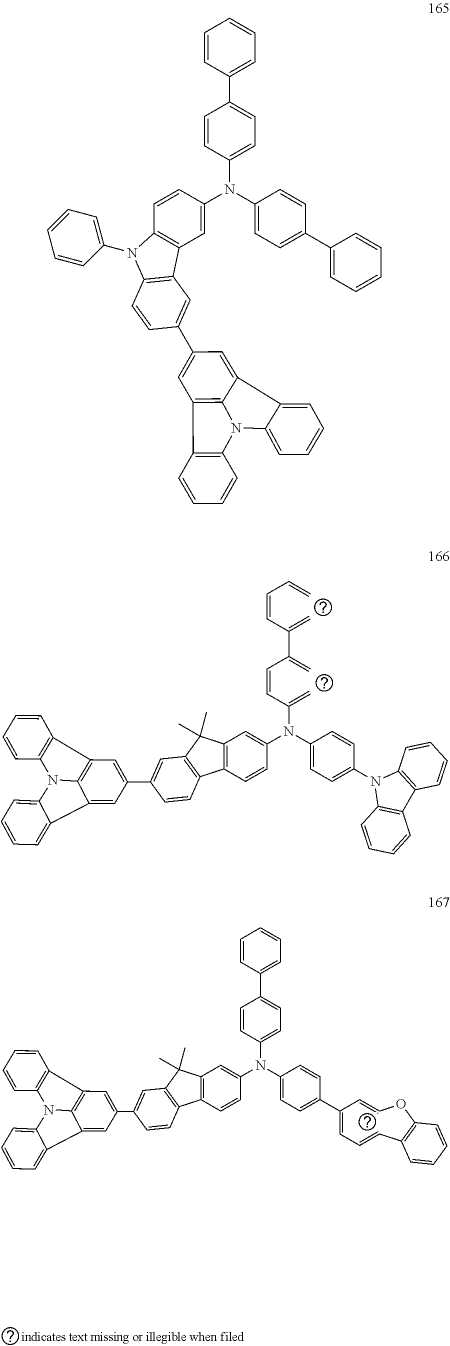 [SMILES: c1ccc(-c2ccc(N(c3ccc(-c4ccccc4)cc3)c3ccc4c(c3)c3cc(-c5cc6c7ccccc7n7c8ccccc8c(c5)c67)ccc3n4-c3ccccc3)cc2)cc1 ; C=C/C=C\C(=C)C(=C)/C=C\C(=C)N(c1ccc(-n2c3ccccc3c3ccccc32)cc1)c1ccc2c(c1)C(C)(C)c1cc(-c3cc4c5ccccc5n5c6ccccc6c(c3)c45)ccc1-2 ; CC1(C)c2cc(-c3cc4c5ccccc5n5c6ccccc6c(c3)c45)ccc2-c2ccc(N(c3ccc(C4=C/C=C/c5ccccc5O\C=C\4)cc3)c3ccc(-c4ccccc4)cc3)cc21]

In an implementation, the material for an organic EL device may include one of the following Compounds 168 to 170.

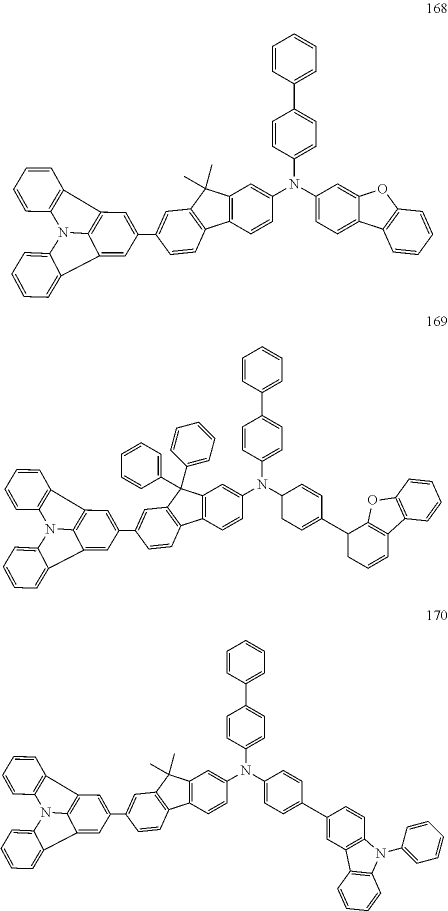 [SMILES: CC1(C)c2cc(-c3cc4c5ccccc5n5c6ccccc6c(c3)c45)ccc2-c2ccc(N(c3ccc(-c4ccccc4)cc3)c3ccc4c(c3)oc3ccccc34)cc21 ; C1=Cc2c(oc3ccccc23)C(C2=CCC(N(c3ccc(-c4ccccc4)cc3)c3ccc4c(c3)C(c3ccccc3)(c3ccccc3)c3cc(-c5cc6c7ccccc7n7c8ccccc8c(c5)c67)ccc3-4)C=C2)C1 ; CC1(C)c2cc(-c3cc4c5ccccc5n5c6ccccc6c(c3)c45)ccc2-c2ccc(N(c3ccc(-c4ccccc4)cc3)c3ccc(-c4ccc5c(c4)c4ccccc4n5-c4ccccc4)cc3)cc21]

The material for an organic EL device according to an embodiment may be used or included in a layer (of stacking or stacked layers) between an anode and an emission layer. For example, the material may be used as a hole transport material for an organic EL device. In addition, by using the material for an organic EL device according to an embodiment for the formation of the hole transport layer, an organic EL device driven at a low voltage and having high efficiency and long life may be manufactured.

In an implementation, the material for an organic EL device according to an embodiment may be used as a material of a hole injection layer. In the case that the material for an organic EL device according to an embodiment is used as the material for the hole injection layer, deterioration of the hole injection layer due to electrons may be restrained. Thus, the long life of an organic EL device may be realized as in the case of using the material in the hole transport layer. In an implementation, the diamine derivative according to an embodiment may have electron tolerance, and the material may be used as a host material of an emission layer.

(Organic EL Device)

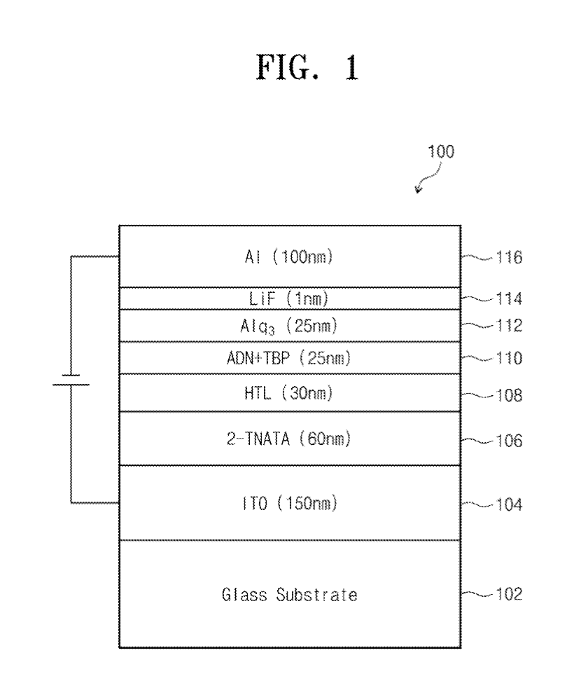

An organic EL device using or including the material for an organic EL device according to an embodiment will be explained. FIG. 1 illustrates a schematic diagram of an organic EL device 100 according to an embodiment. The organic EL device 100 may include, e.g., a substrate 102, a anode 104, a hole injection layer 106, a hole transport layer 108, an emission layer 110, an electron transport layer 112, an electron injection layer 114, and a cathode 116. In an implementation, the material for an organic EL device according to an embodiment may be used or included in the hole transport layer.

For example, an embodiment in which the material for an organic EL device is included in the hole transport layer 108 will be explained. The substrate 102 may be a transparent glass substrate, a semiconductor substrate formed by using silicon, etc., or a flexible substrate of a resin, etc. The anode 104 may be disposed on the substrate 102 and may be formed by using indium tin oxide (ITO), indium zinc oxide (IZO), etc. The hole injection layer 106 may be disposed on the anode 104 and may include, for example, 4,4′,4″-tris[2-naphthyl(phenyl)amino]triphenylamine (2-TNATA) or N,N,N′,N′-tetrakis(3-methylphenyl)-3,3′-dimethylbenzidine (HMTPD), etc. The hole transport layer 108 may be disposed on the hole injection layer 106 and may be formed by using the material for an organic EL device according to an embodiment. The emission layer 110 may be disposed on the hole transport layer 108 and may be formed by using the material for an organic EL device according to an embodiment. In an implementation, the emission layer 110 may be formed by using, e.g., a host material including 9,10-di(2-naphthyl)anthracene (ADN) doped with 2,5,8,11-tetra-t-butylperylene (TBP). The electron transport layer 112 may be disposed on the emission layer 110 and may be formed by using, e.g., a material including tris(8-hydroxyquinolinato)aluminum (Alq3). The electron injection layer 114 may be disposed on the electron transport layer 112 and may be formed by using, e.g., a material including lithium fluoride (LiF). The cathode 116 may be disposed on the electron injection layer 114 and may be formed by using a metal such as Al or a transparent material such as ITO, IZO, etc. The above-described thin layers may be formed by selecting an appropriate layer forming method such as vacuum deposition, sputtering, various coatings, etc.

In the organic EL device 100 according to an embodiment, a hole transport layer driven at a low voltage and having high efficiency and long life may be formed by using the material for an organic EL device according to an embodiment. In addition, the material for an organic EL device according to an embodiment may be applied in an organic EL apparatus of an active matrix type using thin film transistors (TFT).

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### EXAMPLES

(Preparation Method)

The above-described materials for an organic EL device may be synthesized, e.g., by the following method.

(Synthesis of Compound 16)

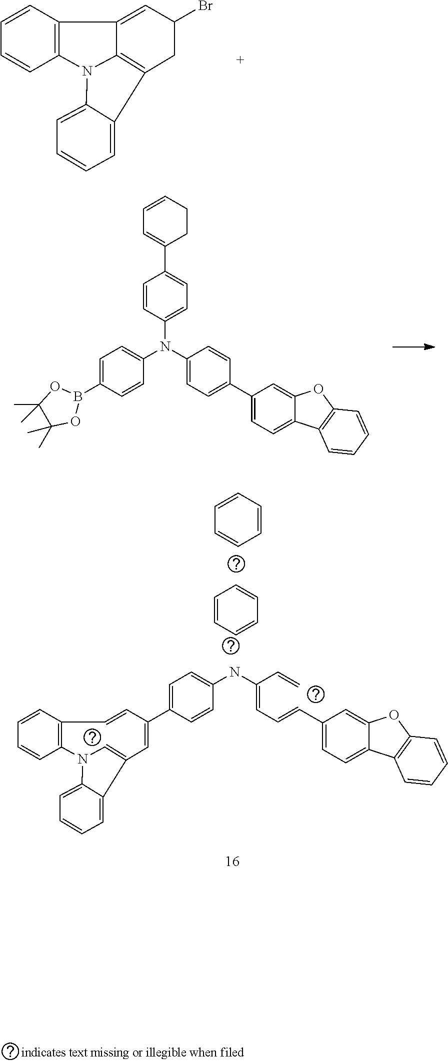 [SMILES: BrC1C=c2c3ccccc3n3c2c(c2ccccc23)C1 ; CC1(C)OB(c2ccc(N(c3ccc(C4=CC=CCC4)cc3)c3ccc(-c4ccc5c(c4)oc4ccccc45)cc3)cc2)OC1(C)C ; c1ccccc1 ; c1ccccc1 ; C=C/C(=C\C=C\c1ccc2c(c1)oc1ccccc12)Nc1ccc(-c2ccc3ccccc3n3cc(c2)c2ccccc23)cc1]

An amine compound (4 mmol), above, an indolocarbazole compound (4 mmol), above, a palladium catalyst (0.4 mol), a phosphine ligand (1.6 mol), an alkaline reagent (16 mmol), toluene (250 mL), water (25 mL) and ethanol (13 mL) were added in a reaction vessel, followed by charging nitrogen in the vessel and stirring while refluxing for 20 hours. After cooling, water was added in the reactant, and extraction of an organic layer was performed. The obtained organic layer was dried with magnesium sulfate and filtered, and the obtained filtrate was concentrated using a rotary evaporator. The obtained crude product was separated by silica gel column chromatography, and the obtained solid was recrystallized to produce Compound 16 Formula 25 with a yield of 55% (APCI+: C54H32N2O, measured value 726).

(Synthesis of Compound 116)

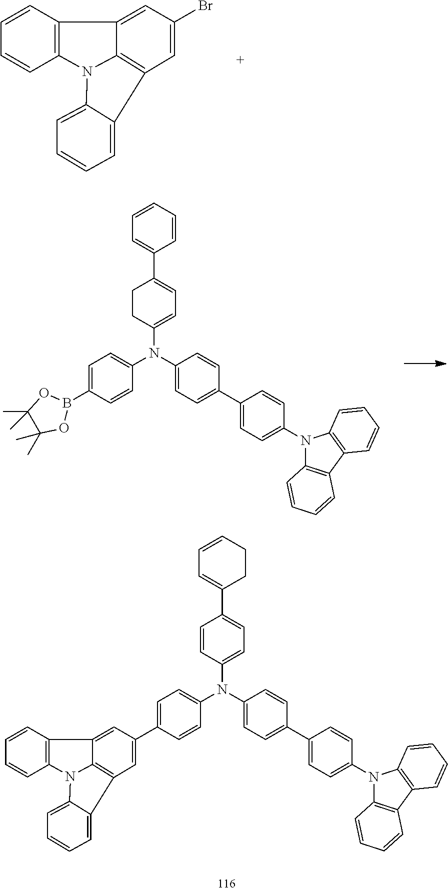 [SMILES: Brc1cc2c3ccccc3n3c4ccccc4c(c1)c23 ; CC1(C)OB(c2ccc(N(C3=CC=C(c4ccccc4)CC3)c3ccc(-c4ccc(-n5c6ccccc6c6ccccc65)cc4)cc3)cc2)OC1(C)C ; C1=CCCC(c2ccc(N(c3ccc(-c4ccc(-n5c6ccccc6c6ccccc65)cc4)cc3)c3ccc(-c4cc5c6ccccc6n6c7ccccc7c(c4)c56)cc3)cc2)=C1]

An amine compound (6 mmol), above, an indolocarbazole compound (6 mmol), above, a palladium catalyst (0.6 mol), a phosphine ligand (2.4 mol), an alkaline reagent (24 mmol), toluene (350 mL), water (35 mL) and ethanol (18 mL) were added in a reaction vessel, followed by charging nitrogen in the vessel and stirring while refluxing for 18 hours. After cooling, water was added in the reactant, and extraction of an organic layer was performed. The obtained organic layer was dried with magnesium sulfate and filtered, and the obtained filtrate was concentrated using a rotary evaporator. The obtained crude product was separated by silica gel column chromatography, and the obtained solid was recrystallized to produce Compound 116 with a yield of 47% (APCI+: C60H39N3, measured value 801).

(Synthesis of Compound 128)

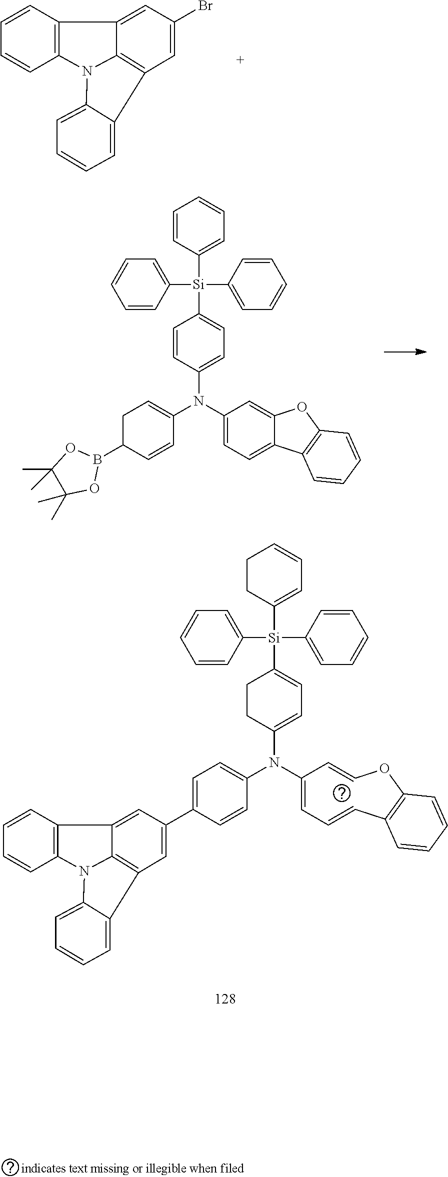 [SMILES: Brc1cc2c3ccccc3n3c4ccccc4c(c1)c23 ; CC1(C)OB(C2C=CC(N(c3ccc([Si](c4ccccc4)(c4ccccc4)c4ccccc4)cc3)c3ccc4c(c3)oc3ccccc34)=CC2)OC1(C)C ; C1=CCCC([Si](C2=CC=C(N(C3=C/C=C/c4ccccc4O\C=C\3)c3ccc(-c4cc5c6ccccc6n6c7ccccc7c(c4)c56)cc3)CC2)(c2ccccc2)c2ccccc2)=C1]

An amine compound (5.5 mmol), above, an indolocarbazole compound (5.5 mmol), above, a palladium catalyst (0.6 mol), a phosphine ligand (2.4 mol), an alkaline reagent (22 mmol), toluene (300 mL), water (30 mL) and ethanol (15 mL) were added in a reaction vessel, followed by charging nitrogen in the vessel and stirring while refluxing for 18 hours. After cooling, water was added in the reactant, and extraction of an organic layer was performed. The obtained organic layer was dried with magnesium sulfate and filtered, and the obtained filtrate was concentrated using a rotary evaporator. The obtained crude product was separated by silica gel column chromatography, and the obtained solid was recrystallized to produce Compound 128 with a yield of 50% (APCI+: C60H4ON2OSi, measured value 832).

(Synthesis of Compound 162)

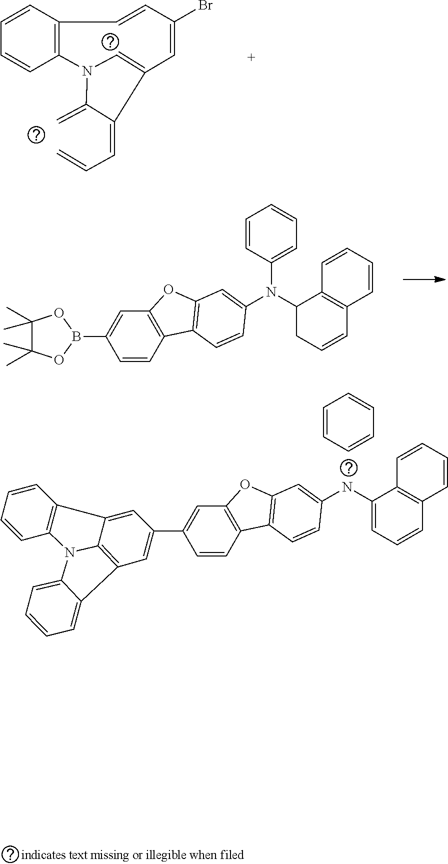

An amine compound (3.5 mmol), above, an indolocarbazole compound (3.5 mmol), above, a palladium catalyst (0.4 mol), a phosphine ligand (1.6 mol), an alkaline reagent (14 mmol), toluene (300 mL), water (30 mL) and ethanol (15 mL) were added in a reaction vessel, followed by charging nitrogen in the vessel and stirring while refluxing for 19 hours. After cooling, water was added in the reactant, and extraction of an organic layer was performed. The obtained organic layer was dried with magnesium sulfate and filtered, and the obtained filtrate was concentrated using a rotary evaporator. The obtained crude product was separated by silica gel column chromatography, and the obtained solid was recrystallized to produce Compound 162 with a yield of 65% (APCI+: C46H28N2O, measured value 624).

Organic EL devices according to Examples 1 to 4 were manufactured by using the above Compounds 16, 116, 128, and 162 as hole transport materials. In addition, organic EL devices according to Comparative Examples 1 and 2 were manufactured by using the following Comparative Compounds 1 and 2 as hole transport materials, for comparison.

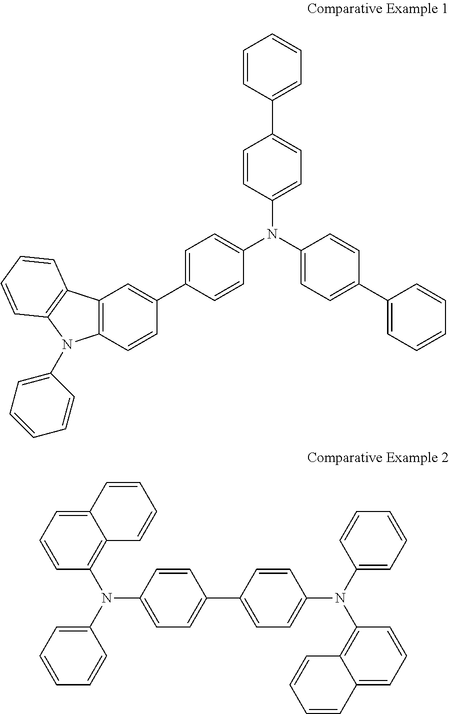 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(N(c2ccc(-c3ccc(N(c4ccccc4)c4cccc5ccccc45)cc3)cc2)c2cccc3ccccc23)cc1]

The substrate 102 was formed using a transparent glass substrate, the anode 104 was formed using ITO to a thickness of about 150 nm, the hole injection layer 106 was formed using TNATA to a thickness of about 60 nm, the hole transport layer 108 was formed using the compounds according to the Examples and the Comparative Examples to a thickness of about 30 nm, the emission layer 110 was formed using ADN doped with 3% TBP to a thickness of about 25 nm, the electron transport layer 112 was formed using Alq3 to a thickness of about 25 nm, the electron injection layer 114 was formed using LiF to a thickness of about 1 nm, and the cathode 116 was formed using Al to a thickness of about 100 nm.

With respect to the organic EL devices thus manufactured, the voltage, the emission efficiency and the life were evaluated. The values were measured and evaluated at current density of 10 mA/cm² and half life of 1,000 cd/m².

TABLE 1

| | Hole transport | Voltage | Current efficiency | |
|---|---|---|---|---|
| | material | (V) | (cd/A) | Life (hr) |
| Example 1 | Compound 6 | 6.5 | 6.9 | 2,800 |
| Example 2 | Compound 116 | 6.3 | 7.3 | 2,500 |
| Example 3 | Compound 128 | 6.9 | 7.7 | 2,300 |
| Example 4 | Compound 162 | 6.8 | 7.6 | 2,400 |
| Comparative | Comparative | 7.5 | 6.2 | 1,500 |
| Example 1 | Compound 1 | | | |
| Comparative | Comparative | 8.1 | 5.3 | 1,200 |
| Example 2 | Compound 2 | | | |

As may be seen in Table 1, organic EL devices including the amine compound combined at the position 2 of an indolo[3,2,1-jk] carbazolyl group in the hole transport layer were driven at a lower voltage and had increased emission efficiency and increased half life, when compared to the organic EL device of Comparative Example 1 (including an amine compound having a carbazolyl group in the hole transport layer) and an organic EL device of Comparative Example 2 (including a diamine compound combined with an aryl group in the hole transport layer).

By way of summation and review, in the application of the organic EL device in a display apparatus, driving at a low voltage, high efficiency, and long life of the organic EL device may be desirable. The normalization, the stabilization, and the durability of a hole transport layer or an emission layer may be considered to help realize the high efficiency and the long life of the organic EL device. A material used in a hole transport layer may include various compounds such as an aromatic amine-based compound. For example, a carbazole derivative may be used as a hole transport material or a hole injection material. In addition, an amine compound having a terphenyl group may be as a hole transport material and a host material in an emission layer. An amine compound having a fluorenyl group may be as a hole transport material or a hole injection material. A diarylamine compound combined with an indolocarbazolyl group at a para position thereof via at least one phenyl group may be as a hole injection material, a hole transport material, or an electron inhibiting material.

Organic EL devices using such materials may not have sufficient emission efficiency and emission life. A material for an organic EL device having higher efficiency and longer emission life may be desirable.

The embodiments may provide a material for an organic electroluminescence device that is driven at a low voltage, and that has high efficiency and long life in, e.g., a blue emission region and a green emission region.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A material for an organic electroluminescence (EL) device, the material being represented by the following Formula 1: 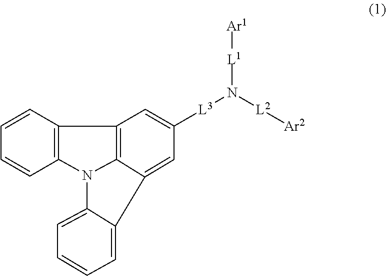
wherein, in Formula 1,
Ar¹ and Ar² are each independently a substituted or unsubstituted aryl group or a substituted or unsubstituted heteroaryl group,
L¹, L², and L³ are each independently a single bond, a substituted or unsubstituted arylene group, or a substituted or unsubstituted heteroarylene group, and
at least one of Ar¹, Ar², L¹, L², and L³ is a substituted or unsubstituted heteroaryl or heteroarylene group.

2. The material for an organic EL device as claimed in claim 1, wherein:
at least one of Ar¹ and Ar² is a substituted or unsubstituted heteroaryl group, and
L¹, L², and L³ are each independently a single bond or a substituted or unsubstituted arylene group having 6 to 18 ring carbon atoms.

3. The material for an organic EL device as claimed in claim 1, wherein Ar¹ and Ar² are each independently a substituted or unsubstituted aryl group having 6 to 24 ring carbon atoms for forming a ring, a substituted or unsubstituted carbazolyl group, a substituted or unsubstituted dibenzofuryl group, or a substituted or unsubstituted dibenzothienyl group.

4. An organic electroluminescence (EL) device comprising a material represented by the following Formula 1: 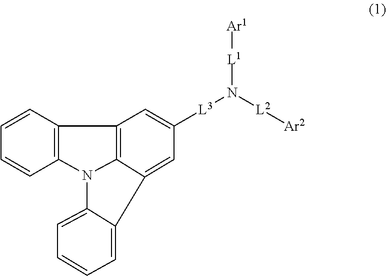
wherein, in Formula 1,
Ar¹ and Ar² are each independently a substituted or unsubstituted aryl group or a substituted or unsubstituted heteroaryl group,
L¹, L², and L³ are each independently a single bond, a substituted or unsubstituted arylene group, or a substituted or unsubstituted heteroarylene group, and
at least one of Ar¹, Ar², L¹, L², and L³ is a substituted or unsubstituted heteroaryl or heteroarylene group.

5. The organic EL device as claimed in claim 4, wherein the material is a hole transport material.

6. The organic EL device as claimed in claim 4, wherein:
the organic EL device includes an emission layer and an anode, and
the material is included in a layer that is between the emission layer and the anode.

7. The organic EL device as claimed in claim 4, wherein the material is one of the following Compounds 1 to 18:

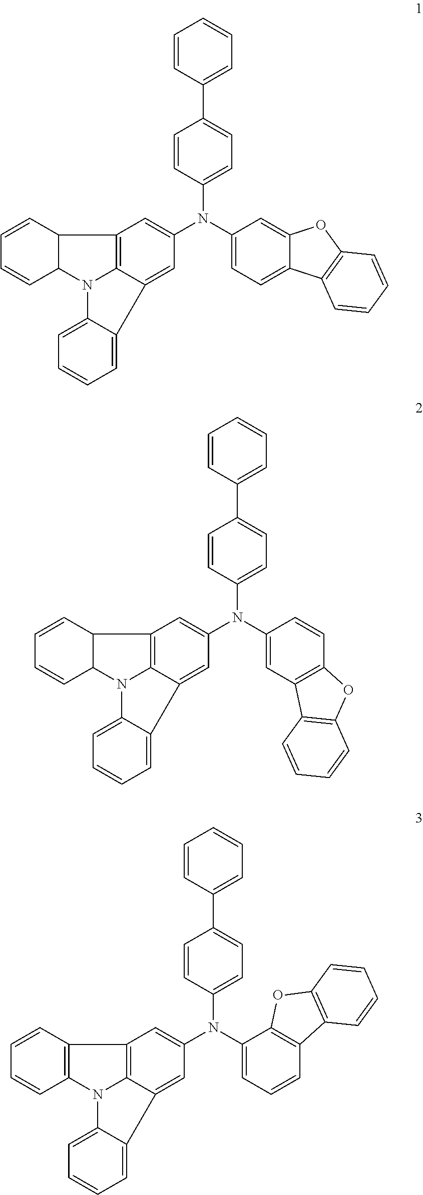 [SMILES: C1=CC2c3cc(N(c4ccc(-c5ccccc5)cc4)c4ccc5c(c4)oc4ccccc45)cc4c5ccccc5n(c34)C2C=C1 ; C1=CC2c3cc(N(c4ccc(-c5ccccc5)cc4)c4ccc5oc6ccccc6c5c4)cc4c5ccccc5n(c34)C2C=C1 ; c1ccc(-c2ccc(N(c3cc4c5ccccc5n5c6ccccc6c(c3)c45)c3cccc4c3oc3ccccc34)cc2)cc1]

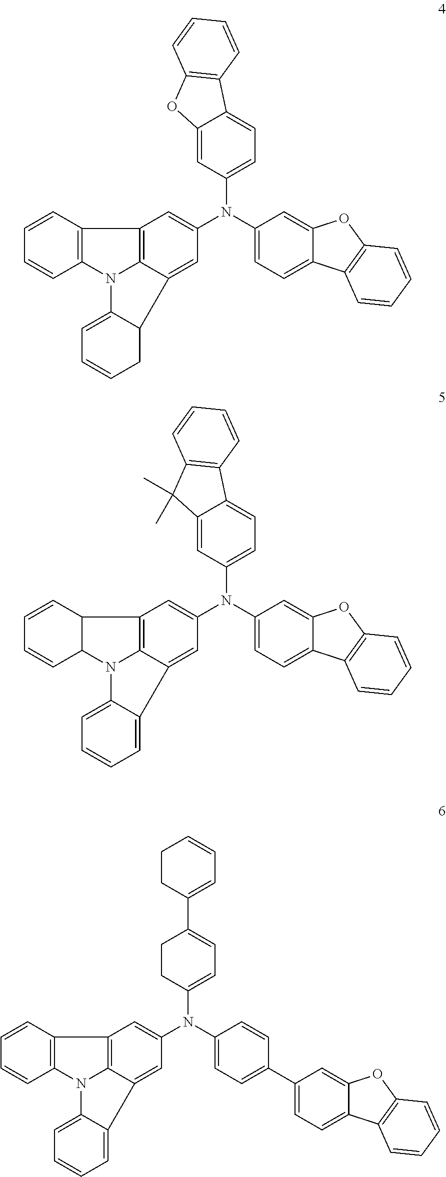 [SMILES: C1=CCC2C(=C1)n1c3ccccc3c3cc(N(c4ccc5c(c4)oc4ccccc45)c4ccc5c(c4)oc4ccccc45)cc2c31 ; CC1(C)c2ccccc2-c2ccc(N(c3ccc4c(c3)oc3ccccc34)c3cc4c5c(c3)c3ccccc3n5C3C=CC=CC43)cc21 ; C1=CCCC(C2=CC=C(N(c3ccc(-c4ccc5c(c4)oc4ccccc45)cc3)c3cc4c5ccccc5n5c6ccccc6c(c3)c45)CC2)=C1]

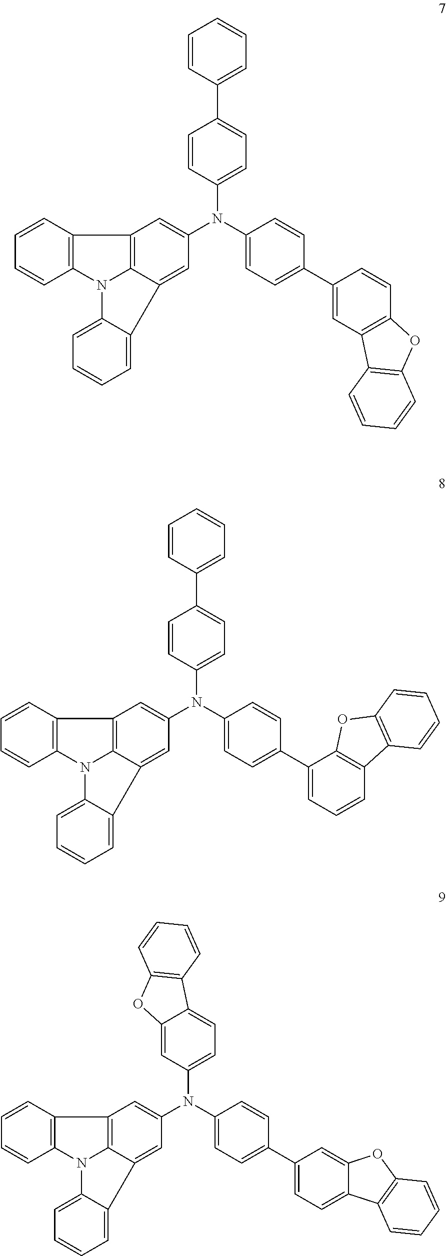 [SMILES: c1ccc(-c2ccc(N(c3ccc(-c4ccc5oc6ccccc6c5c4)cc3)c3cc4c5ccccc5n5c6ccccc6c(c3)c45)cc2)cc1 ; c1ccc(-c2ccc(N(c3ccc(-c4cccc5c4oc4ccccc45)cc3)c3cc4c5ccccc5n5c6ccccc6c(c3)c45)cc2)cc1 ; c1ccc2c(c1)oc1cc(-c3ccc(N(c4ccc5c(c4)oc4ccccc45)c4cc5c6ccccc6n6c7ccccc7c(c4)c56)cc3)ccc12]

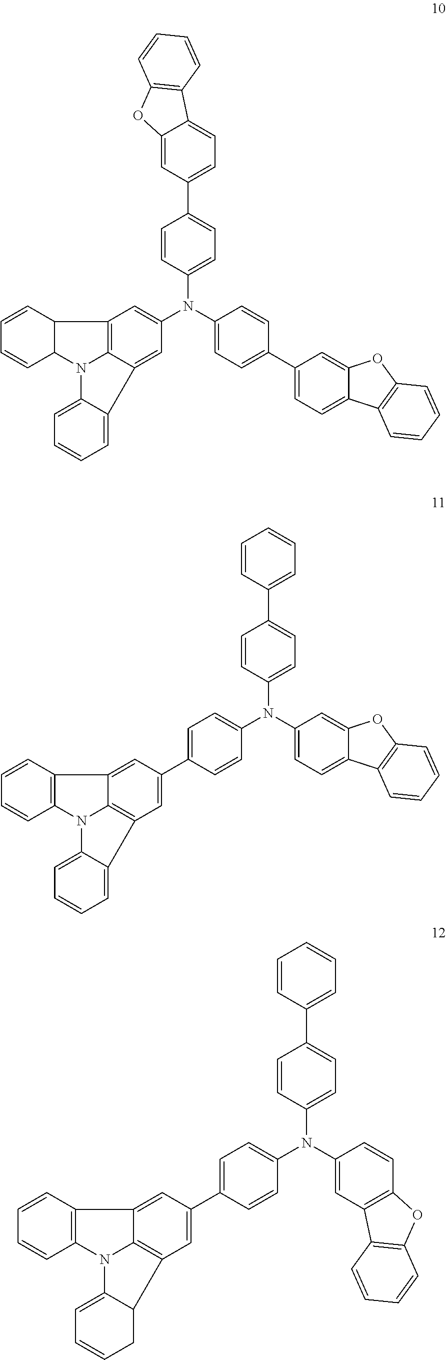 [SMILES: C1=CC2c3cc(N(c4ccc(-c5ccc6c(c5)oc5ccccc56)cc4)c4ccc(-c5ccc6c(c5)oc5ccccc56)cc4)cc4c5ccccc5n(c34)C2C=C1 ; c1ccc(-c2ccc(N(c3ccc(-c4cc5c6ccccc6n6c7ccccc7c(c4)c56)cc3)c3ccc4c(c3)oc3ccccc34)cc2)cc1 ; C1=CCC2C(=C1)n1c3ccccc3c3cc(-c4ccc(N(c5ccc(-c6ccccc6)cc5)c5ccc6oc7ccccc7c6c5)cc4)cc2c31]

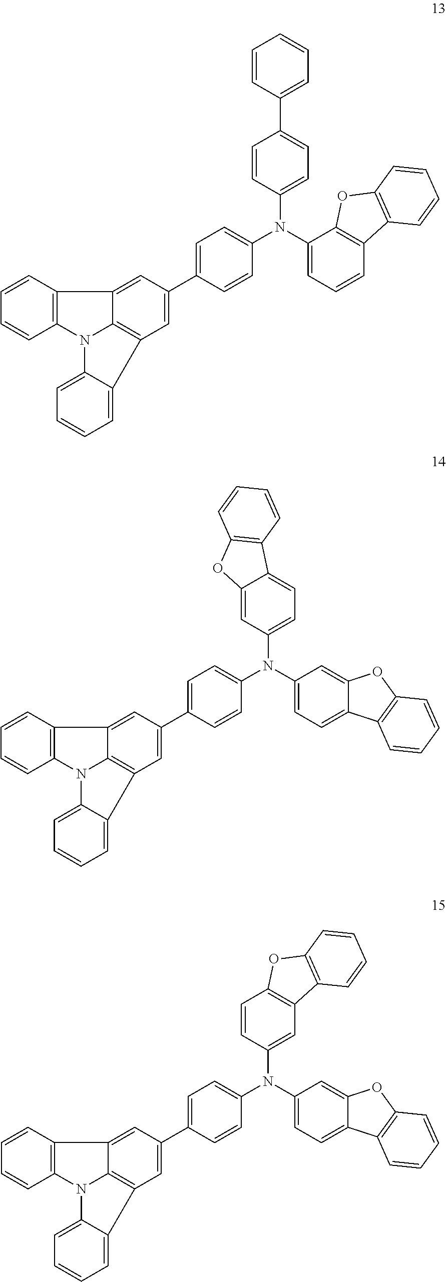 [SMILES: c1ccc(-c2ccc(N(c3ccc(-c4cc5c6ccccc6n6c7ccccc7c(c4)c56)cc3)c3cccc4c3oc3ccccc34)cc2)cc1 ; c1ccc2c(c1)oc1cc(N(c3ccc(-c4cc5c6ccccc6n6c7ccccc7c(c4)c56)cc3)c3ccc4c(c3)oc3ccccc34)ccc12 ; c1ccc2c(c1)oc1cc(N(c3ccc(-c4cc5c6ccccc6n6c7ccccc7c(c4)c56)cc3)c3ccc4oc5ccccc5c4c3)ccc12]

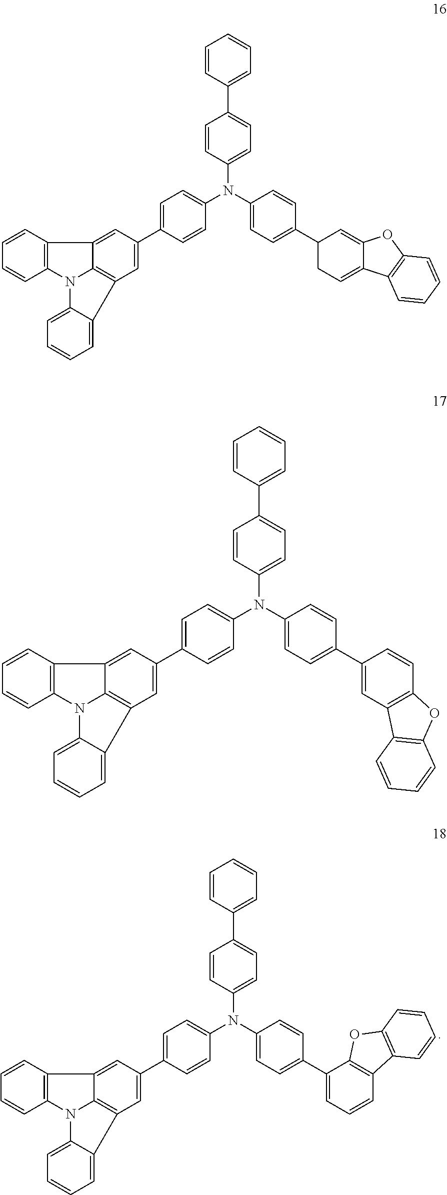 [SMILES: C1=c2oc3ccccc3c2=CCC1c1ccc(N(c2ccc(-c3ccccc3)cc2)c2ccc(-c3cc4c5ccccc5n5c6ccccc6c(c3)c45)cc2)cc1 ; c1ccc(-c2ccc(N(c3ccc(-c4ccc5oc6ccccc6c5c4)cc3)c3ccc(-c4cc5c6ccccc6n6c7ccccc7c(c4)c56)cc3)cc2)cc1 ; c1ccc(-c2ccc(N(c3ccc(-c4cc5c6ccccc6n6c7ccccc7c(c4)c56)cc3)c3ccc(-c4cccc5c4oc4ccccc45)cc3)cc2)cc1]

8. The organic EL device as claimed in claim 4, wherein the material is one of the following Compounds 19 to 36:

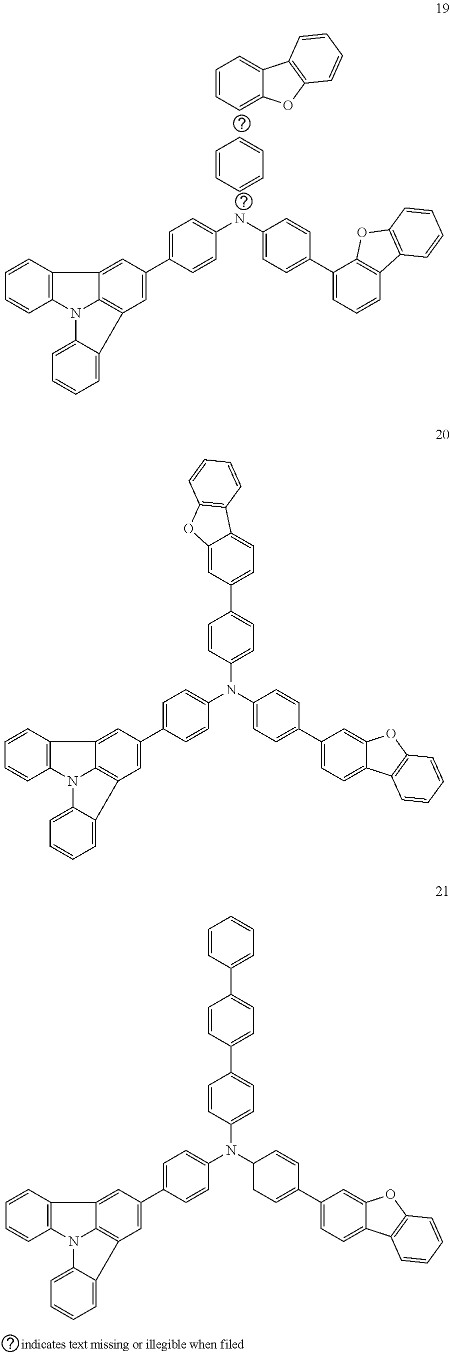

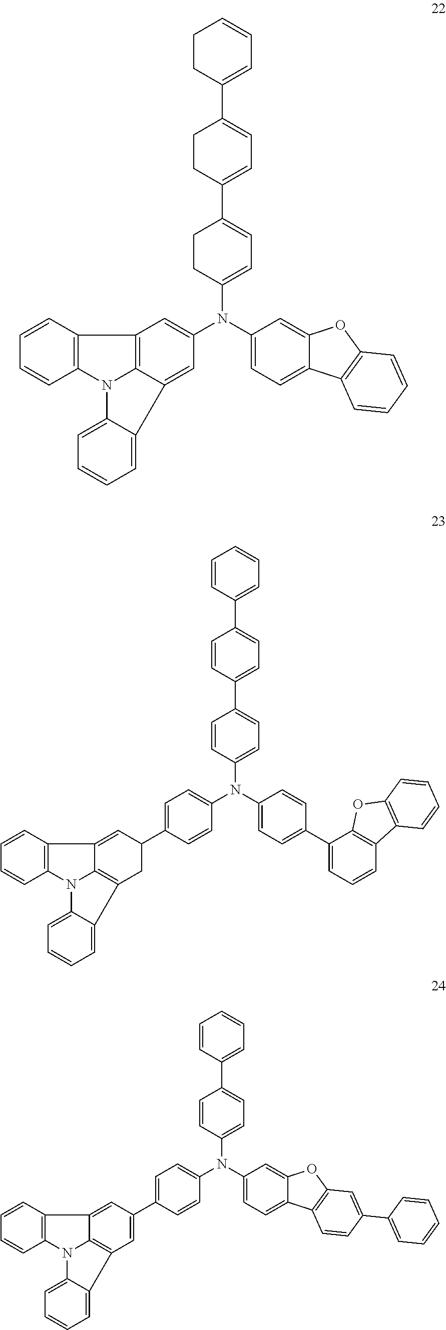 [SMILES: C1=CCCC(C2=CC=C(C3=CC=C(N(c4ccc5c(c4)oc4ccccc45)c4cc5c6ccccc6n6c7ccccc7c(c4)c56)CC3)CC2)=C1 ; C1=c2c3ccccc3n3c2c(c2ccccc23)CC1c1ccc(N(c2ccc(-c3ccc(-c4ccccc4)cc3)cc2)c2ccc(-c3cccc4c3oc3ccccc34)cc2)cc1 ; c1ccc(-c2ccc(N(c3ccc(-c4cc5c6ccccc6n6c7ccccc7c(c4)c56)cc3)c3ccc4c(c3)oc3cc(-c5ccccc5)ccc34)cc2)cc1]

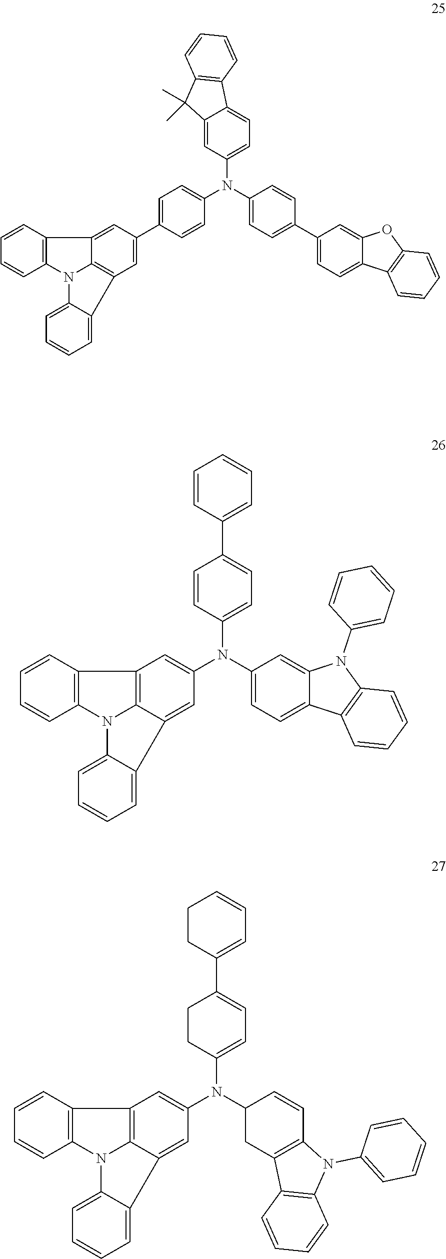 [SMILES: CC1(C)c2ccccc2-c2ccc(N(c3ccc(-c4ccc5c(c4)oc4ccccc45)cc3)c3ccc(-c4cc5c6ccccc6n6c7ccccc7c(c4)c56)cc3)cc21 ; c1ccc(-c2ccc(N(c3cc4c5ccccc5n5c6ccccc6c(c3)c45)c3ccc4c5ccccc5n(-c5ccccc5)c4c3)cc2)cc1 ; C1=CCCC(C2=CC=C(N(c3cc4c5ccccc5n5c6ccccc6c(c3)c45)C3C=Cc4c(c5ccccc5n4-c4ccccc4)C3)CC2)=C1]

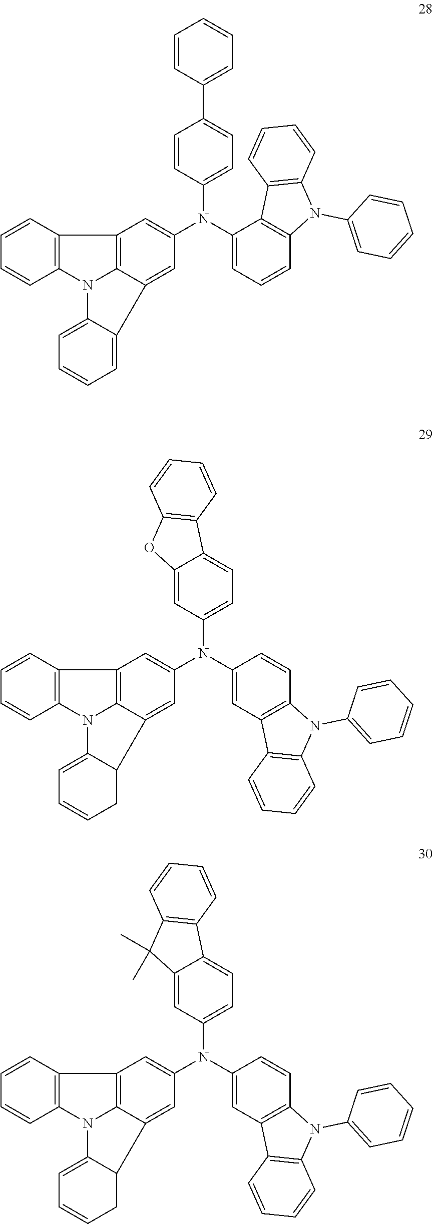 [SMILES: c1ccc(-c2ccc(N(c3cc4c5ccccc5n5c6ccccc6c(c3)c45)c3cccc4c3c3ccccc3n4-c3ccccc3)cc2)cc1 ; C1=CCC2C(=C1)n1c3ccccc3c3cc(N(c4ccc5c(c4)oc4ccccc45)c4ccc5c(c4)c4ccccc4n5-c4ccccc4)cc2c31 ; CC1(C)c2ccccc2-c2ccc(N(c3cc4c5c(c3)c3ccccc3n5C3=CC=CCC34)c3ccc4c(c3)c3ccccc3n4-c3ccccc3)cc21]

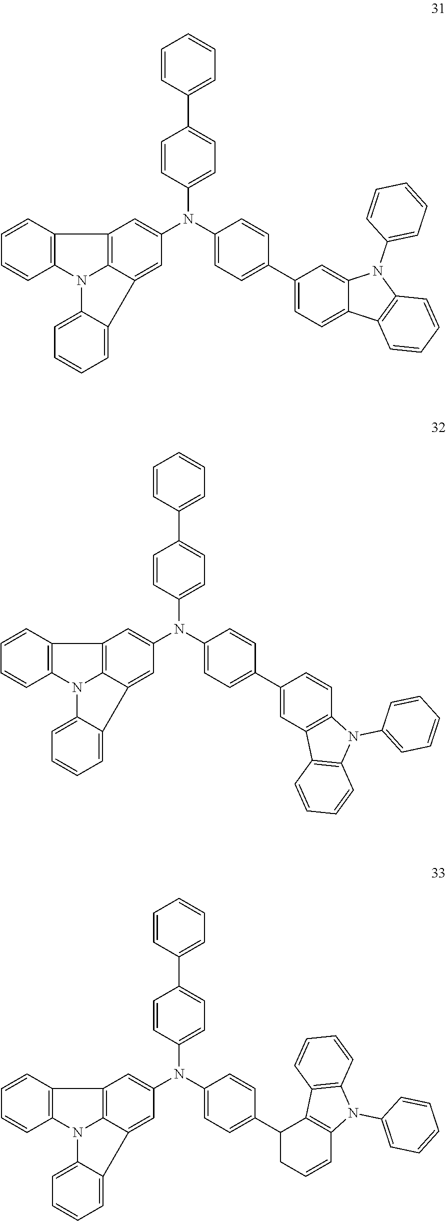 [SMILES: c1ccc(-c2ccc(N(c3ccc(-c4ccc5c6ccccc6n(-c6ccccc6)c5c4)cc3)c3cc4c5ccccc5n5c6ccccc6c(c3)c45)cc2)cc1 ; c1ccc(-c2ccc(N(c3ccc(-c4ccc5c(c4)c4ccccc4n5-c4ccccc4)cc3)c3cc4c5ccccc5n5c6ccccc6c(c3)c45)cc2)cc1 ; C1=Cc2c(c3ccccc3n2-c2ccccc2)C(c2ccc(N(c3ccc(-c4ccccc4)cc3)c3cc4c5ccccc5n5c6ccccc6c(c3)c45)cc2)C1]

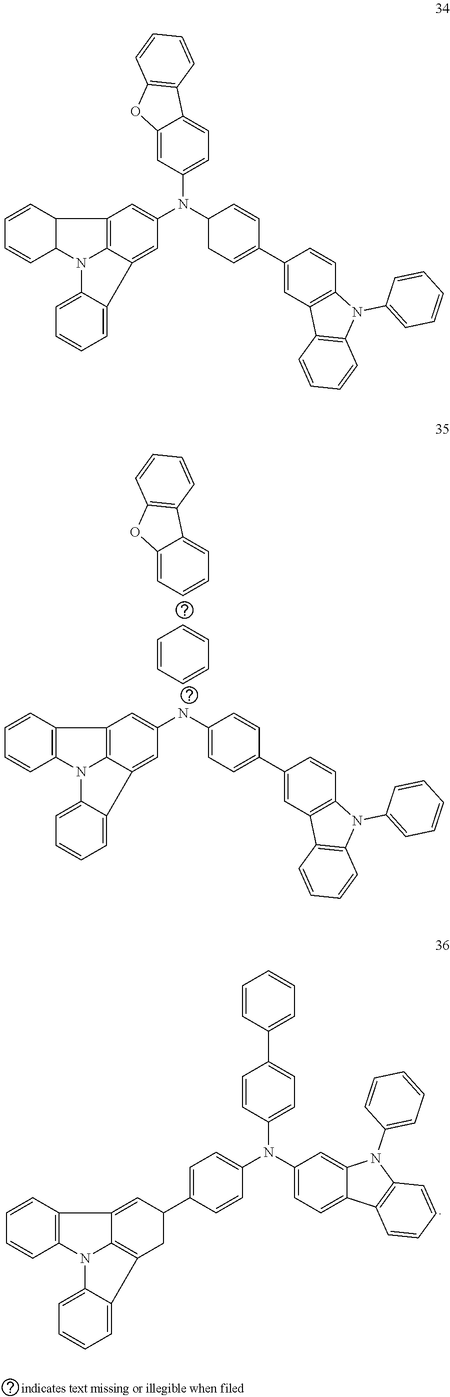

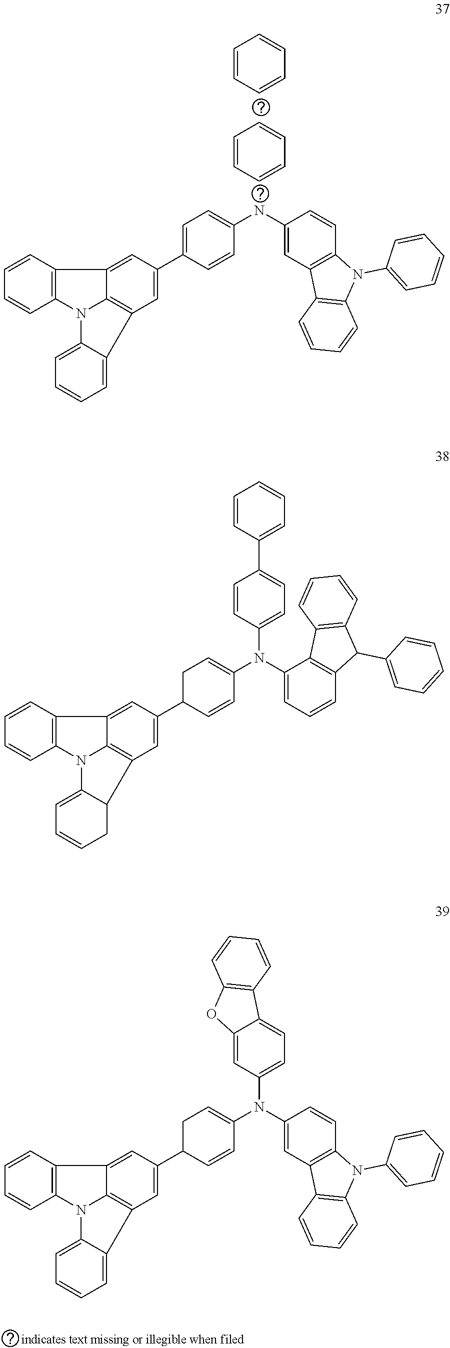

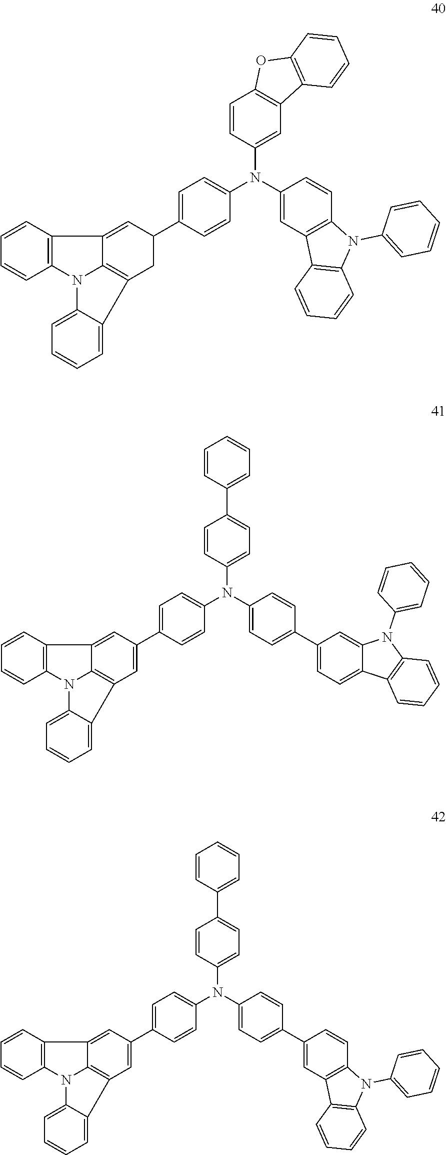 [SMILES: C1=c2c3ccccc3n3c2c(c2ccccc23)CC1c1ccc(N(c2ccc3oc4ccccc4c3c2)c2ccc3c(c2)c2ccccc2n3-c2ccccc2)cc1 ; c1ccc(-c2ccc(N(c3ccc(-c4cc5c6ccccc6n6c7ccccc7c(c4)c56)cc3)c3ccc(-c4ccc5c6ccccc6n(-c6ccccc6)c5c4)cc3)cc2)cc1 ; c1ccc(-c2ccc(N(c3ccc(-c4ccc5c(c4)c4ccccc4n5-c4ccccc4)cc3)c3ccc(-c4cc5c6ccccc6n6c7ccccc7c(c4)c56)cc3)cc2)cc1]

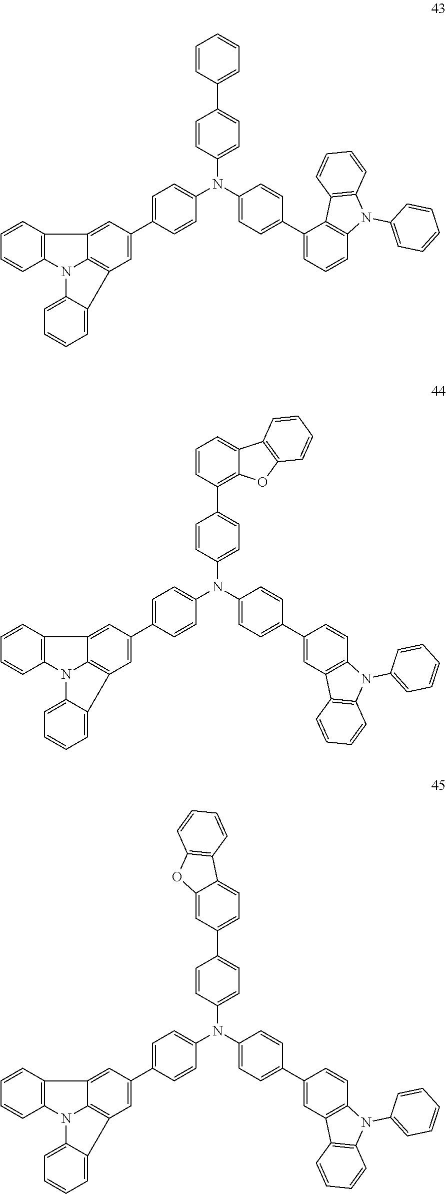 [SMILES: c1ccc(-c2ccc(N(c3ccc(-c4cc5c6ccccc6n6c7ccccc7c(c4)c56)cc3)c3ccc(-c4cccc5c4c4ccccc4n5-c4ccccc4)cc3)cc2)cc1 ; c1ccc(-n2c3ccccc3c3cc(-c4ccc(N(c5ccc(-c6cc7c8ccccc8n8c9ccccc9c(c6)c78)cc5)c5ccc(-c6cccc7c6oc6ccccc67)cc5)cc4)ccc32)cc1 ; c1ccc(-n2c3ccccc3c3cc(-c4ccc(N(c5ccc(-c6ccc7c(c6)oc6ccccc67)cc5)c5ccc(-c6cc7c8ccccc8n8c9ccccc9c(c6)c78)cc5)cc4)ccc32)cc1]

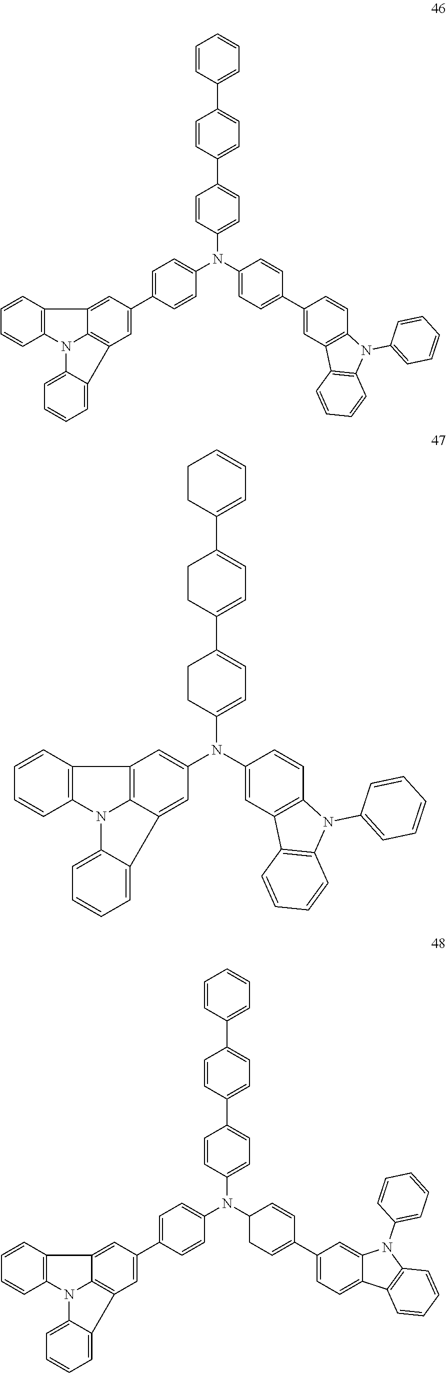 [SMILES: c1ccc(-c2ccc(-c3ccc(N(c4ccc(-c5ccc6c(c5)c5ccccc5n6-c5ccccc5)cc4)c4ccc(-c5cc6c7ccccc7n7c8ccccc8c(c5)c67)cc4)cc3)cc2)cc1 ; C1=CCCC(C2=CC=C(C3=CC=C(N(c4ccc5c(c4)c4ccccc4n5-c4ccccc4)c4cc5c6ccccc6n6c7ccccc7c(c4)c56)CC3)CC2)=C1 ; C1=CC(N(c2ccc(-c3ccc(-c4ccccc4)cc3)cc2)c2ccc(-c3cc4c5ccccc5n5c6ccccc6c(c3)c45)cc2)CC=C1c1ccc2c3ccccc3n(-c3ccccc3)c2c1]

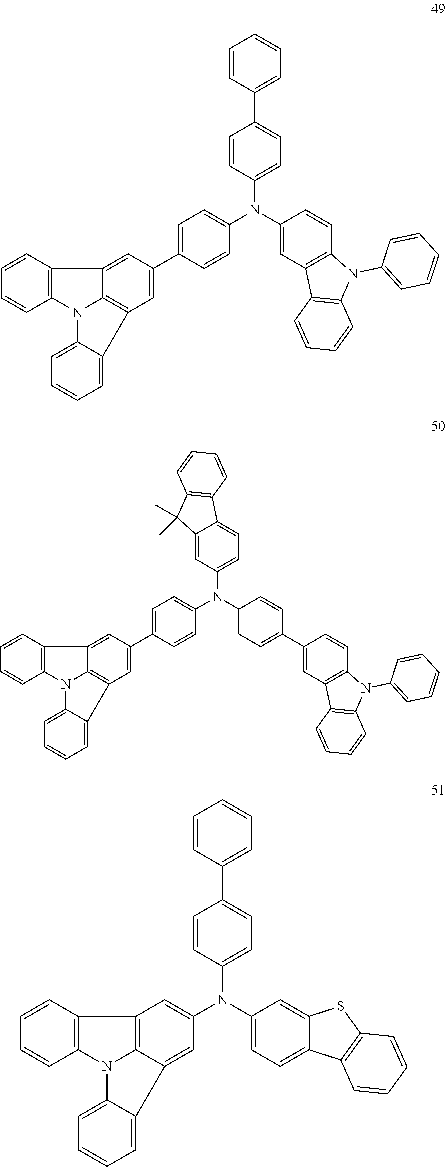 [SMILES: c1ccc(-c2ccc(N(c3ccc(-c4cc5c6ccccc6n6c7ccccc7c(c4)c56)cc3)c3ccc4c(c3)c3ccccc3n4-c3ccccc3)cc2)cc1 ; CC1(C)c2ccccc2-c2ccc(N(c3ccc(-c4cc5c6ccccc6n6c7ccccc7c(c4)c56)cc3)C3C=CC(c4ccc5c(c4)c4ccccc4n5-c4ccccc4)=CC3)cc21 ; c1ccc(-c2ccc(N(c3ccc4c(c3)sc3ccccc34)c3cc4c5ccccc5n5c6ccccc6c(c3)c45)cc2)cc1]

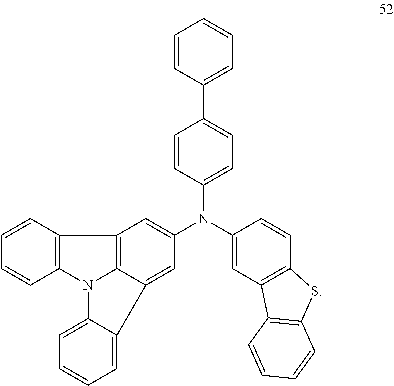 [SMILES: c1ccc(-c2ccc(N(c3ccc4sc5ccccc5c4c3)c3cc4c5ccccc5n5c6ccccc6c(c3)c45)cc2)cc1]

9. The organic EL device as claimed in claim 4, wherein the material is one of the following Compounds 37 to 52:

10. The organic EL device as claimed in claim 4, wherein the material is one of the following Compounds 53 to 70:

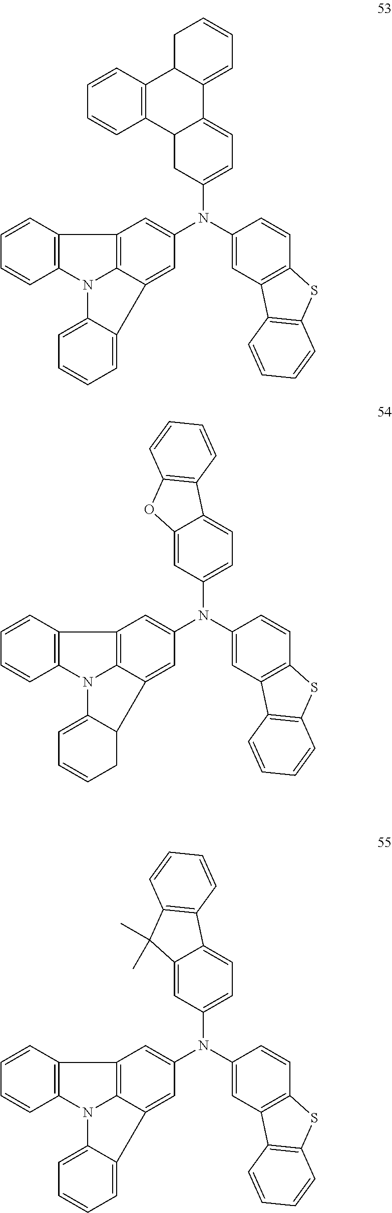 [SMILES: C1=CCC2C(=C1)C1=CC=C(N(c3ccc4sc5ccccc5c4c3)c3cc4c5ccccc5n5c6ccccc6c(c3)c45)CC1c1ccccc12 ; C1=CCC2C(=C1)n1c3ccccc3c3cc(N(c4ccc5c(c4)oc4ccccc45)c4ccc5sc6ccccc6c5c4)cc2c31 ; CC1(C)c2ccccc2-c2ccc(N(c3ccc4sc5ccccc5c4c3)c3cc4c5ccccc5n5c6ccccc6c(c3)c45)cc21]

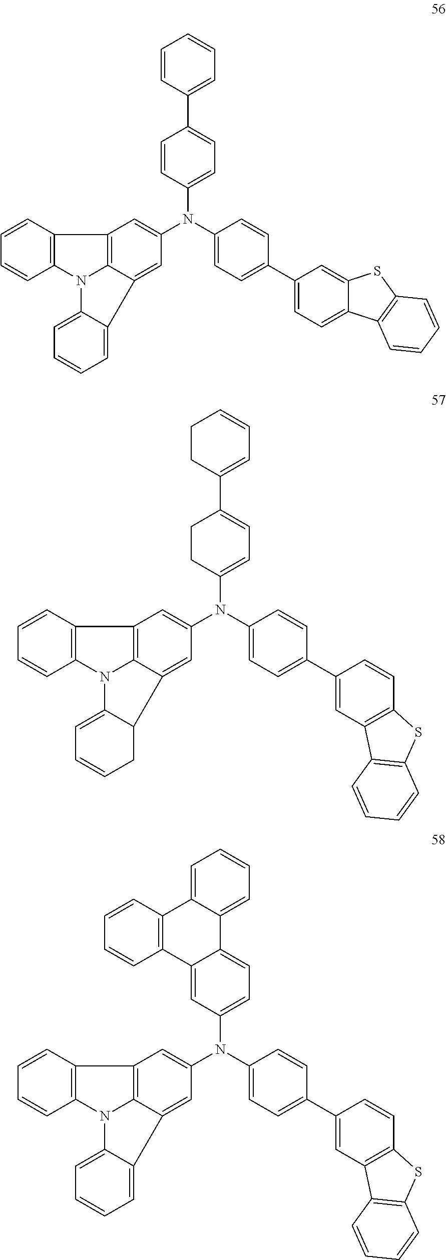 [SMILES: c1ccc(-c2ccc(N(c3ccc(-c4ccc5c(c4)sc4ccccc45)cc3)c3cc4c5ccccc5n5c6ccccc6c(c3)c45)cc2)cc1 ; C1=CCCC(C2=CC=C(N(c3ccc(-c4ccc5sc6ccccc6c5c4)cc3)c3cc4c5c(c3)c3ccccc3n5C3=CC=CCC34)CC2)=C1 ; c1ccc2c(c1)sc1ccc(-c3ccc(N(c4ccc5c6ccccc6c6ccccc6c5c4)c4cc5c6ccccc6n6c7ccccc7c(c4)c56)cc3)cc12]

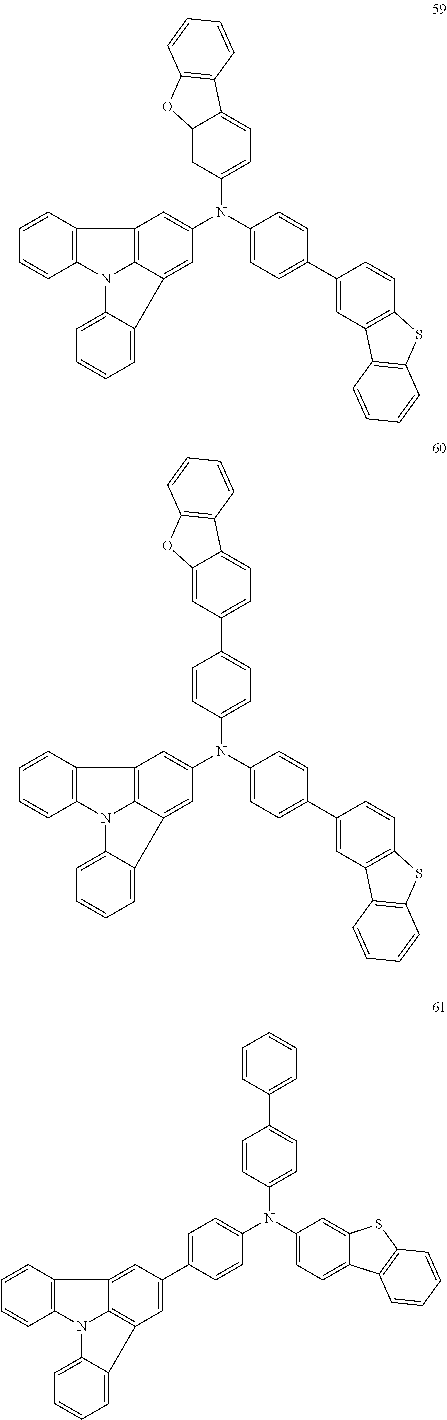 [SMILES: C1=C2c3ccccc3OC2CC(N(c2ccc(-c3ccc4sc5ccccc5c4c3)cc2)c2cc3c4ccccc4n4c5ccccc5c(c2)c34)=C1 ; c1ccc2c(c1)oc1cc(-c3ccc(N(c4ccc(-c5ccc6sc7ccccc7c6c5)cc4)c4cc5c6ccccc6n6c7ccccc7c(c4)c56)cc3)ccc12 ; c1ccc(-c2ccc(N(c3ccc(-c4cc5c6ccccc6n6c7ccccc7c(c4)c56)cc3)c3ccc4c(c3)sc3ccccc34)cc2)cc1]

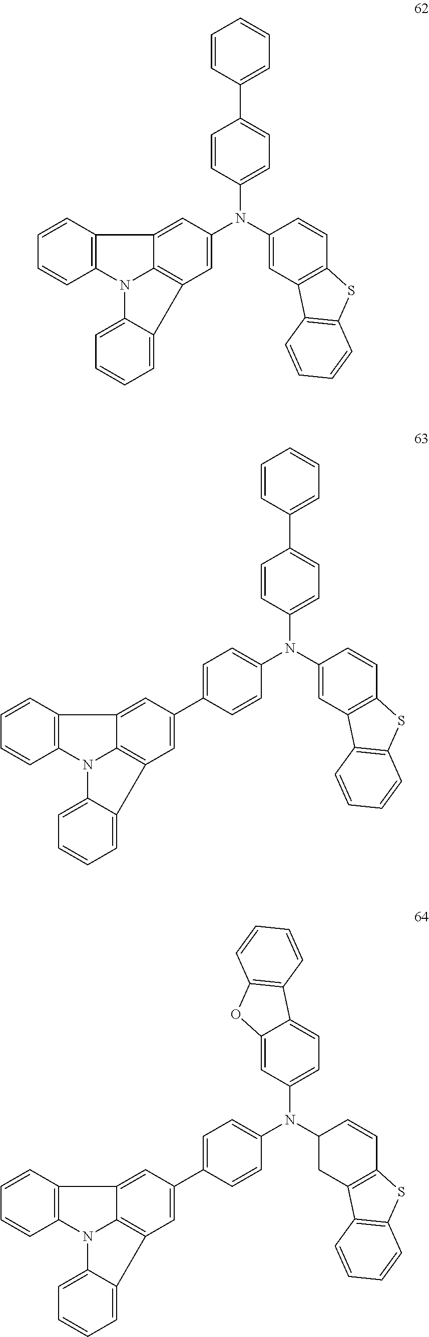 [SMILES: c1ccc(-c2ccc(N(c3ccc4sc5ccccc5c4c3)c3cc4c5ccccc5n5c6ccccc6c(c3)c45)cc2)cc1 ; c1ccc(-c2ccc(N(c3ccc(-c4cc5c6ccccc6n6c7ccccc7c(c4)c56)cc3)c3ccc4sc5ccccc5c4c3)cc2)cc1 ; C1=CC(N(c2ccc(-c3cc4c5ccccc5n5c6ccccc6c(c3)c45)cc2)c2ccc3c(c2)oc2ccccc23)Cc2c1sc1ccccc21]

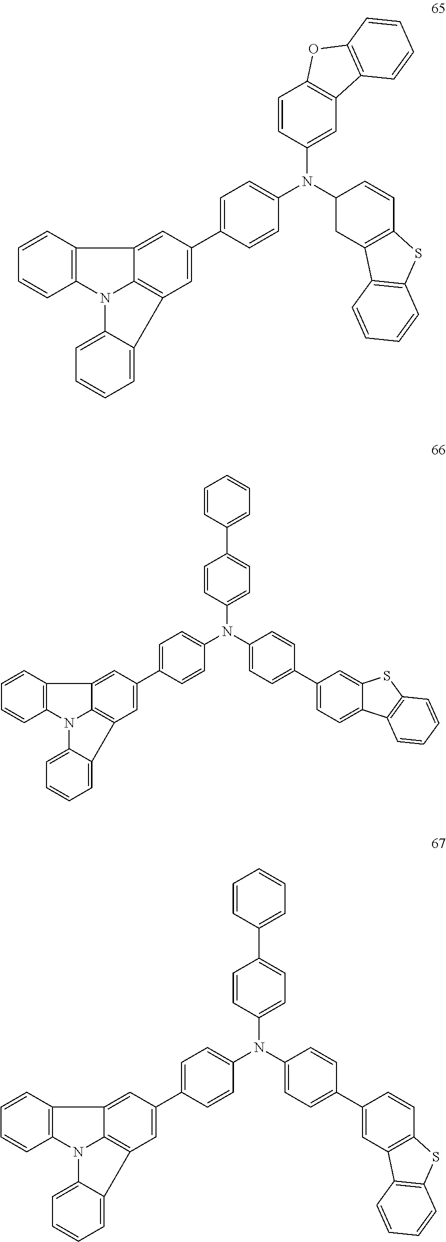 [SMILES: C1=CC(N(c2ccc(-c3cc4c5ccccc5n5c6ccccc6c(c3)c45)cc2)c2ccc3oc4ccccc4c3c2)Cc2c1sc1ccccc21 ; c1ccc(-c2ccc(N(c3ccc(-c4ccc5c(c4)sc4ccccc45)cc3)c3ccc(-c4cc5c6ccccc6n6c7ccccc7c(c4)c56)cc3)cc2)cc1 ; c1ccc(-c2ccc(N(c3ccc(-c4ccc5sc6ccccc6c5c4)cc3)c3ccc(-c4cc5c6ccccc6n6c7ccccc7c(c4)c56)cc3)cc2)cc1]

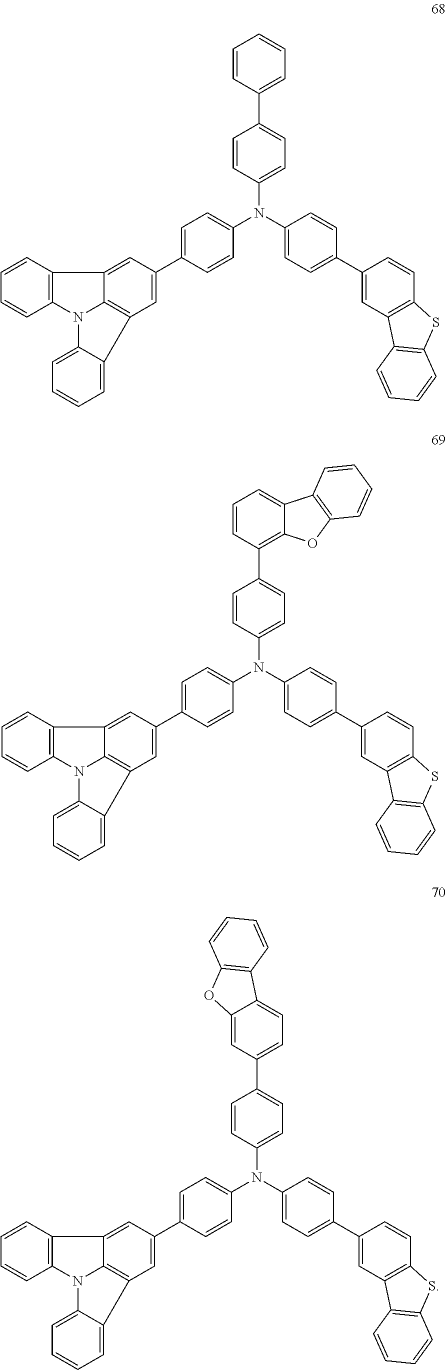 [SMILES: c1ccc(-c2ccc(N(c3ccc(-c4ccc5sc6ccccc6c5c4)cc3)c3ccc(-c4cc5c6ccccc6n6c7ccccc7c(c4)c56)cc3)cc2)cc1 ; c1ccc2c(c1)oc1c(-c3ccc(N(c4ccc(-c5ccc6sc7ccccc7c6c5)cc4)c4ccc(-c5cc6c7ccccc7n7c8ccccc8c(c5)c67)cc4)cc3)cccc12 ; c1ccc2c(c1)oc1cc(-c3ccc(N(c4ccc(-c5ccc6sc7ccccc7c6c5)cc4)c4ccc(-c5cc6c7ccccc7n7c8ccccc8c(c5)c67)cc4)cc3)ccc12]

11. The organic EL device as claimed in claim 4, wherein the material is one of the following Compounds 71 to 88:

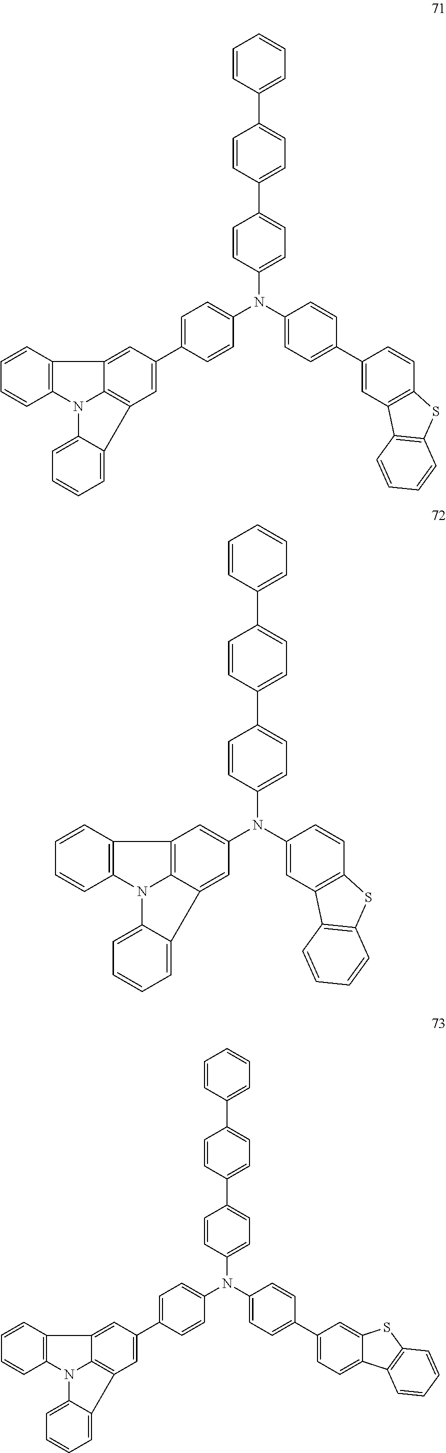 [SMILES: c1ccc(-c2ccc(-c3ccc(N(c4ccc(-c5ccc6sc7ccccc7c6c5)cc4)c4ccc(-c5cc6c7ccccc7n7c8ccccc8c(c5)c67)cc4)cc3)cc2)cc1 ; c1ccc(-c2ccc(-c3ccc(N(c4ccc5sc6ccccc6c5c4)c4cc5c6ccccc6n6c7ccccc7c(c4)c56)cc3)cc2)cc1 ; c1ccc(-c2ccc(-c3ccc(N(c4ccc(-c5ccc6c(c5)sc5ccccc56)cc4)c4ccc(-c5cc6c7ccccc7n7c8ccccc8c(c5)c67)cc4)cc3)cc2)cc1]

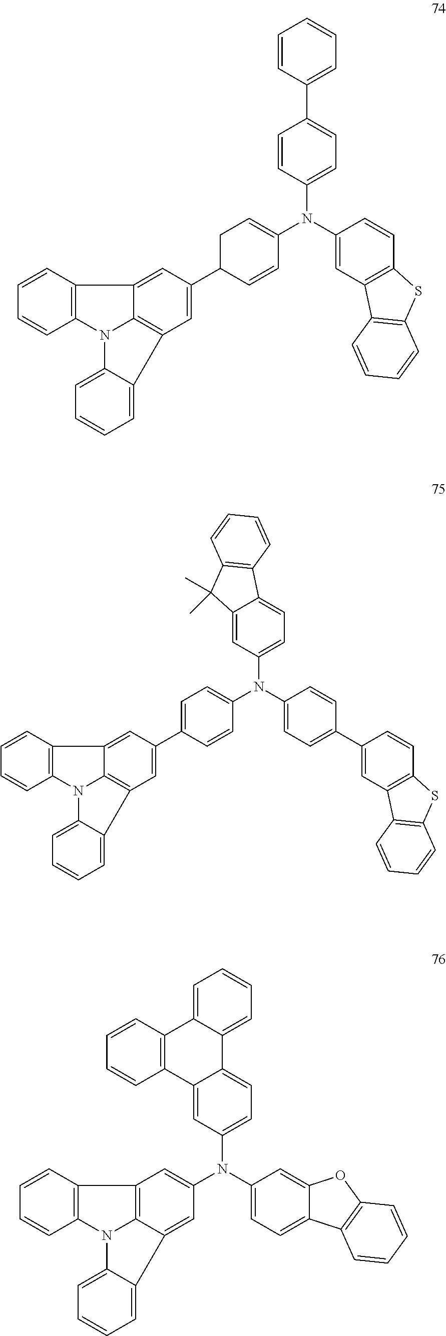 [SMILES: C1=CC(c2cc3c4ccccc4n4c5ccccc5c(c2)c34)CC=C1N(c1ccc(-c2ccccc2)cc1)c1ccc2sc3ccccc3c2c1 ; CC1(C)c2ccccc2-c2ccc(N(c3ccc(-c4ccc5sc6ccccc6c5c4)cc3)c3ccc(-c4cc5c6ccccc6n6c7ccccc7c(c4)c56)cc3)cc21 ; c1ccc2c(c1)oc1cc(N(c3ccc4c5ccccc5c5ccccc5c4c3)c3cc4c5ccccc5n5c6ccccc6c(c3)c45)ccc12]

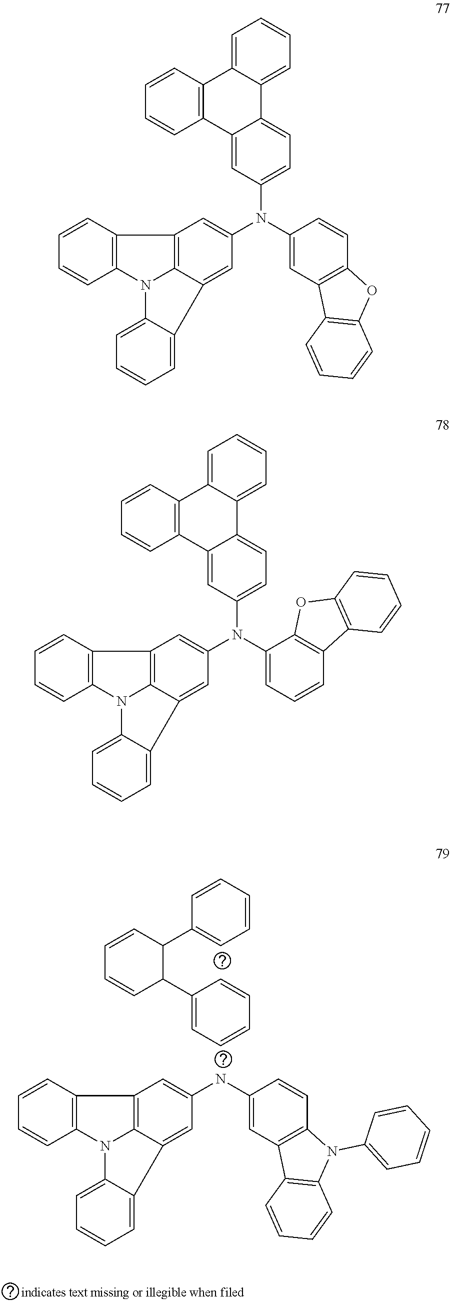

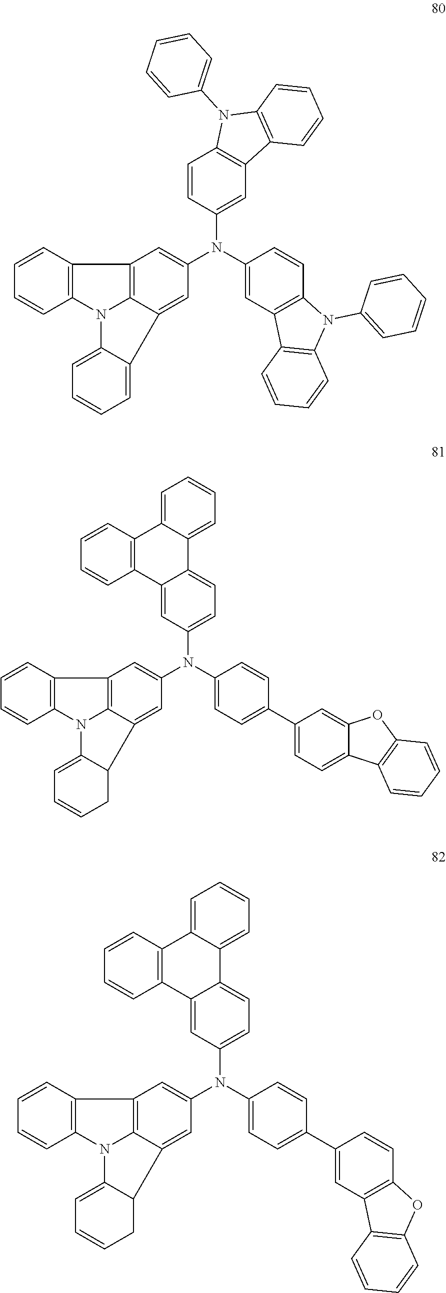 [SMILES: c1ccc(-n2c3ccccc3c3cc(N(c4ccc5c(c4)c4ccccc4n5-c4ccccc4)c4cc5c6ccccc6n6c7ccccc7c(c4)c56)ccc32)cc1 ; C1=CCC2C(=C1)n1c3ccccc3c3cc(N(c4ccc(-c5ccc6c(c5)oc5ccccc56)cc4)c4ccc5c6ccccc6c6ccccc6c5c4)cc2c31 ; C1=CCC2C(=C1)n1c3ccccc3c3cc(N(c4ccc(-c5ccc6oc7ccccc7c6c5)cc4)c4ccc5c6ccccc6c6ccccc6c5c4)cc2c31]

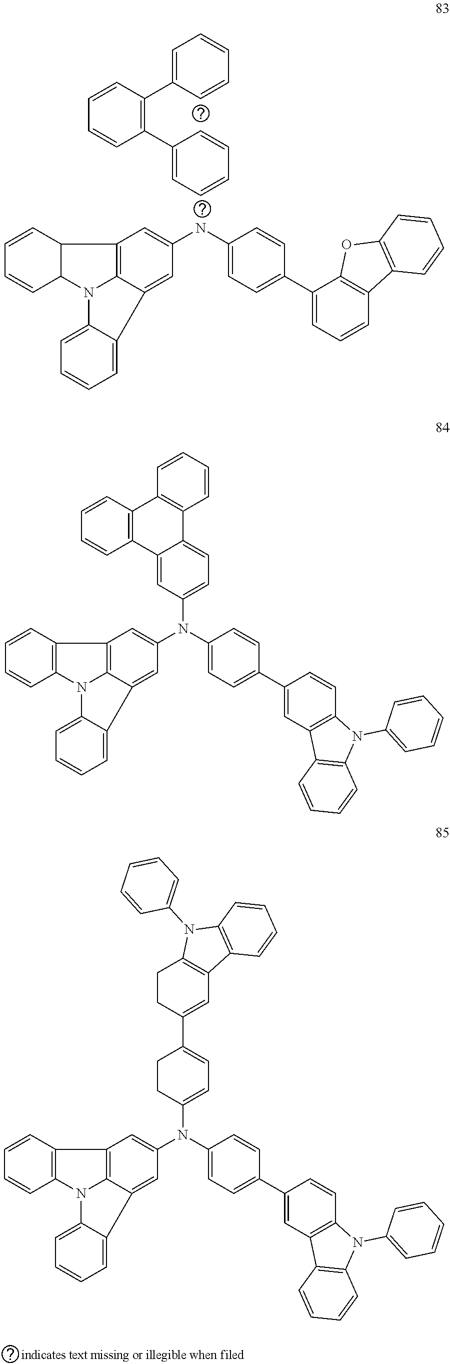

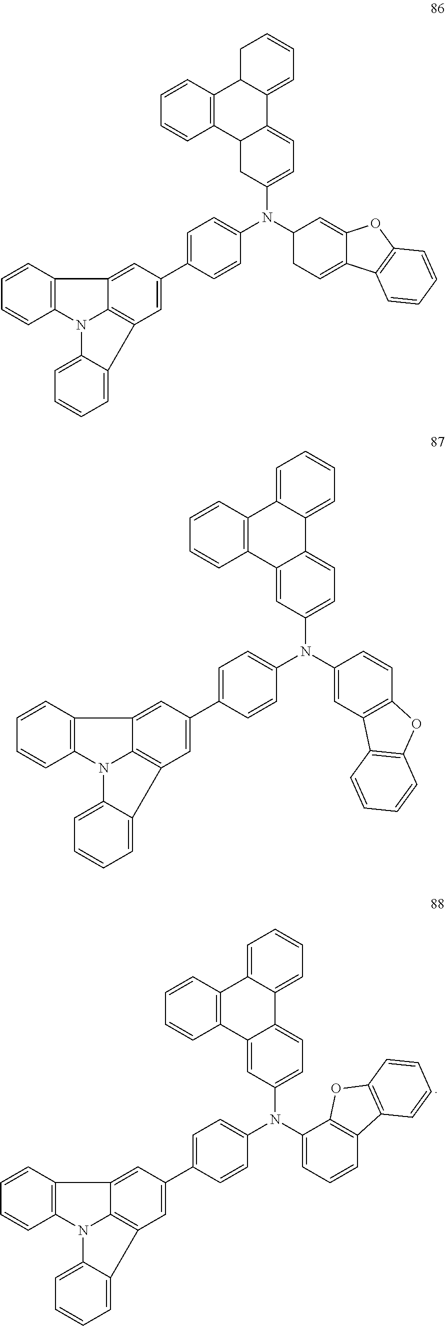 [SMILES: C1=CCC2C(=C1)C1=CC=C(N(c3ccc(-c4cc5c6ccccc6n6c7ccccc7c(c4)c56)cc3)C3C=c4oc5ccccc5c4=CC3)CC1c1ccccc12 ; c1ccc2c(c1)oc1ccc(N(c3ccc(-c4cc5c6ccccc6n6c7ccccc7c(c4)c56)cc3)c3ccc4c5ccccc5c5ccccc5c4c3)cc12 ; c1ccc2c(c1)oc1c(N(c3ccc(-c4cc5c6ccccc6n6c7ccccc7c(c4)c56)cc3)c3ccc4c5ccccc5c5ccccc5c4c3)cccc12]

12. The organic EL device as claimed in claim 4, wherein the material is one of the following Compounds 89 to 104:

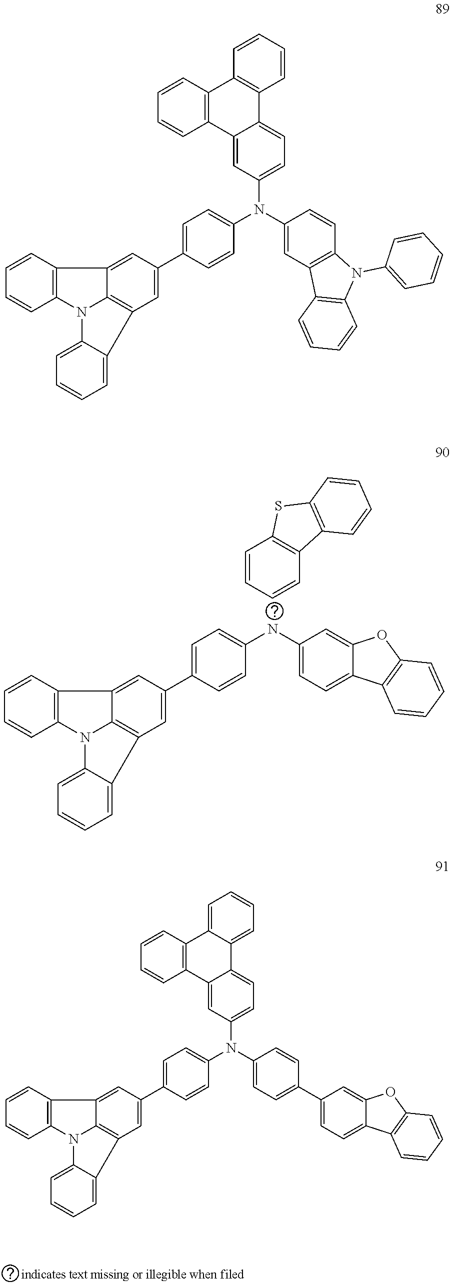

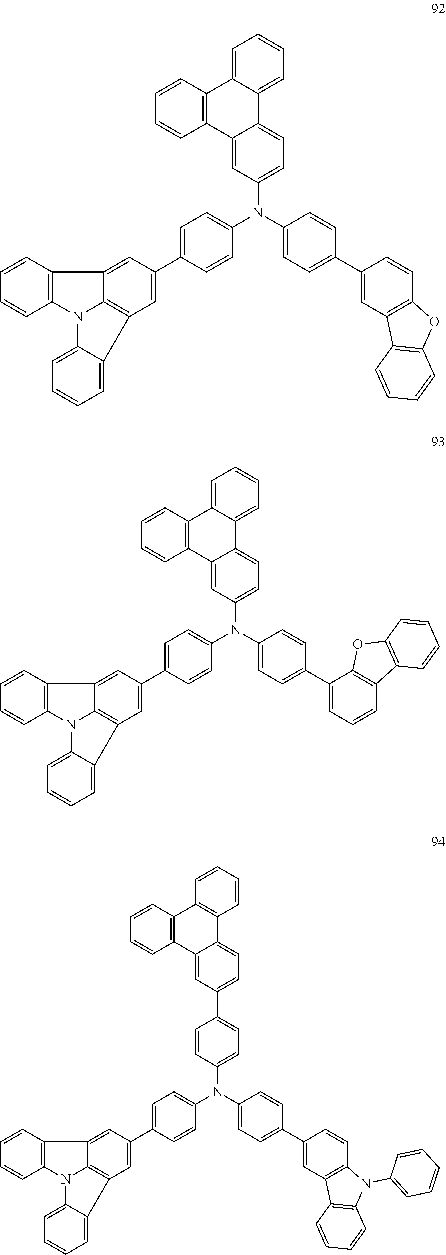 [SMILES: c1ccc2c(c1)oc1ccc(-c3ccc(N(c4ccc(-c5cc6c7ccccc7n7c8ccccc8c(c5)c67)cc4)c4ccc5c6ccccc6c6ccccc6c5c4)cc3)cc12 ; c1ccc2c(c1)oc1c(-c3ccc(N(c4ccc(-c5cc6c7ccccc7n7c8ccccc8c(c5)c67)cc4)c4ccc5c6ccccc6c6ccccc6c5c4)cc3)cccc12 ; c1ccc(-n2c3ccccc3c3cc(-c4ccc(N(c5ccc(-c6ccc7c8ccccc8c8ccccc8c7c6)cc5)c5ccc(-c6cc7c8ccccc8n8c9ccccc9c(c6)c78)cc5)cc4)ccc32)cc1]

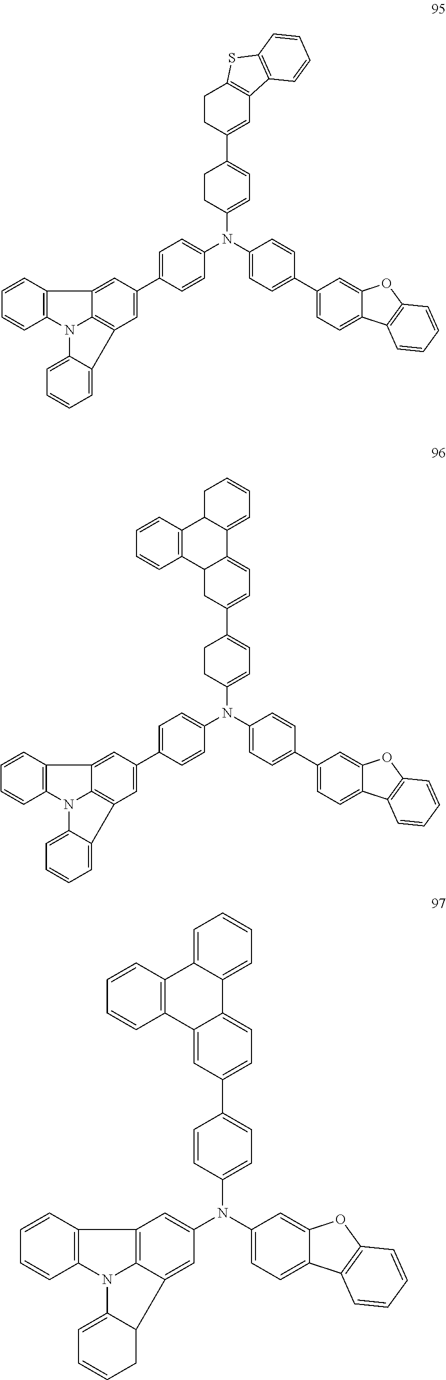 [SMILES: C1=C(C2=Cc3c(sc4ccccc34)CC2)CCC(N(c2ccc(-c3ccc4c(c3)oc3ccccc34)cc2)c2ccc(-c3cc4c5ccccc5n5c6ccccc6c(c3)c45)cc2)=C1 ; C1=CCC2C(=C1)C1=CC=C(C3=CC=C(N(c4ccc(-c5ccc6c(c5)oc5ccccc56)cc4)c4ccc(-c5cc6c7ccccc7n7c8ccccc8c(c5)c67)cc4)CC3)CC1c1ccccc12 ; C1=CCC2C(=C1)n1c3ccccc3c3cc(N(c4ccc(-c5ccc6c7ccccc7c7ccccc7c6c5)cc4)c4ccc5c(c4)oc4ccccc45)cc2c31]

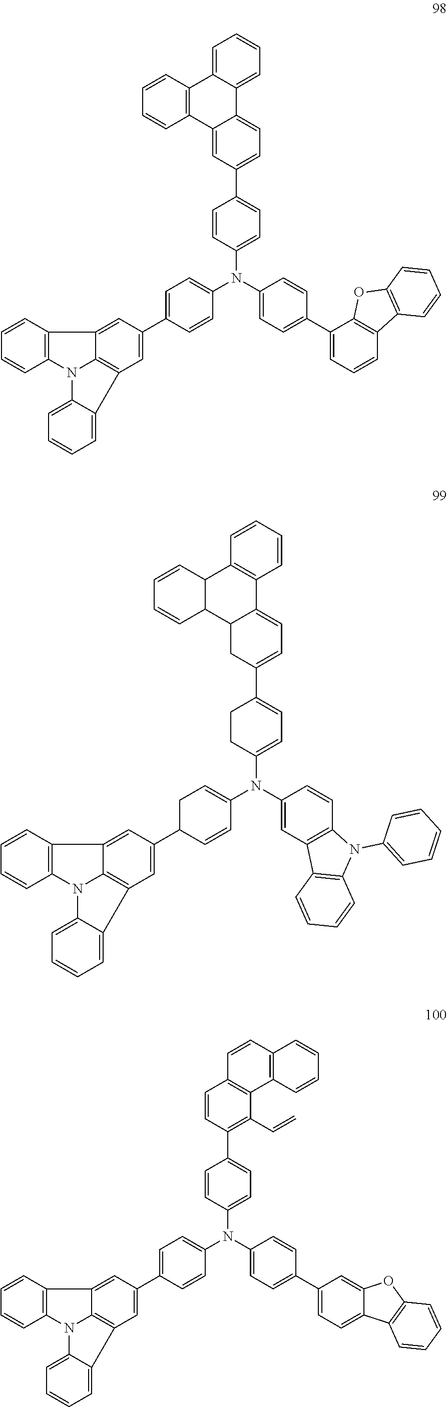 [SMILES: c1ccc2c(c1)oc1c(-c3ccc(N(c4ccc(-c5ccc6c7ccccc7c7ccccc7c6c5)cc4)c4ccc(-c5cc6c7ccccc7n7c8ccccc8c(c5)c67)cc4)cc3)cccc12 ; C1=CC2c3ccccc3C3=CC=C(C4=CC=C(N(C5=CCC(c6cc7c8ccccc8n8c9ccccc9c(c6)c78)C=C5)c5ccc6c(c5)c5ccccc5n6-c5ccccc5)CC4)CC3C2C=C1 ; C=Cc1c(-c2ccc(N(c3ccc(-c4ccc5c(c4)oc4ccccc45)cc3)c3ccc(-c4cc5c6ccccc6n6c7ccccc7c(c4)c56)cc3)cc2)ccc2ccc3ccccc3c12]

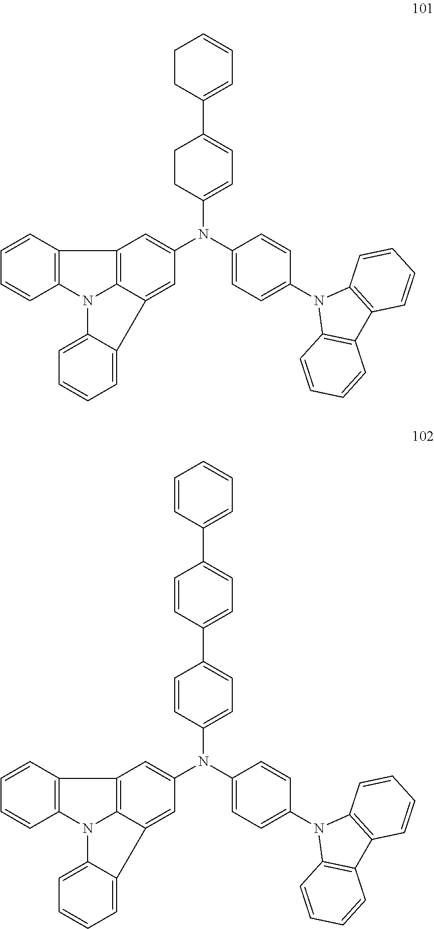 [SMILES: C1=CCCC(C2=CC=C(N(c3ccc(-n4c5ccccc5c5ccccc54)cc3)c3cc4c5ccccc5n5c6ccccc6c(c3)c45)CC2)=C1 ; c1ccc(-c2ccc(-c3ccc(N(c4ccc(-n5c6ccccc6c6ccccc65)cc4)c4cc5c6ccccc6n6c7ccccc7c(c4)c56)cc3)cc2)cc1]

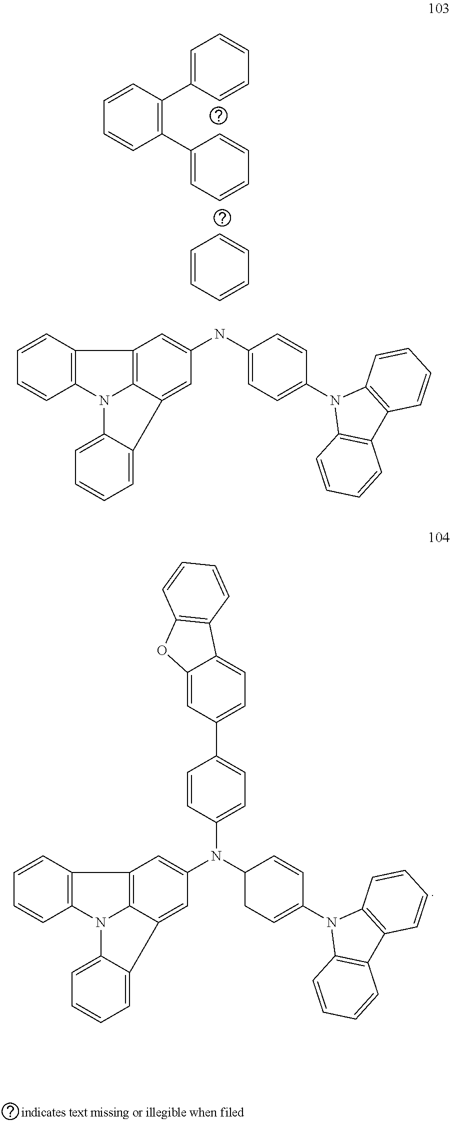

13. The organic EL device as claimed in claim 4, wherein the material is one of the following Compounds 105 to 119:

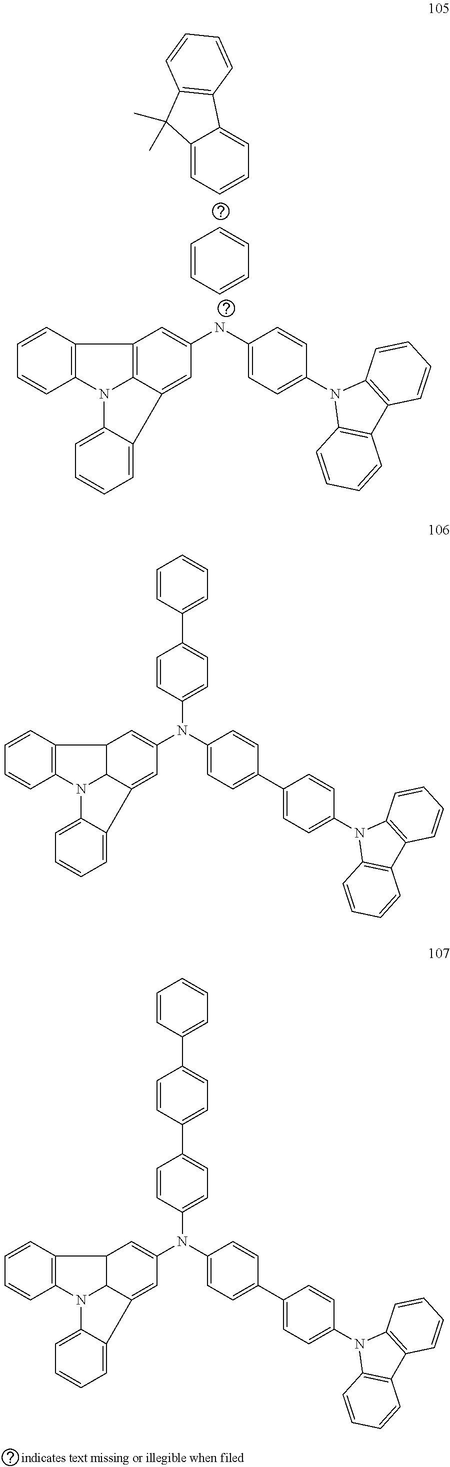

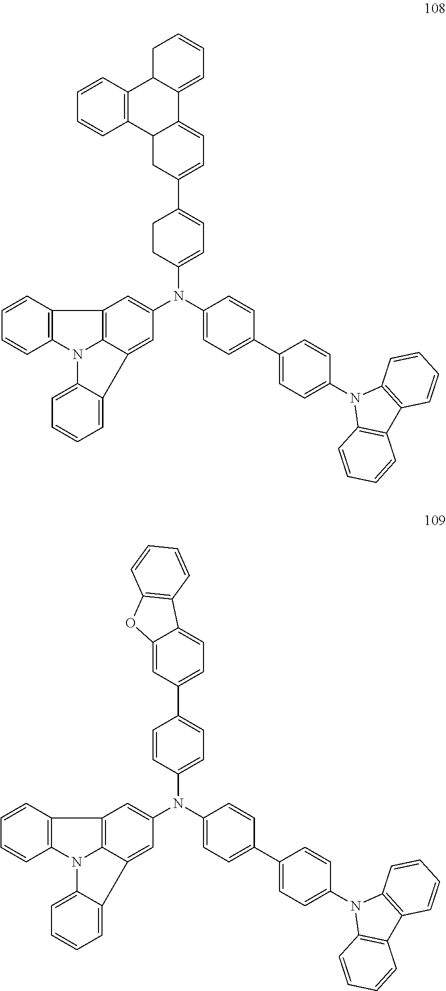 [SMILES: C1=CCC2C(=C1)C1=CC=C(C3=CC=C(N(c4ccc(-c5ccc(-n6c7ccccc7c7ccccc76)cc5)cc4)c4cc5c6ccccc6n6c7ccccc7c(c4)c56)CC3)CC1c1ccccc12 ; c1ccc2c(c1)oc1cc(-c3ccc(N(c4ccc(-c5ccc(-n6c7ccccc7c7ccccc76)cc5)cc4)c4cc5c6ccccc6n6c7ccccc7c(c4)c56)cc3)ccc12]

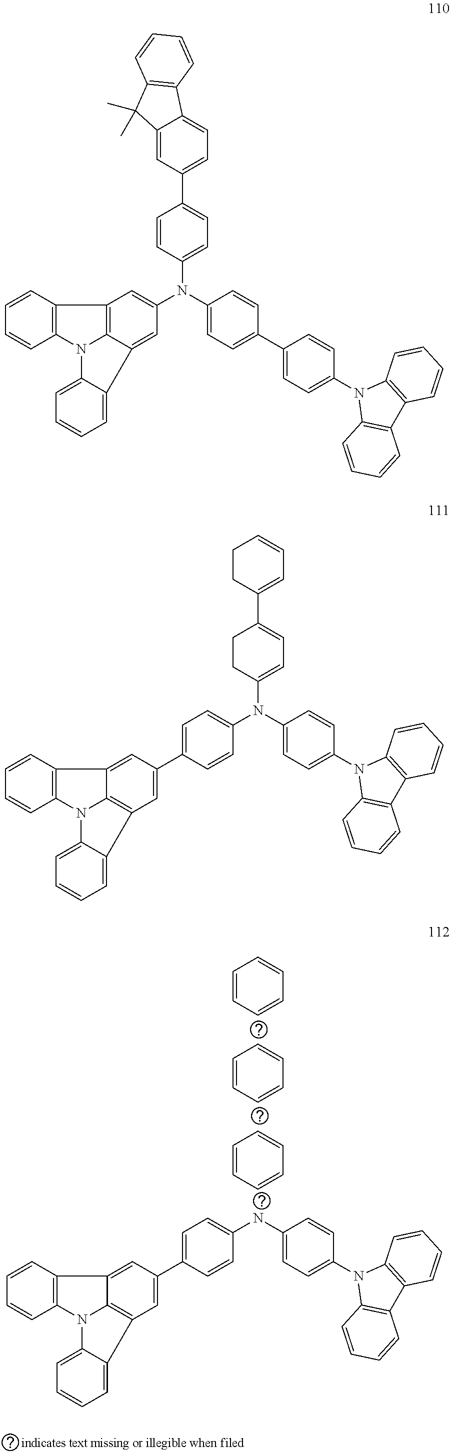

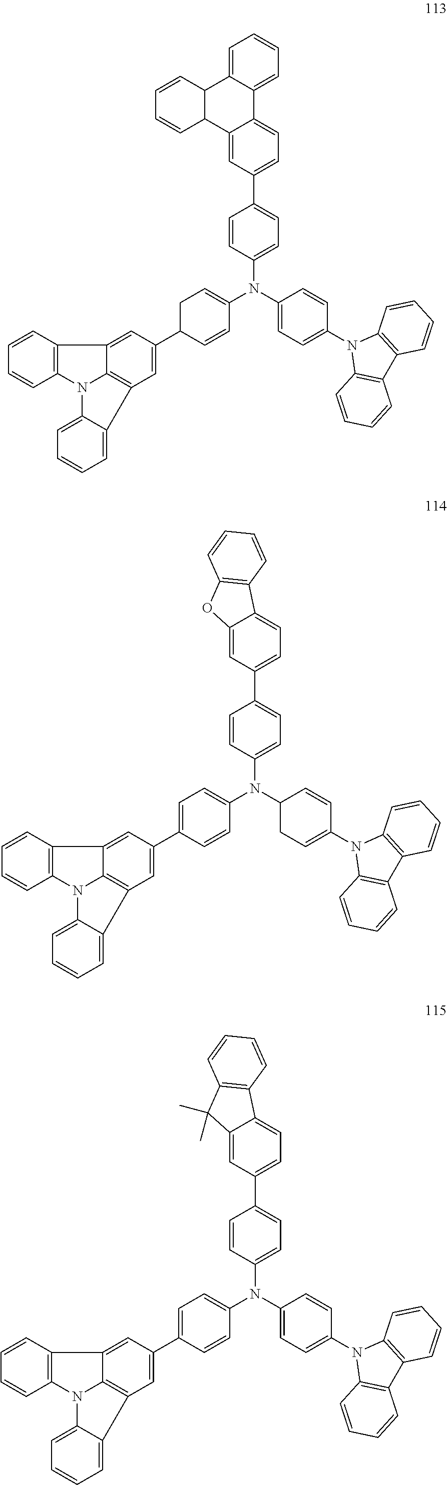 [SMILES: C1=CC2c3ccccc3-c3ccc(-c4ccc(N(C5=CCC(c6cc7c8ccccc8n8c9ccccc9c(c6)c78)C=C5)c5ccc(-n6c7ccccc7c7ccccc76)cc5)cc4)cc3C2C=C1 ; C1=CC(N(c2ccc(-c3ccc4c(c3)oc3ccccc34)cc2)c2ccc(-c3cc4c5ccccc5n5c6ccccc6c(c3)c45)cc2)CC=C1n1c2ccccc2c2ccccc21 ; CC1(C)c2ccccc2-c2ccc(-c3ccc(N(c4ccc(-c5cc6c7ccccc7n7c8ccccc8c(c5)c67)cc4)c4ccc(-n5c6ccccc6c6ccccc65)cc4)cc3)cc21]

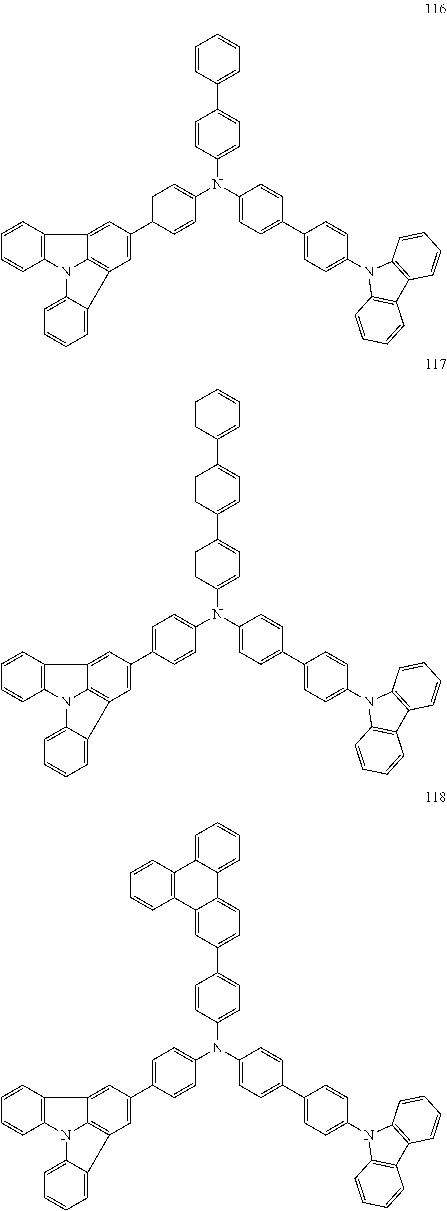 [SMILES: C1=CC(c2cc3c4ccccc4n4c5ccccc5c(c2)c34)CC=C1N(c1ccc(-c2ccccc2)cc1)c1ccc(-c2ccc(-n3c4ccccc4c4ccccc43)cc2)cc1 ; C1=CCCC(C2=CC=C(C3=CC=C(N(c4ccc(-c5ccc(-n6c7ccccc7c7ccccc76)cc5)cc4)c4ccc(-c5cc6c7ccccc7n7c8ccccc8c(c5)c67)cc4)CC3)CC2)=C1 ; c1ccc2c(c1)c1ccccc1c1cc(-c3ccc(N(c4ccc(-c5ccc(-n6c7ccccc7c7ccccc76)cc5)cc4)c4ccc(-c5cc6c7ccccc7n7c8ccccc8c(c5)c67)cc4)cc3)ccc21]

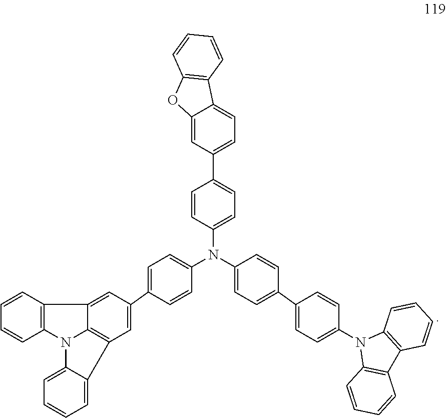 [SMILES: c1ccc2c(c1)oc1cc(-c3ccc(N(c4ccc(-c5ccc(-n6c7ccccc7c7ccccc76)cc5)cc4)c4ccc(-c5cc6c7ccccc7n7c8ccccc8c(c5)c67)cc4)cc3)ccc12]

14. The organic EL device as claimed in claim 4, wherein the material is one of the following Compounds 120 to 134:

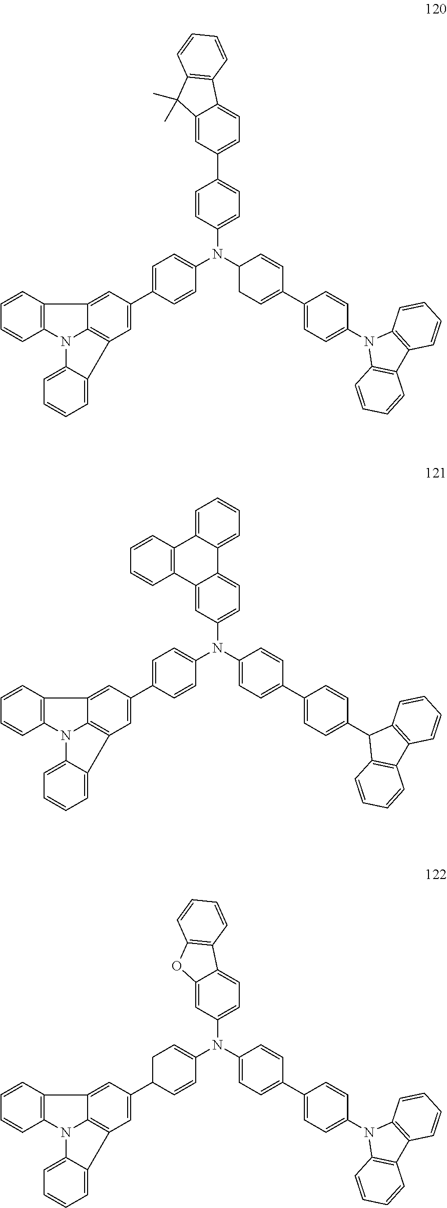 [SMILES: CC1(C)c2ccccc2-c2ccc(-c3ccc(N(c4ccc(-c5cc6c7ccccc7n7c8ccccc8c(c5)c67)cc4)C4C=CC(c5ccc(-n6c7ccccc7c7ccccc76)cc5)=CC4)cc3)cc21 ; c1ccc2c(c1)-c1ccccc1C2c1ccc(-c2ccc(N(c3ccc(-c4cc5c6ccccc6n6c7ccccc7c(c4)c56)cc3)c3ccc4c5ccccc5c5ccccc5c4c3)cc2)cc1 ; C1=CC(c2cc3c4ccccc4n4c5ccccc5c(c2)c34)CC=C1N(c1ccc(-c2ccc(-n3c4ccccc4c4ccccc43)cc2)cc1)c1ccc2c(c1)oc1ccccc12]

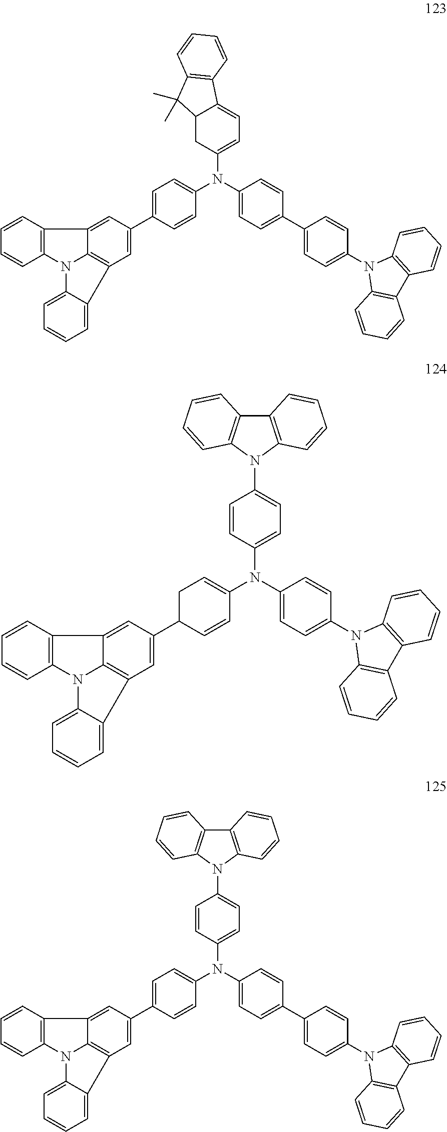 [SMILES: CC1(C)c2ccccc2C2=CC=C(N(c3ccc(-c4ccc(-n5c6ccccc6c6ccccc65)cc4)cc3)c3ccc(-c4cc5c6ccccc6n6c7ccccc7c(c4)c56)cc3)CC21 ; C1=CC(c2cc3c4ccccc4n4c5ccccc5c(c2)c34)CC=C1N(c1ccc(-n2c3ccccc3c3ccccc32)cc1)c1ccc(-n2c3ccccc3c3ccccc32)cc1 ; c1ccc2c(c1)c1ccccc1n2-c1ccc(-c2ccc(N(c3ccc(-c4cc5c6ccccc6n6c7ccccc7c(c4)c56)cc3)c3ccc(-n4c5ccccc5c5ccccc54)cc3)cc2)cc1]

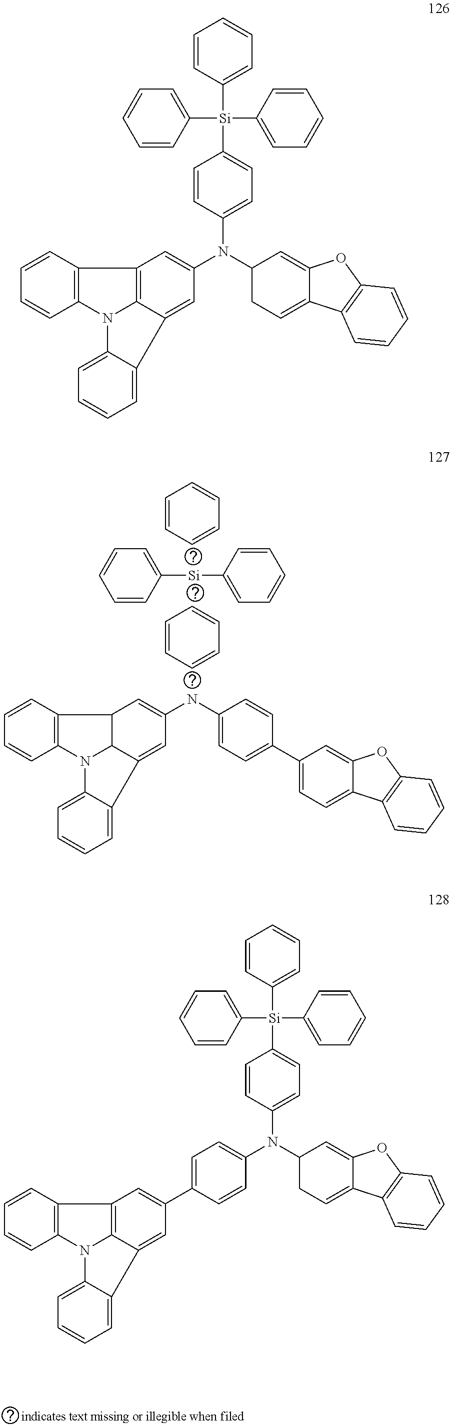

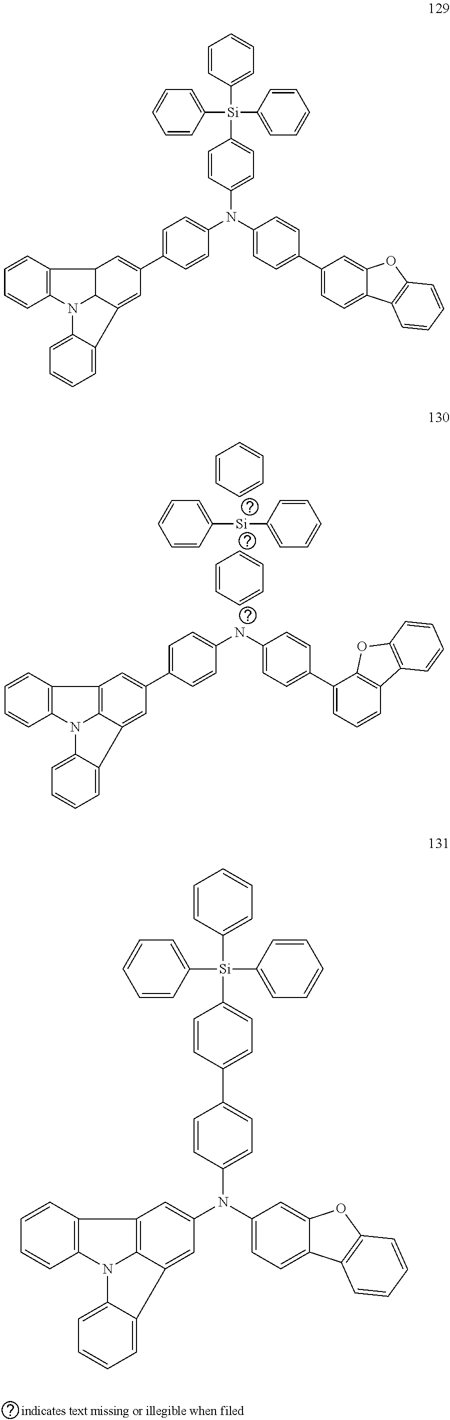

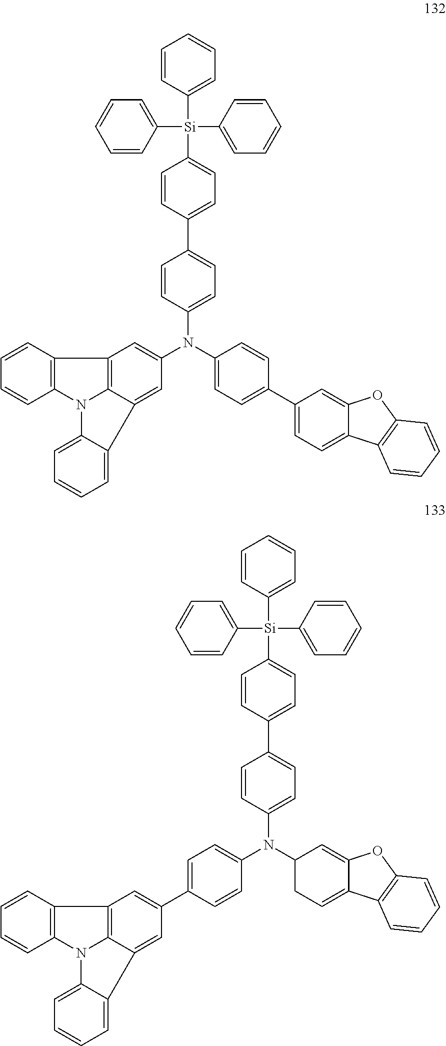 [SMILES: c1ccc([Si](c2ccccc2)(c2ccccc2)c2ccc(-c3ccc(N(c4ccc(-c5ccc6c(c5)oc5ccccc56)cc4)c4cc5c6ccccc6n6c7ccccc7c(c4)c56)cc3)cc2)cc1 ; C1=c2oc3ccccc3c2=CCC1N(c1ccc(-c2ccc([Si](c3ccccc3)(c3ccccc3)c3ccccc3)cc2)cc1)c1ccc(-c2cc3c4ccccc4n4c5ccccc5c(c2)c34)cc1]

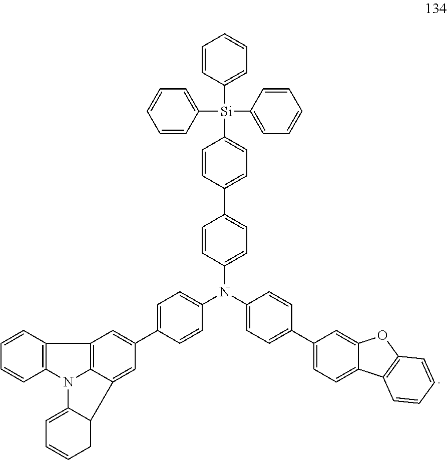 [SMILES: C1=CCC2C(=C1)n1c3ccccc3c3cc(-c4ccc(N(c5ccc(-c6ccc([Si](c7ccccc7)(c7ccccc7)c7ccccc7)cc6)cc5)c5ccc(-c6ccc7c(c6)oc6ccccc67)cc5)cc4)cc2c31]

15. The organic EL device as claimed in claim 4, wherein the material is one of the following Compounds 135 to 148:

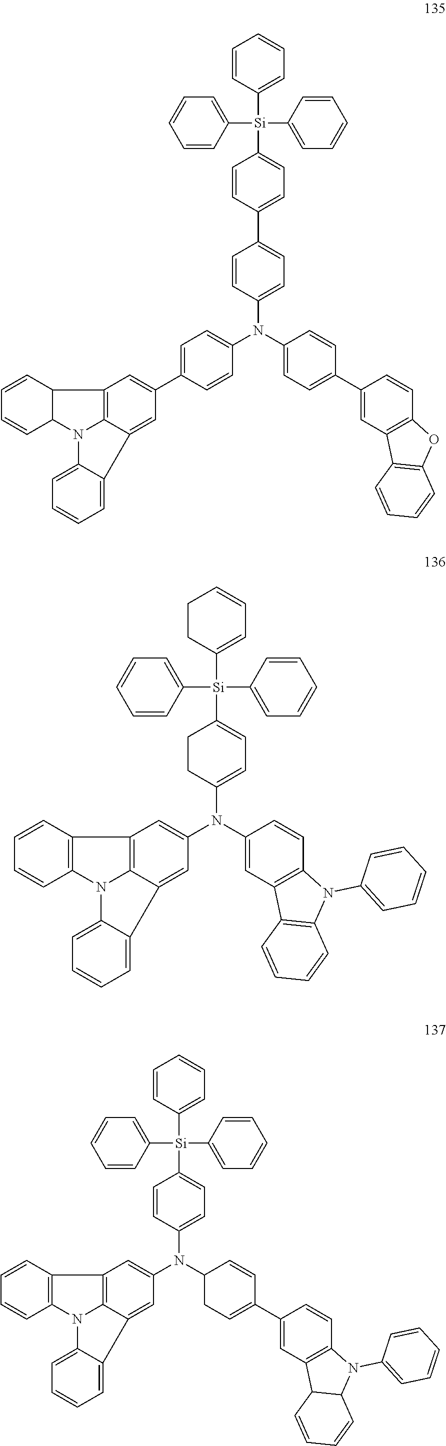 [SMILES: C1=CC2c3cc(-c4ccc(N(c5ccc(-c6ccc([Si](c7ccccc7)(c7ccccc7)c7ccccc7)cc6)cc5)c5ccc(-c6ccc7oc8ccccc8c7c6)cc5)cc4)cc4c5ccccc5n(c34)C2C=C1 ; C1=CCCC([Si](C2=CC=C(N(c3ccc4c(c3)c3ccccc3n4-c3ccccc3)c3cc4c5ccccc5n5c6ccccc6c(c3)c45)CC2)(c2ccccc2)c2ccccc2)=C1 ; C1=CC2c3cc(C4=CCC(N(c5ccc([Si](c6ccccc6)(c6ccccc6)c6ccccc6)cc5)c5cc6c7ccccc7n7c8ccccc8c(c5)c67)C=C4)ccc3N(c3ccccc3)C2C=C1]

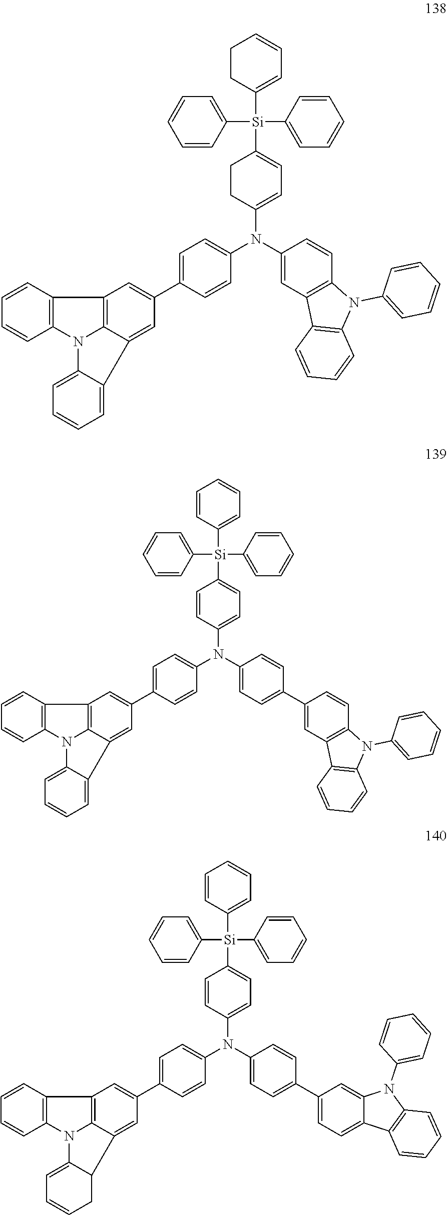 [SMILES: C1=CCCC([Si](C2=CC=C(N(c3ccc(-c4cc5c6ccccc6n6c7ccccc7c(c4)c56)cc3)c3ccc4c(c3)c3ccccc3n4-c3ccccc3)CC2)(c2ccccc2)c2ccccc2)=C1 ; c1ccc(-n2c3ccccc3c3cc(-c4ccc(N(c5ccc(-c6cc7c8ccccc8n8c9ccccc9c(c6)c78)cc5)c5ccc([Si](c6ccccc6)(c6ccccc6)c6ccccc6)cc5)cc4)ccc32)cc1 ; C1=CCC2C(=C1)n1c3ccccc3c3cc(-c4ccc(N(c5ccc(-c6ccc7c8ccccc8n(-c8ccccc8)c7c6)cc5)c5ccc([Si](c6ccccc6)(c6ccccc6)c6ccccc6)cc5)cc4)cc2c31]

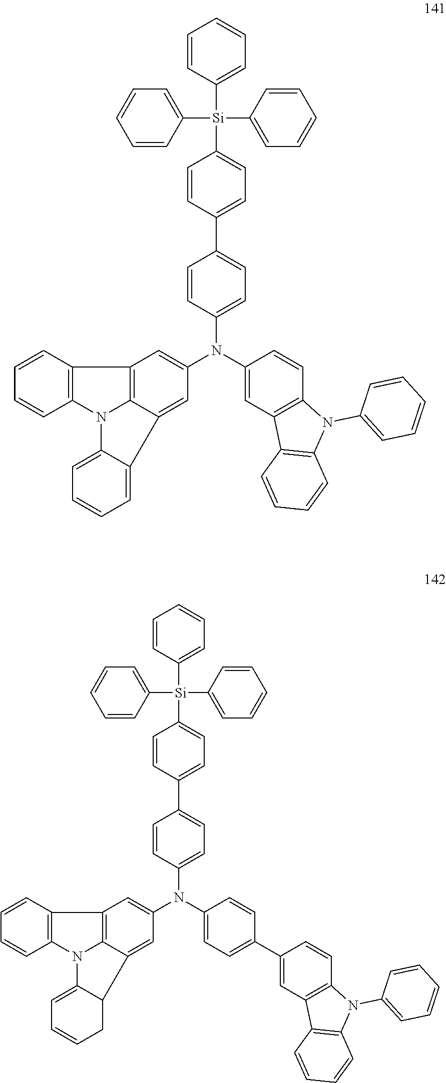 [SMILES: c1ccc(-n2c3ccccc3c3cc(N(c4ccc(-c5ccc([Si](c6ccccc6)(c6ccccc6)c6ccccc6)cc5)cc4)c4cc5c6ccccc6n6c7ccccc7c(c4)c56)ccc32)cc1 ; C1=CCC2C(=C1)n1c3ccccc3c3cc(N(c4ccc(-c5ccc([Si](c6ccccc6)(c6ccccc6)c6ccccc6)cc5)cc4)c4ccc(-c5ccc6c(c5)c5ccccc5n6-c5ccccc5)cc4)cc2c31]

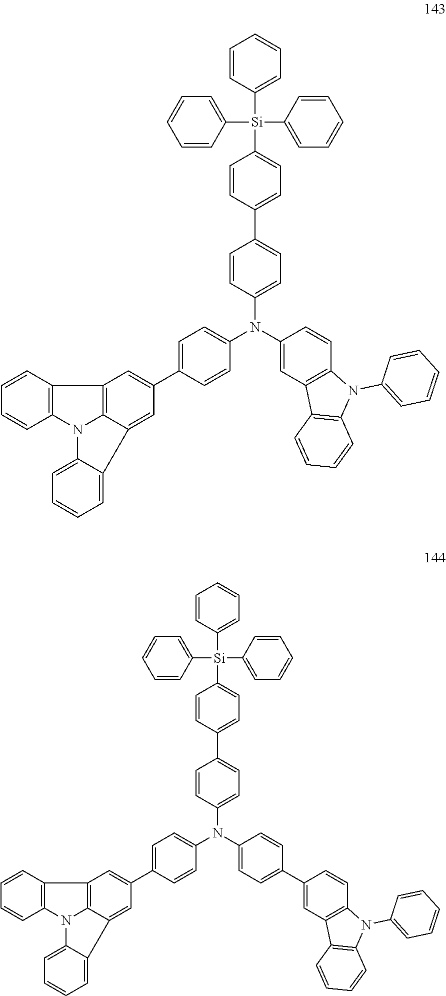 [SMILES: c1ccc(-n2c3ccccc3c3cc(N(c4ccc(-c5ccc([Si](c6ccccc6)(c6ccccc6)c6ccccc6)cc5)cc4)c4ccc(-c5cc6c7ccccc7n7c8ccccc8c(c5)c67)cc4)ccc32)cc1 ; c1ccc(-n2c3ccccc3c3cc(-c4ccc(N(c5ccc(-c6ccc([Si](c7ccccc7)(c7ccccc7)c7ccccc7)cc6)cc5)c5ccc(-c6cc7c8ccccc8n8c9ccccc9c(c6)c78)cc5)cc4)ccc32)cc1]

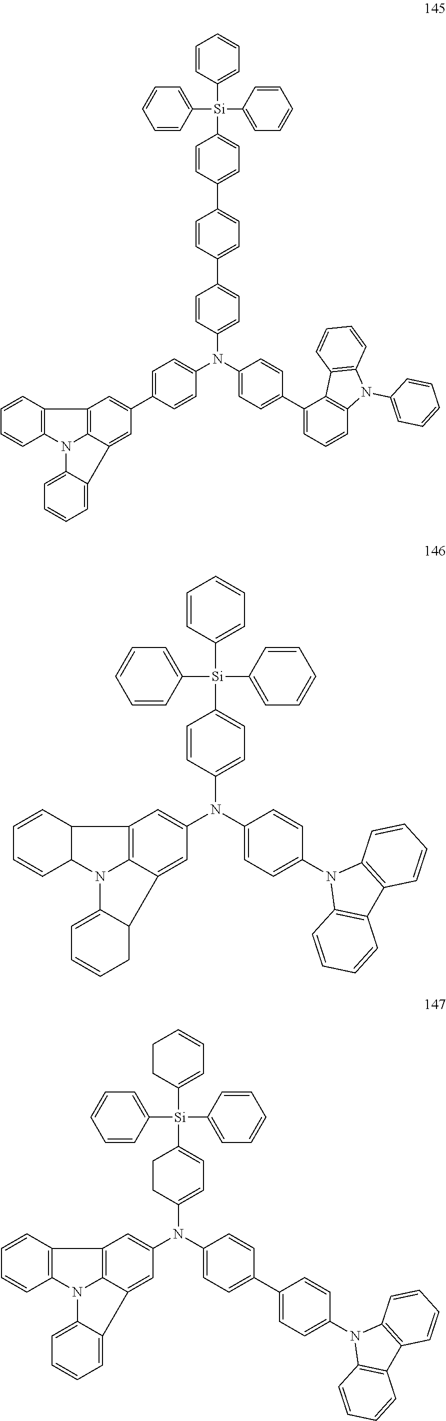 [SMILES: c1ccc(-n2c3ccccc3c3c(-c4ccc(N(c5ccc(-c6ccc(-c7ccc([Si](c8ccccc8)(c8ccccc8)c8ccccc8)cc7)cc6)cc5)c5ccc(-c6cc7c8ccccc8n8c9ccccc9c(c6)c78)cc5)cc4)cccc32)cc1 ; C1=CCC2C(=C1)N1c3c2cc(N(c2ccc(-n4c5ccccc5c5ccccc54)cc2)c2ccc([Si](c4ccccc4)(c4ccccc4)c4ccccc4)cc2)cc3C2C=CC=CC21 ; C1=CCCC([Si](C2=CC=C(N(c3ccc(-c4ccc(-n5c6ccccc6c6ccccc65)cc4)cc3)c3cc4c5ccccc5n5c6ccccc6c(c3)c45)CC2)(c2ccccc2)c2ccccc2)=C1]

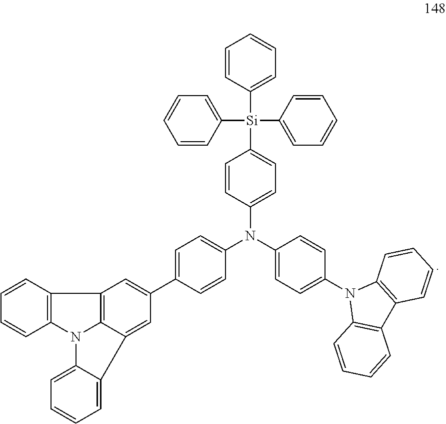 [SMILES: c1ccc([Si](c2ccccc2)(c2ccccc2)c2ccc(N(c3ccc(-c4cc5c6ccccc6n6c7ccccc7c(c4)c56)cc3)c3ccc(-n4c5ccccc5c5ccccc54)cc3)cc2)cc1]

16. The organic EL device as claimed in claim 4, wherein the material is one of the following Compounds 149 to 160:

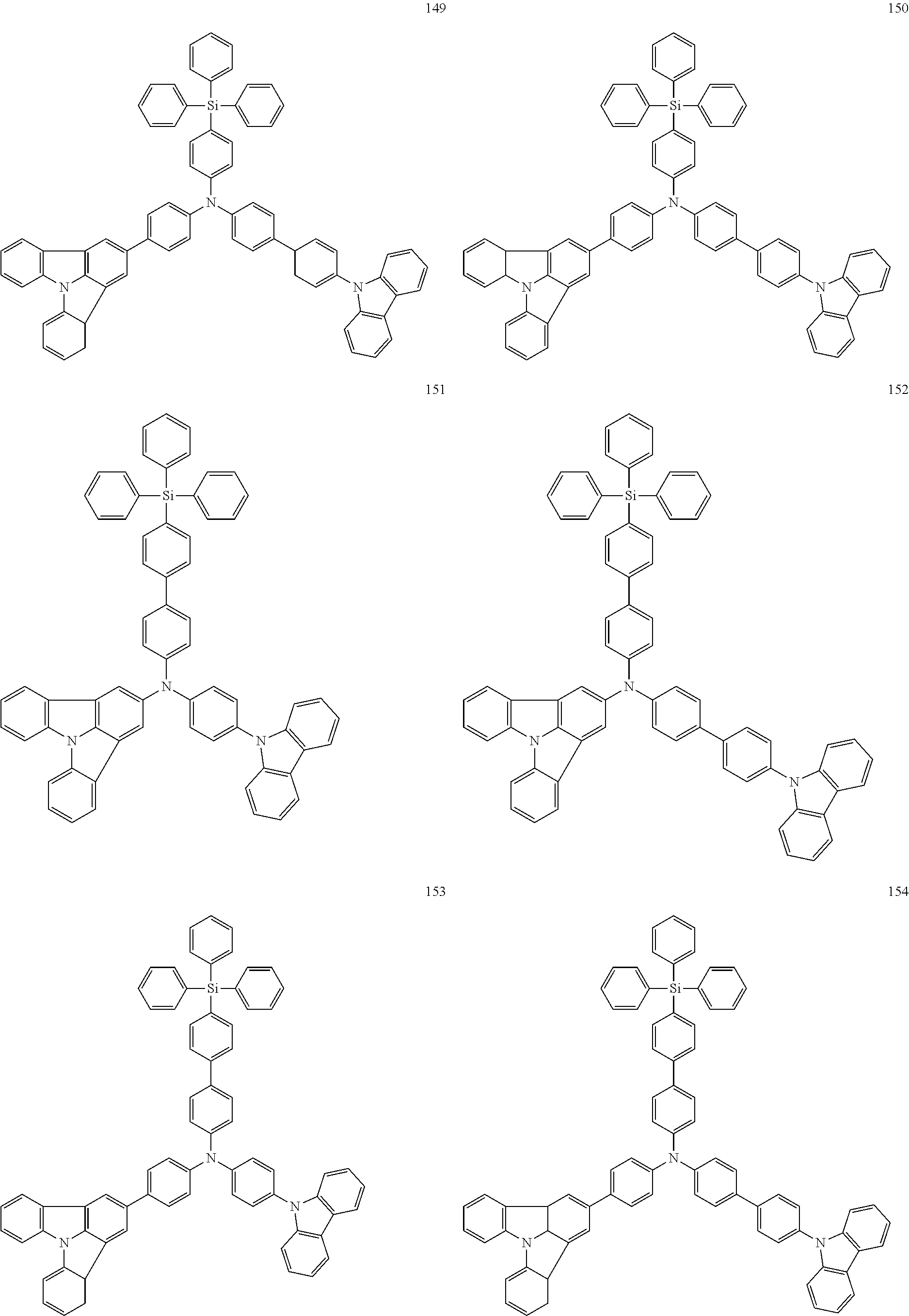

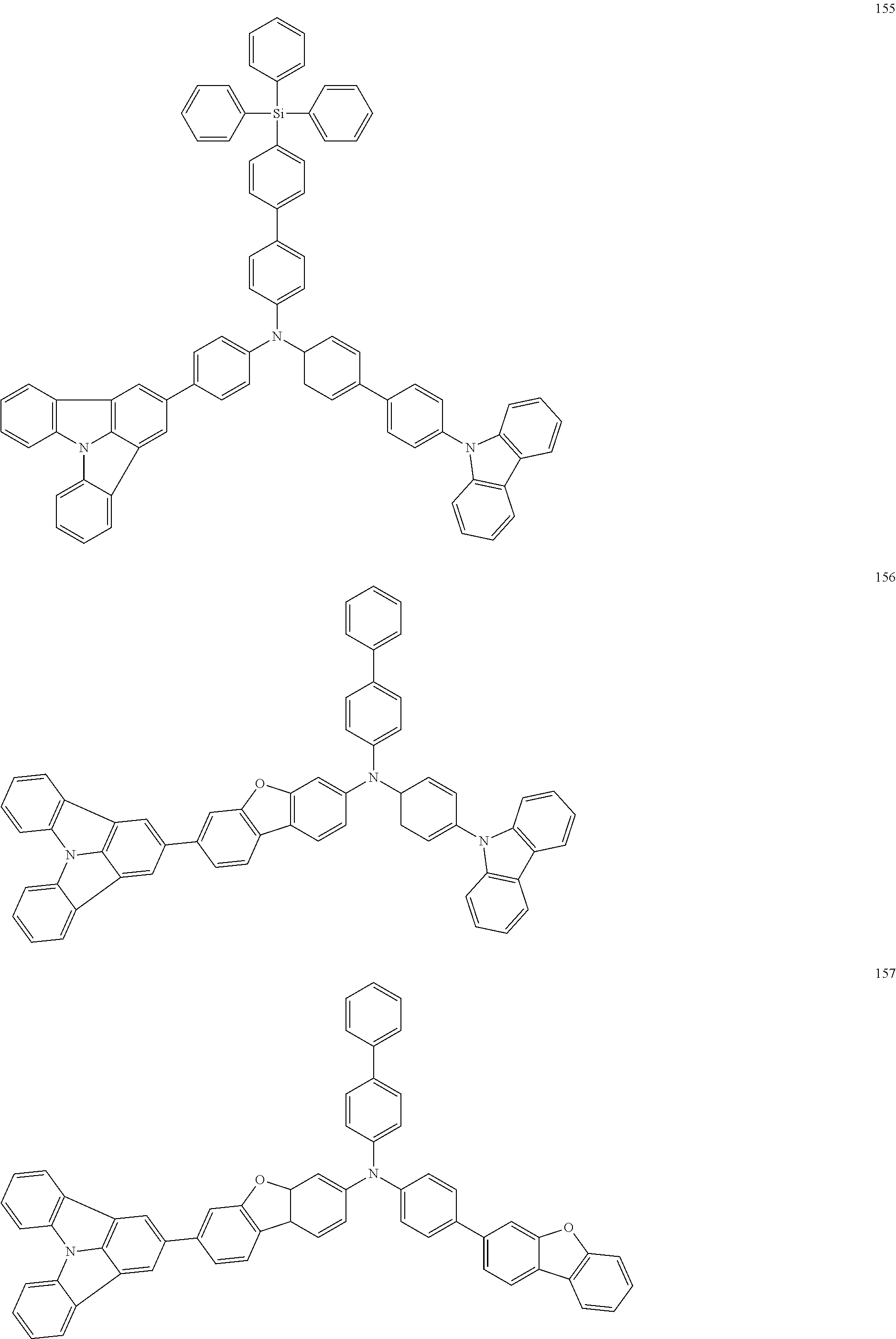 [SMILES: C1=CC(N(c2ccc(-c3ccc([Si](c4ccccc4)(c4ccccc4)c4ccccc4)cc3)cc2)c2ccc(-c3cc4c5ccccc5n5c6ccccc6c(c3)c45)cc2)CC=C1c1ccc(-n2c3ccccc3c3ccccc32)cc1 ; C1=CC(N(c2ccc(-c3ccccc3)cc2)c2ccc3c(c2)oc2cc(-c4cc5c6ccccc6n6c7ccccc7c(c4)c56)ccc23)CC=C1n1c2ccccc2c2ccccc21 ; C1=CC2c3ccc(-c4cc5c6ccccc6n6c7ccccc7c(c4)c56)cc3OC2C=C1N(c1ccc(-c2ccccc2)cc1)c1ccc(-c2ccc3c(c2)oc2ccccc23)cc1]

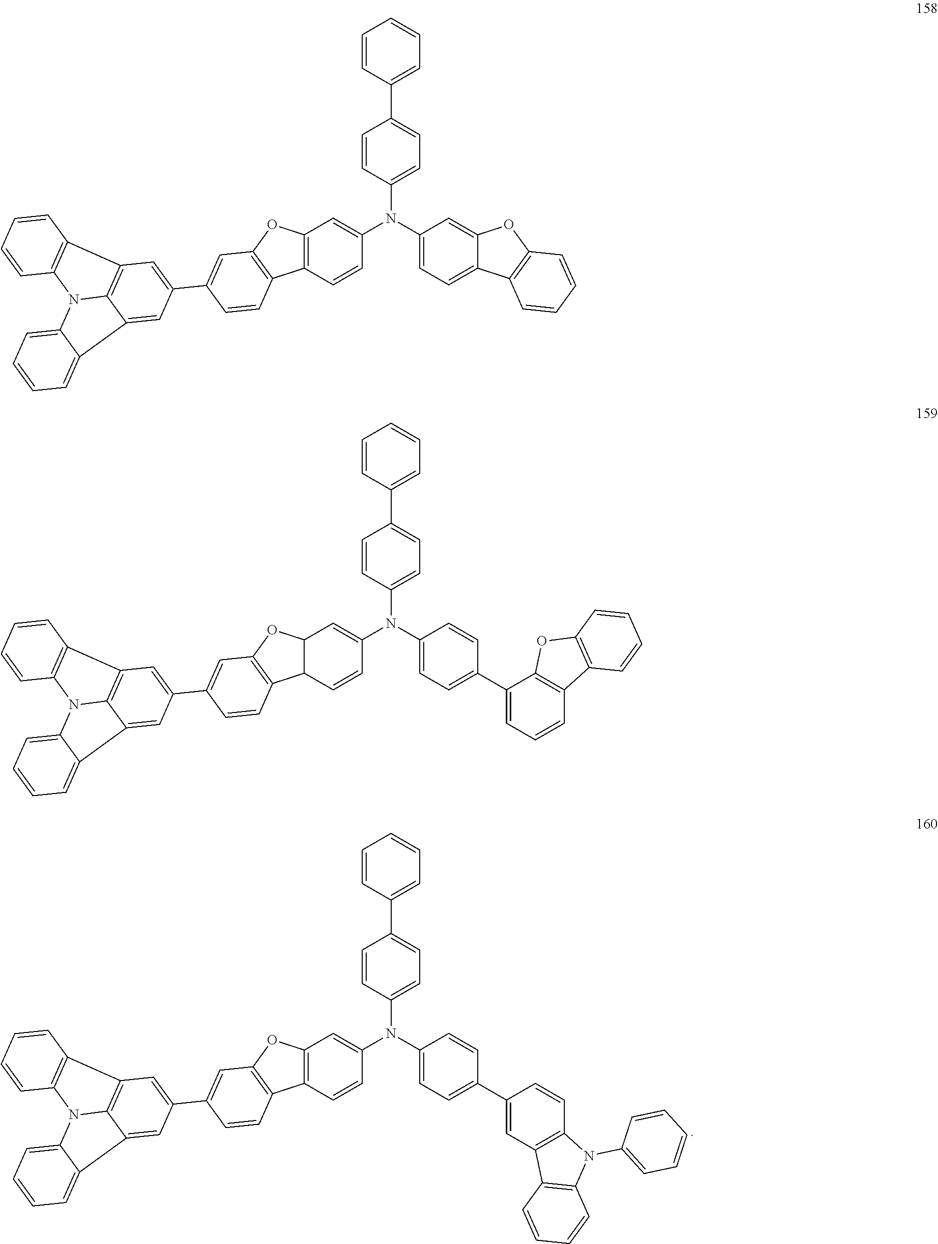 [SMILES: c1ccc(-c2ccc(N(c3ccc4c(c3)oc3ccccc34)c3ccc4c(c3)oc3cc(-c5cc6c7ccccc7n7c8ccccc8c(c5)c67)ccc34)cc2)cc1 ; C1=CC2c3ccc(-c4cc5c6ccccc6n6c7ccccc7c(c4)c56)cc3OC2C=C1N(c1ccc(-c2ccccc2)cc1)c1ccc(-c2cccc3c2oc2ccccc23)cc1 ; c1ccc(-c2ccc(N(c3ccc(-c4ccc5c(c4)c4ccccc4n5-c4ccccc4)cc3)c3ccc4c(c3)oc3cc(-c5cc6c7ccccc7n7c8ccccc8c(c5)c67)ccc34)cc2)cc1]

17. The organic EL device as claimed in claim 4, wherein the material is one of the following Compounds 161 to 170:

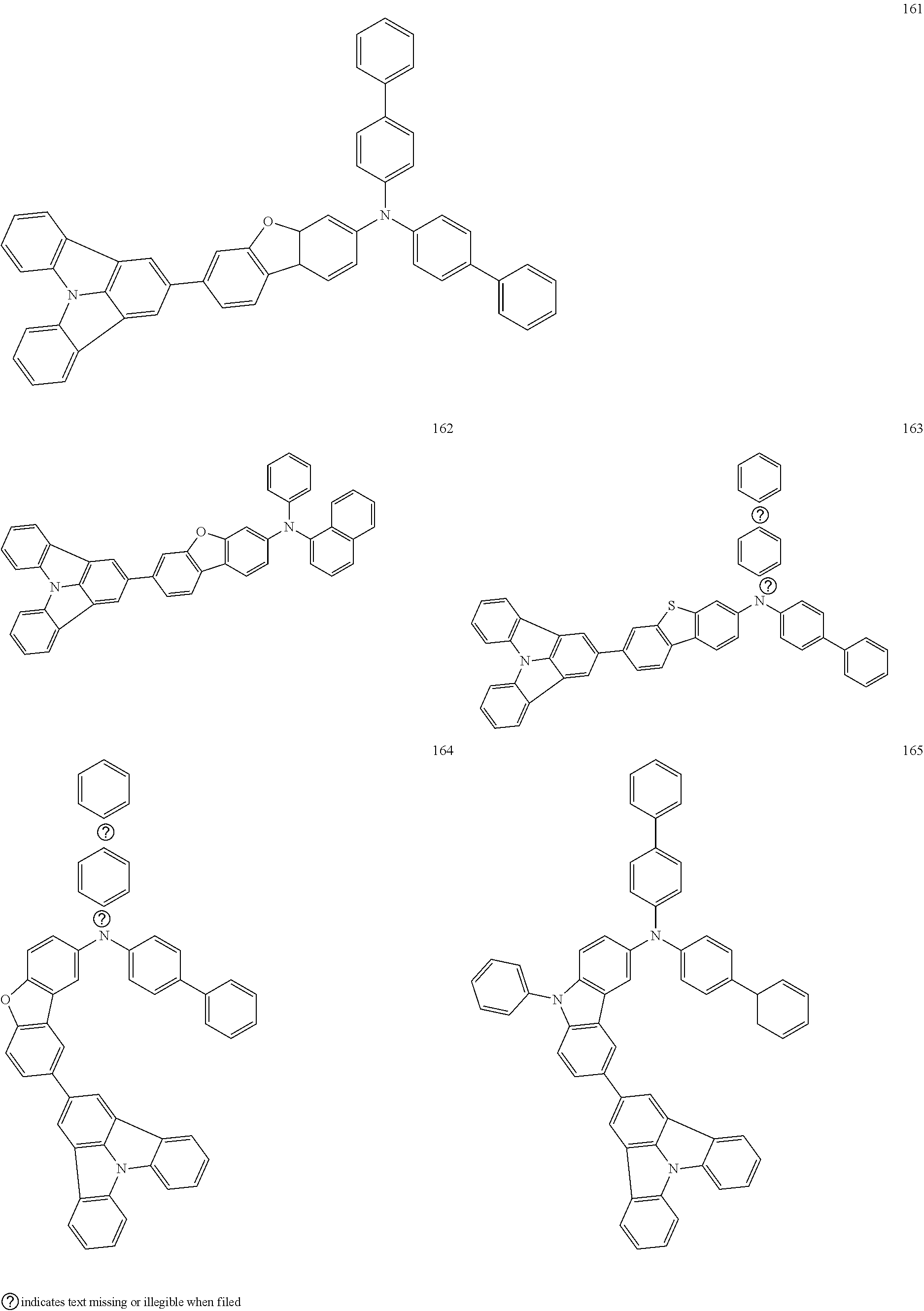

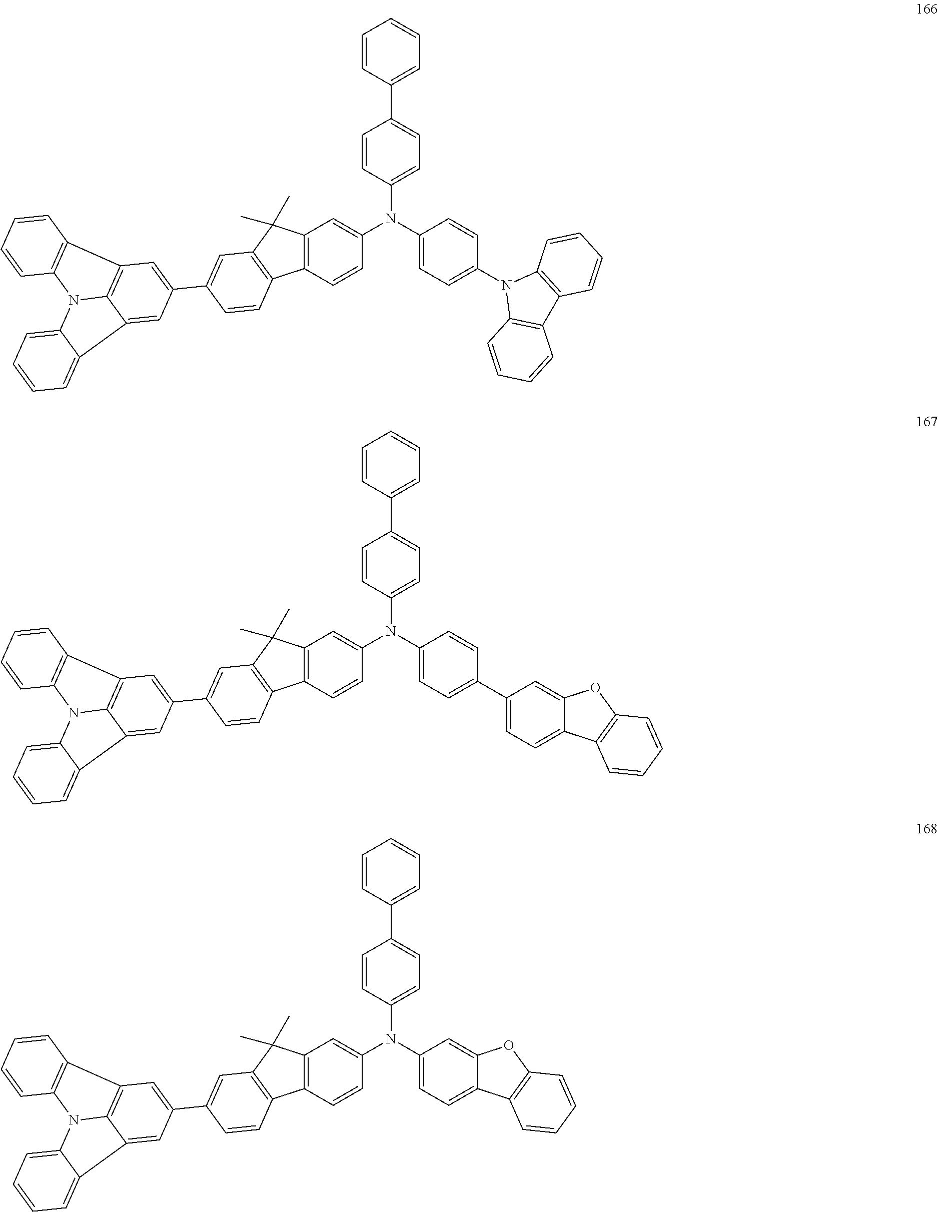 [SMILES: CC1(C)c2cc(-c3cc4c5ccccc5n5c6ccccc6c(c3)c45)ccc2-c2ccc(N(c3ccc(-c4ccccc4)cc3)c3ccc(-n4c5ccccc5c5ccccc54)cc3)cc21 ; CC1(C)c2cc(-c3cc4c5ccccc5n5c6ccccc6c(c3)c45)ccc2-c2ccc(N(c3ccc(-c4ccccc4)cc3)c3ccc(-c4ccc5c(c4)oc4ccccc45)cc3)cc21 ; CC1(C)c2cc(-c3cc4c5ccccc5n5c6ccccc6c(c3)c45)ccc2-c2ccc(N(c3ccc(-c4ccccc4)cc3)c3ccc4c(c3)oc3ccccc34)cc21]

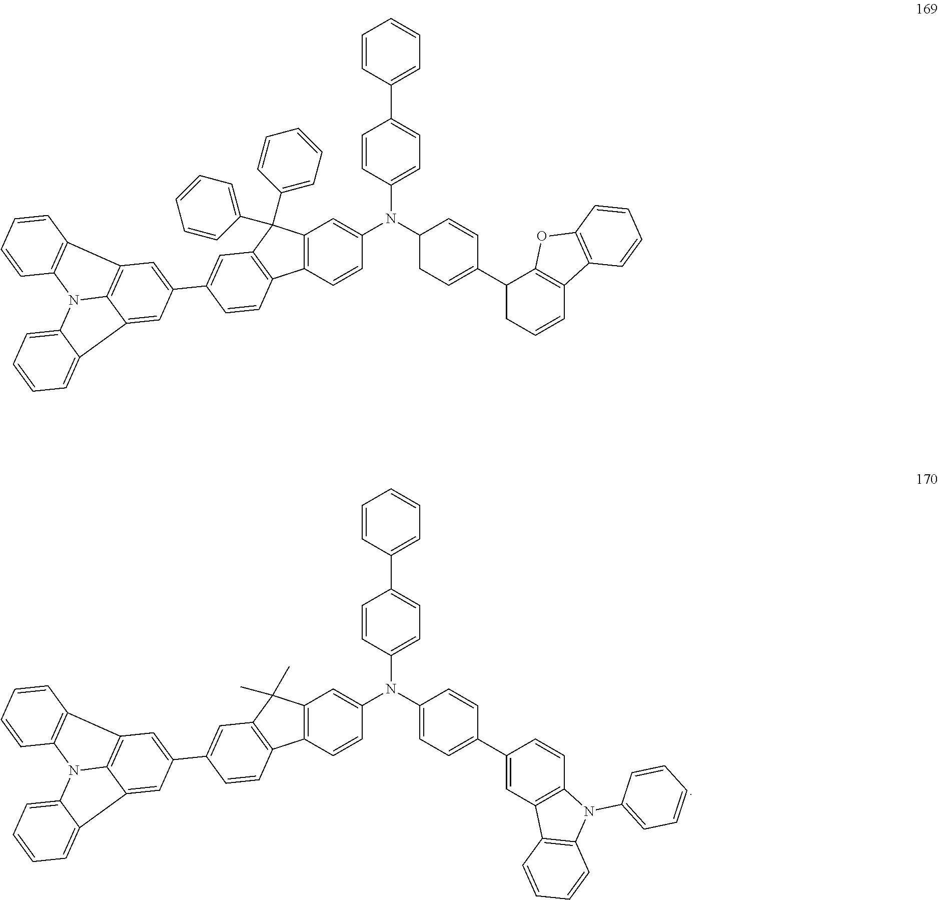 [SMILES: C1=Cc2c(oc3ccccc23)C(C2=CCC(N(c3ccc(-c4ccccc4)cc3)c3ccc4c(c3)C(c3ccccc3)(c3ccccc3)c3cc(-c5cc6c7ccccc7n7c8ccccc8c(c5)c67)ccc3-4)C=C2)C1 ; CC1(C)c2cc(-c3cc4c5ccccc5n5c6ccccc6c(c3)c45)ccc2-c2ccc(N(c3ccc(-c4ccccc4)cc3)c3ccc(-c4ccc5c(c4)c4ccccc4n5-c4ccccc4)cc3)cc21]

18. The organic EL device as claimed in claim 4, wherein:
at least one of Ar¹ and Ar² is a substituted or unsubstituted heteroaryl group, and
L¹, L², and L³ are each independently a single bond or a substituted or unsubstituted arylene group having 6 to 18 ring carbon atoms.

19. The organic EL device as claimed in claim 4, wherein Ar¹ and Ar² are each independently a substituted or unsubstituted aryl group having 6 to 24 ring carbon atoms for forming a ring, a substituted or unsubstituted carbazolyl group, a substituted or unsubstituted dibenzofuryl group, or a substituted or unsubstituted dibenzothienyl group.
